(19) United States
(12) Patent Application Publication
(10) Pub. No.: US 2026/0240673 A1
(43) Pub. Date: Aug. 20, 2026

(54) Heel Protector with Releasable Foot Engaging Section and Corresponding Rehabilitation Systems

(71) Applicant: Medline Industries LP, Northfield, IL (US)

(72) Inventors: Reesha Talati, Chicago, IL (US); Drew Phalen, Buffalo Grove, IL (US); Staci Wood, Bartlett, IL (US); Zachary Alban, Northfield, IL (US); Paul Paczocha, Arlington Heights, IL (US)

(21) Appl. No.: 19/054,162

(22) Filed: Feb. 14, 2025

(51) Int. Cl. A61F 5/01

(52) U.S. Cl. CPC A61F 5/0127; A61F 2005/0172

(57) ABSTRACT

A heel protector includes a leg engaging section with a first side leg engaging section, a second side leg engaging section, and a third side leg engaging section coupled between the first and second side leg engaging sections. A foot engaging section is coupled to one of the first or second side leg engaging sections and is selectively couplable to the other, defining a leg insertion aperture when coupled. The heel protector can lie in a substantially planar configuration when the foot engaging section is decoupled. The third side leg engaging section defines a heel receiver with a peninsular aperture. A side leg engaging section strap extends distally from the foot engaging section and is selectively couplable to one of the first or second side leg engaging sections. The leg engaging section comprises a compressible cushion layer made of organic or inorganic batting, or combinations thereof.

### BACKGROUND

### Technical Field

This disclosure relates generally to therapy systems, and more particularly to therapy systems for patient limbs below the knee.

### Background Art

Heel protectors are commonly used in medical rehabilitation applications to provide support and protection to the heel and surrounding areas. However, treating the sole of the foot can often be challenging, as existing heel protectors must be entirely removed for proper treatment. Therefore, there is a need for an improved heel protector that ensures optimal comfort and therapeutic benefits.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to further illustrate various embodiments and to explain various principles and advantages all in accordance with the present disclosure.

FIG. 1 illustrates a front elevation view of one explanatory heel protector configured in accordance with one or more embodiments of the disclosure.

FIG. 2 illustrates a rear elevation view of one explanatory heel protector configured in accordance with one or more embodiments of the disclosure.

FIG. 3 illustrates a patient’s limb being placed into a leg insertion aperture defined along a leg engaging section and a foot engaging section of one explanatory heel protector configured in accordance with one or more embodiments of the disclosure.

FIG. 4 illustrates one explanatory heel protector configured in accordance with one or more embodiments of the disclosure upon being applied to a patient’s limb.

FIG. 5 illustrates an alternate configuration of attachment straps of one explanatory heel protector configured in accordance with one or more embodiments of the disclosure.

FIG. 6 illustrates a patient’s limb being placed into a leg insertion aperture defined along a leg engaging section and a foot engaging section of an alternate configuration of one explanatory heel protector configured in accordance with one or more embodiments of the disclosure.

FIG. 7 illustrates a perspective view of one explanatory heel protector while in a substantially planar configuration with an attachment mechanism of the foot engaging section shown in a detached position from to the side leg engaging section in accordance with one or more embodiments of the disclosure.

FIG. 8 illustrates another perspective view of one explanatory heel protector while in a substantially planar configuration with an attachment mechanism of the foot engaging section shown in a detached position from to the side leg engaging section in accordance with one or more embodiments of the disclosure.

FIG. 9 illustrates a top plan view of one explanatory heel protector while in a substantially planar configuration with an attachment mechanism of the foot engaging section shown in a detached position from to the side leg engaging section in accordance with one or more embodiments of the disclosure.

FIG. 10 illustrates a bottom plan view of the heel protector while in a substantially planar configuration with an attachment mechanism of the foot engaging section shown in a detached position from to the side leg engaging section in accordance with one or more embodiments of the disclosure.

FIG. 11 illustrates a right elevation view of the heel protector while in a substantially planar configuration with an attachment mechanism of the foot engaging section shown in a detached position from to the side leg engaging section in accordance with one or more embodiments of the disclosure.

FIG. 12 illustrates a left elevation view of the heel protector while in a substantially planar configuration with an attachment mechanism of the foot engaging section shown in a detached position from to the side leg engaging section in accordance with one or more embodiments of the disclosure.

FIG. 13 illustrates a front elevation view of the heel protector while in a substantially planar configuration with an attachment mechanism of the foot engaging section shown in a detached position from to the side leg engaging section in accordance with one or more embodiments of the disclosure.

FIG. 14 illustrates a rear elevation view of the heel protector while in a substantially planar configuration with an attachment mechanism of the foot engaging section shown in a detached position from to the side leg engaging section in accordance with one or more embodiments of the disclosure.

FIG. 15 illustrates a first perspective view of one explanatory heel protector while in a wearable configuration with an attachment mechanism of the foot engaging section shown attached to the side leg engaging section in accordance with one or more embodiments of the disclosure.

FIG. 16 illustrates a second perspective view of one explanatory heel protector while in a wearable configuration with an attachment mechanism of the foot engaging section shown attached to the side leg engaging section in accordance with one or more embodiments of the disclosure.

FIG. 17 illustrates a top plan view of one explanatory heel protector while in a wearable configuration with an attachment mechanism of the foot engaging section shown attached to the side leg engaging section in accordance with one or more embodiments of the disclosure.

FIG. 18 illustrates a bottom plan view of one explanatory heel protector while in a wearable configuration with an attachment mechanism of the foot engaging section shown attached to the side leg engaging section in accordance with one or more embodiments of the disclosure.

FIG. 19 illustrates a right elevation view of one explanatory heel protector while in a wearable configuration with an attachment mechanism of the foot engaging section shown attached to the side leg engaging section in accordance with one or more embodiments of the disclosure.

FIG. 20 illustrates a left elevation view of one explanatory heel protector while in a wearable configuration with an attachment mechanism of the foot engaging section shown attached to the side leg engaging section in accordance with one or more embodiments of the disclosure.

FIG. 21 illustrates a front elevation view of one explanatory heel protector while in a wearable configuration with an attachment mechanism of the foot engaging section shown attached to the side leg engaging section in accordance with one or more embodiments of the disclosure.

FIG. 22 illustrates a front elevation view of one explanatory heel protector while in a wearable configuration with an attachment mechanism of the foot engaging section shown attached to the side leg engaging section in accordance with one or more embodiments of the disclosure.

FIG. 23 illustrates one or more embodiments of the disclosure.

FIG. 24 illustrates a front elevation view of another heel protector while in a substantially planar configuration with an attachment mechanism of the foot engaging section shown in a detached position from to the side leg engaging section in accordance with one or more embodiments of the disclosure.

FIG. 25 illustrates a rear elevation view of the heel protector of FIG. 24 while in a substantially planar configuration with an attachment mechanism of the foot engaging section shown in a detached position from to the side leg engaging section in accordance with one or more embodiments of the disclosure.

FIG. 26 illustrates a front elevation view of another heel protector while in a substantially planar configuration with an attachment mechanism of the foot engaging section shown in a detached position from to the side leg engaging section in accordance with one or more embodiments of the disclosure.

FIG. 27 illustrates a rear elevation view of the heel protector of FIG. 26 while in a substantially planar configuration with an attachment mechanism of the foot engaging section shown in a detached position from to the side leg engaging section in accordance with one or more embodiments of the disclosure.

FIG. 28 illustrates a front elevation view of another heel protector while in a substantially planar configuration with an attachment mechanism of the foot engaging section shown in a detached position from to the side leg engaging section in accordance with one or more embodiments of the disclosure.

FIG. 29 illustrates a rear elevation view of the heel protector of FIG. 28 while in a substantially planar configuration with an attachment mechanism of the foot engaging section shown in a detached position from to the side leg engaging section in accordance with one or more embodiments of the disclosure.

FIG. 30 illustrates a front elevation view of another heel protector while in a substantially planar configuration with an attachment mechanism of the foot engaging section shown in a detached position from to the side leg engaging section in accordance with one or more embodiments of the disclosure.

FIG. 31 illustrates a rear elevation view of the heel protector of FIG. 30 while in a substantially planar configuration with an attachment mechanism of the foot engaging section shown in a detached position from to the side leg engaging section in accordance with one or more embodiments of the disclosure.

FIG. 32 illustrates a front elevation view of another heel protector while in a substantially planar configuration with an attachment mechanism of the foot engaging section shown in a detached position from to the side leg engaging section in accordance with one or more embodiments of the disclosure.

FIG. 33 illustrates a rear elevation view of the heel protector of FIG. 32 while in a substantially planar configuration with an attachment mechanism of the foot engaging section shown in a detached position from to the side leg engaging section in accordance with one or more embodiments of the disclosure.

FIG. 34 illustrates a front elevation view of another heel protector while in a substantially planar configuration with an attachment mechanism of the foot engaging section shown in a detached position from to the side leg engaging section in accordance with one or more embodiments of the disclosure.

FIG. 35 illustrates a rear elevation view of the heel protector of FIG. 34 while in a substantially planar configuration with an attachment mechanism of the foot engaging section shown in a detached position from to the side leg engaging section in accordance with one or more embodiments of the disclosure.

FIG. 36 illustrates a front elevation view of another heel protector while in a substantially planar configuration with an attachment mechanism of the foot engaging section shown in a detached position from to the side leg engaging section in accordance with one or more embodiments of the disclosure.

FIG. 37 illustrates a rear elevation view of the heel protector of FIG. 36 while in a substantially planar configuration with an attachment mechanism of the foot engaging section shown in a detached position from to the side leg engaging section in accordance with one or more embodiments of the disclosure.

FIG. 38 illustrates a front elevation view of another heel protector while in a substantially planar configuration with an attachment mechanism of the foot engaging section shown in a detached position from to the side leg engaging section in accordance with one or more embodiments of the disclosure.

FIG. 39 illustrates a rear elevation view of the heel protector of FIG. 38 while in a substantially planar configuration with an attachment mechanism of the foot engaging section shown in a detached position from to the side leg engaging section in accordance with one or more embodiments of the disclosure.

FIG. 40 illustrates a front elevation view of another heel protector while in a substantially planar configuration with an attachment mechanism of the foot engaging section shown in a detached position from to the side leg engaging section in accordance with one or more embodiments of the disclosure.

FIG. 41 illustrates a rear elevation view of the heel protector of FIG. 40 while in a substantially planar configuration with an attachment mechanism of the foot engaging section shown in a detached position from to the side leg engaging section in accordance with one or more embodiments of the disclosure.

FIG. 42 illustrates a front elevation view of another heel protector while in a substantially planar configuration with an attachment mechanism of the foot engaging section shown in a detached position from to the side leg engaging section in accordance with one or more embodiments of the disclosure.

FIG. 43 illustrates a rear elevation view of the heel protector of FIG. 42 while in a substantially planar configuration with an attachment mechanism of the foot engaging section shown in a detached position from to the side leg engaging section in accordance with one or more embodiments of the disclosure.

FIG. 44 illustrates a front elevation view of another heel protector while in a substantially planar configuration with an attachment mechanism of the foot engaging section shown in a detached position from to the side leg engaging section in accordance with one or more embodiments of the disclosure.

FIG. 45 illustrates a rear elevation view of the heel protector of FIG. 44 while in a substantially planar configuration with an attachment mechanism of the foot engaging section shown in a detached position from to the side leg engaging section in accordance with one or more embodiments of the disclosure.

FIG. 46 illustrates a front elevation view of another heel protector while in a substantially planar configuration with an attachment mechanism of the foot engaging section shown in a detached position from to the side leg engaging section in accordance with one or more embodiments of the disclosure.

FIG. 47 illustrates a rear elevation view of the heel protector of FIG. 46 while in a substantially planar configuration with an attachment mechanism of the foot engaging section shown in a detached position from to the side leg engaging section in accordance with one or more embodiments of the disclosure.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present disclosure.

### DETAILED DESCRIPTION OF THE DRAWINGS

Embodiments of the disclosure are now described in detail. Referring to the drawings, like numbers indicate like parts throughout the views. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.” Relational terms such as first and second, top and bottom, and the like may be used solely to distinguish one entity or action from another entity or action without necessarily requiring or implying any actual such relationship or order between such entities or actions.

The terms “substantially,” “essentially,” “approximately,” “about,” or any other version thereof, are defined as being close to as understood by one of ordinary skill in the art, and in one non-limiting embodiment the term is defined to be within ten percent, in another embodiment within five percent, in another embodiment within one percent and in another embodiment within one-half percent. Also, reference designators shown herein in parenthesis indicate components shown in a figure other than the one in discussion. For example, talking about a device (10) while discussing figure A would refer to an element, 10, shown in figure other than figure A.

Embodiments of the disclosure provide heel protectors that are designed to provide support and protection to the heel and surrounding areas. In one or more embodiments, a heel protector configured in accordance with one or more embodiments of the disclosure comprises three leg engaging sides, namely, a first side leg engaging section, a second side leg engaging section, and a third side leg engaging section. In one or more embodiments, a foot engaging section is fixedly coupled to one of the side leg engaging sections and is further selectively attachable to another of the side leg engaging sections.

In one or more embodiments, the side leg engaging sections can be folded to form a three-sided shape configured to surround three sides of a patient’s leg. When the foot engaging section is selectively coupled to the side leg engaging section to which it is not fixedly attached, the heel protector defines a leg insertion aperture into which a patient can insert a leg. When the foot engaging section is selectively attached to its corresponding side leg engaging section, the resulting heel protector provides a secure and comfortable fit around a patient’s leg. By contrast, when the foot engaging section is detached from that side leg engaging section, the various sections of the heel protector can unfold so as to lie in a substantially planar configuration.

In one or more embodiments, the heel protector also comprises side leg engaging section straps that extend distally from the foot engaging section. In one or more embodiments, these side leg engaging section straps can attach to fastener panels, one example of which includes a loop fastener panel, which is situated on an exterior surface of the side leg engaging sections. In so doing, the side leg engaging section straps provide additional support and stability.

In one or more embodiments, the heel protector also comprises a foot engaging section having shin straps straps that extend distally from the side leg engaging section. In one or more embodiments, these shin straps can attach to fastener panels, one example of which includes a loop fastener panel, which is situated on an exterior surface of the side leg engaging sections. In so doing, the shin straps provide additional support and stability.

In one or more embodiments, hook and loop fasteners can be used for attachment purposes. Illustrating by example, in one or more embodiments the foot engaging section can selectively attach to the side leg engaging section using hook and loop fasteners. Similarly, in one or more embodiments the side leg engaging straps can include hook fasteners that attach to loop fastener panels.

Thus, the foot engaging section and side leg engaging sections of the heel protector can utilize hook and loop fasteners for secure attachment. However, it should be noted that other types of fasteners can also be used in place of hook and loop fasteners, depending on the specific requirements and preferences. Some examples of substitutes for hook and loop fasteners include snap buttons, buckles, magnetic closures, adhesive strips, zipper closures, or even adjustable straps with clasps. These alternative fastening mechanisms offer flexibility in design and can be chosen based on factors such as ease of use, durability, adjustability, and aesthetic appeal. The embodiments of the disclosure provide the option to select the most suitable fastening method for achieving a secure and comfortable fit between the foot engaging section and the side leg engaging sections of the heel protector.

In one or more embodiments, the foot engaging sections are attached to the side leg engaging sections using tabs and hook fasteners. In one or more embodiments, the tabs extend from one side of the foot engaging sections and can be stitched to the side leg engaging sections. In one or more embodiments, the foot engaging sections and the tabs define rectangular apertures when attached, allowing for additional breathability into the foot receiving chamber of the heel protector.

Embodiments of the disclosure offer unique attachment mechanisms between the foot engaging sections and the side leg engaging sections. In one or more embodiments, the tabs, straps, and fasteners, including their placement, location, shapes, angles, and directions, ensure secure and comfortable fits for the wearers. Embodiments of the disclosure have potential applications in various fields, including foot rehabilitation, medical applications, sports rehabilitation equipment, and any wearables that require stable connections between the foot and leg sections of a heel protector. Compared to existing solutions, embodiments of the disclosure offer superior ergonomic benefits and more secure attachment mechanisms.

In one or more embodiments, a heel protector comprises a leg engaging section defined by three side leg engaging sections. A foot engaging section is fixedly attached to one of the side leg engaging sections and can be selectively attached to another of the side leg engaging sections. In one or more embodiments, when the foot engaging section is selectively attached to a side leg engaging section, the resulting structure defines a leg insertion aperture allowing a patient to insert their leg into the heel protector. By contrast, when the foot engaging section is detached from the side leg engaging section, the various sections of the heel protector can pivot relative to each other so as to lie in a substantially planar configuration.

In one or more embodiments, the heel protector may include a heel receiver defined by a peninsular aperture centrally situated at an edge of one of the side leg engaging sections. The heel protector may further comprise a side leg engaging section strap extending distally from the foot engaging section, which can be selectively attached to one of the side leg engaging sections. In one or more embodiments, the side leg engaging section strap extends at a different angle from the foot engaging section than do other tabs. Moreover, in one or more embodiments the side leg engaging section strap extends distally farther from the foot engaging section than do the tabs. The leg engaging section and the side leg engaging sections may comprise a compressible cushion layer made of organic batting, inorganic batting, or combinations thereof.

In one or more embodiments, the heel protector may include loop fastener panels coupled to the exterior surfaces of the side leg engaging sections. As noted above, fasteners other than hook and loop fasteners can be used. Accordingly, the heel protector may include other types of fastener panels coupled to the exterior surfaces of the side leg engaging sections.

In one or more embodiments, the foot engaging section may also have at least one tab that is shorter than the side leg engaging section strap. In one or more embodiments, the at least one tab can be selectively attachable to one of the side leg engaging sections. The at least one tab may include a hook fastener for secure attachment. The tabs extend from the foot engaging section and may have rounded corners.

In one or more embodiments, the foot engaging section may be coupled to one of the side leg engaging sections by tabs, together defining an aperture between a side leg engaging sections. The heel protector may also include a compressible cushion layer made of organic batting, inorganic batting, or combinations thereof. The embodiments of the disclosure provide a unique attachment mechanism for a foot engaging section to side leg engaging sections, ensuring a secure and comfortable fit for the wearer. Other advantages will be described below, still others will be obvious to those of ordinary skill in the art having the benefit of this disclosure.

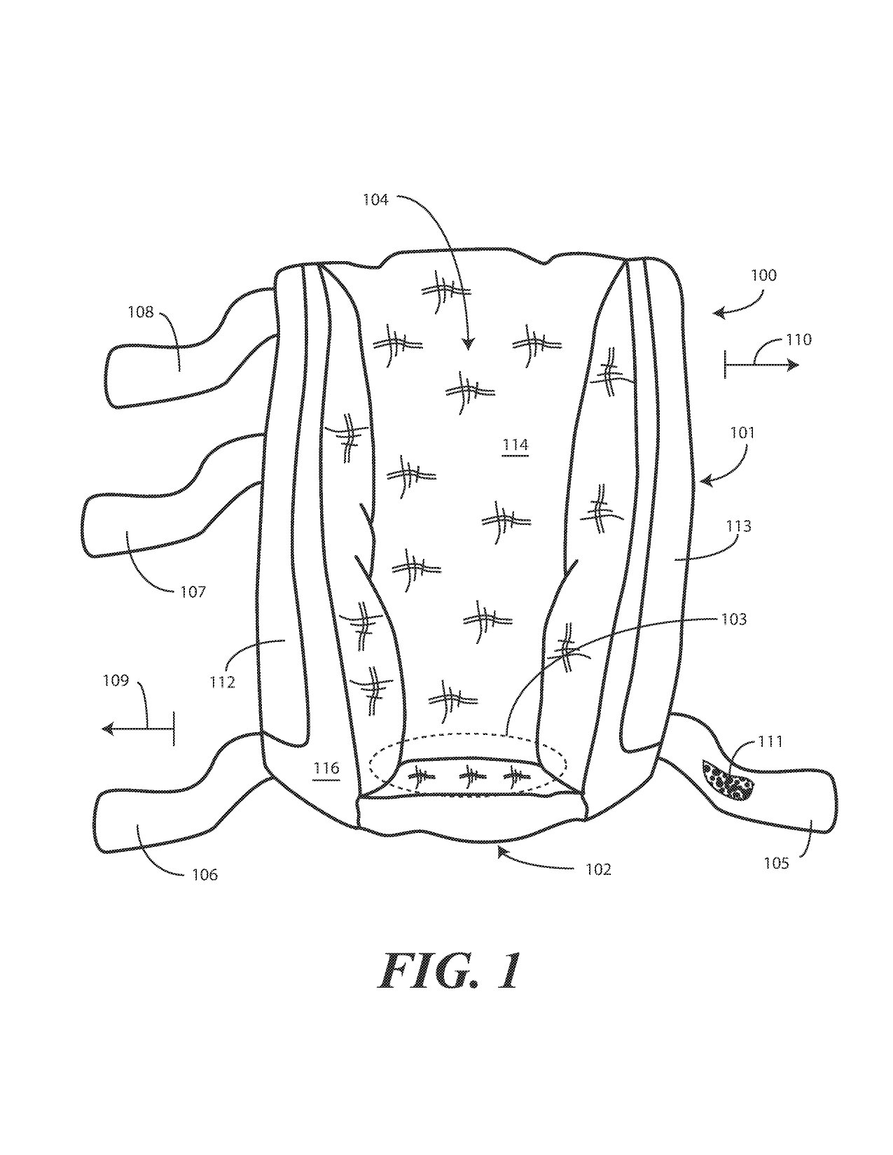

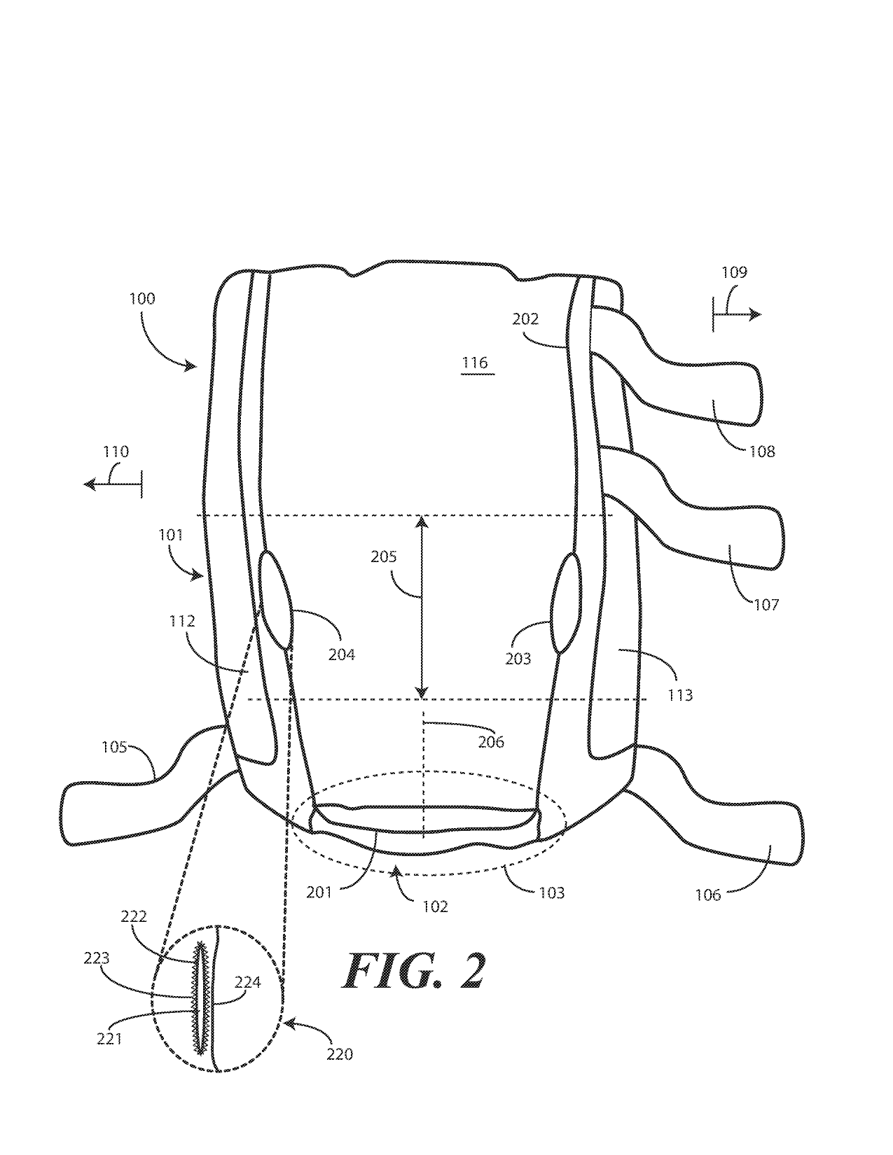

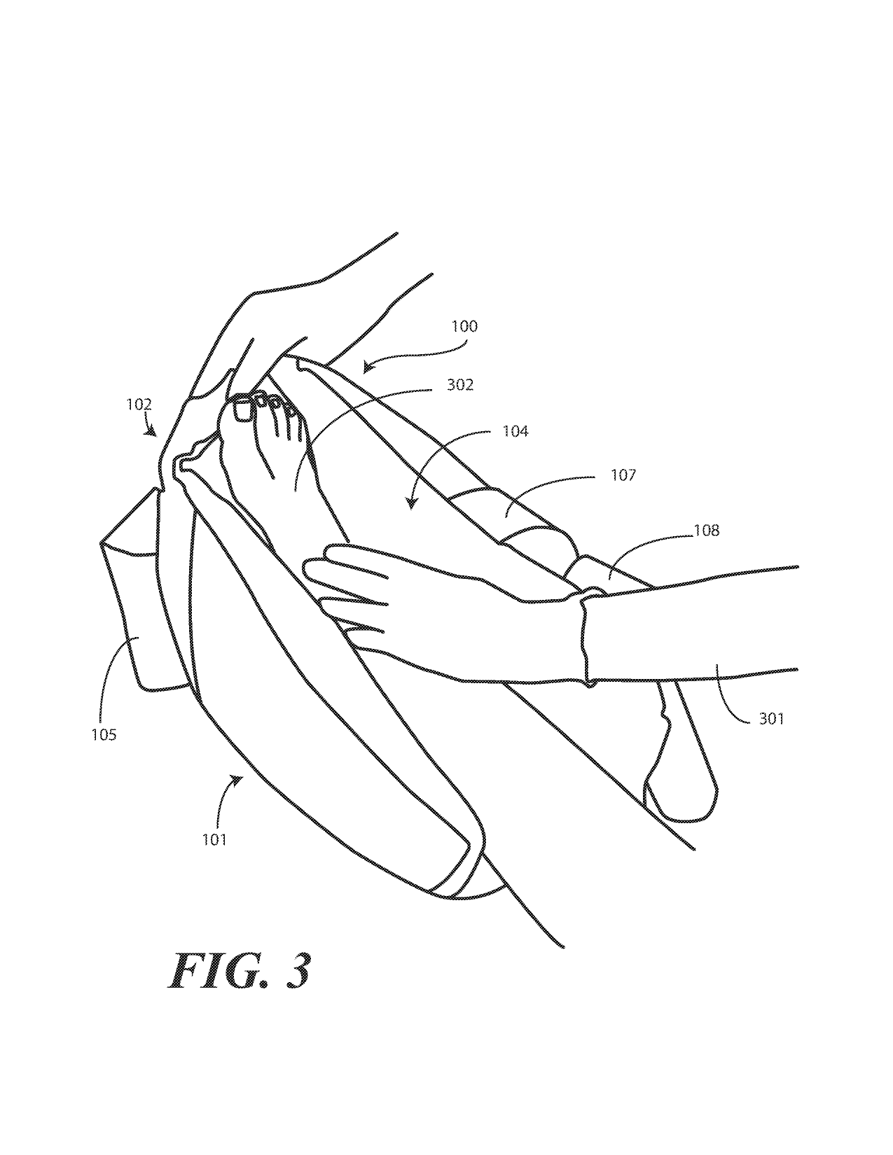

Turning now to FIGS. 1 and 2, illustrated therein is one explanatory embodiment of a heel protector 100 configured in accordance with one or more embodiments of the disclosure. In the illustrative embodiment of FIGS. 1 and 2, the heel protector 100 includes a leg engaging section 101 and a foot engaging section 102. The leg engaging section 101 intersects the foot engaging section 102 at a heel receiver 103. In one embodiment, the heel receiver 103 defines an aperture 201 through which a patient’s heel can be seen when the heel protector 100 is applied to the patient’s leg. The leg engaging section 101 and the foot engaging section 102 have defined there along a leg insertion aperture 104. A patient’s leg can be inserted through the leg insertion aperture 104, as will be shown in FIG. 3 below. In one or more embodiments, the heel receiver 103 is configured to remove pressure from the heel of a patient when the heel protector 100 is being worn.

In one embodiment, the heel protector 100 includes one or more fastening straps 105,106,107,108 extending from the sides of the leg engaging section 101, the foot engaging section, or combinations thereof. For example, in the illustrative embodiment of FIGS. 1 and 2, the heel protector 100 has four fastening straps 105,106,107,108 extending from its sides. At least one fastening strap 105 extends from a first side of the heel protector 100, while others extend from another side of the heel protector 100. This allows the fastening straps to “crisscross” from one side of the heel protector 100 to the other.

In this illustrative embodiment, two fastening straps 105,106 extend from the foot engaging section 102, while two other fastening straps 107,108 extend from the leg engaging section 101. Also, in this illustrative embodiment, three fastening straps 106,107,108 extend from the medial side 109 of the heel protector 100, while one fastening strap 105 extends from the lateral side 110 of the heel protector 100. This configuration is illustrative only, as other configurations and placements of the fastening straps 105,106,107,108 will be obvious to those of ordinary skill in the art having the benefit of this disclosure. Some examples of this will be illustrated and described with reference to subsequent figures.

In one embodiment, the fastening straps 105,106,107,108 are stretchable. For example, they may comprise an elasticized material configured to stretch when being wrapped about the leg insertion aperture 104. In another embodiment, the fastening straps 105,106,107,108 are not stretchable, but are rather material layers that are fixed in length and do not change when being wrapped about the leg insertion aperture 104. The fastening straps 105,106,107,108 are affixed to the heel protector 100 by stitching in one embodiment. FIG. 2 illustrates fastening straps 107,108 being attached to the leg engaging section 101 along seam 202.

In one embodiment, each of the fastening straps 105,106,107,108 comprises one of a hook fastener or a loop fastener disposed there along. Illustrating by example, fastening strap 105 may have hook fasteners disposed along side 111. To complete the fastening system, in one embodiment the leg engaging section 101 includes one or more panels 112,113 that have a complementary fastener disposed there along.

Where, for example, fastening strap 105 includes hook fasteners, corresponding panel 112 may have loop fasteners disposed there along, as the loop fasteners are complementary to the hook fasteners. Accordingly, when fastening strap 105 is wrapped across the leg insertion aperture 104, it can be attached anywhere along panel 112. The same is true with fastening straps 106,107,108 attaching to panel 113.

While hook and loop fasteners are one type of fastener or attachment mechanism suitable for use with embodiments of the disclosure, it should be noted that others will be obvious to those having ordinary skill in the art and the benefit of this disclosure. For example, the hook and loop fasteners can be replaced by laces, snaps, buttons, drawstrings, or other fastening devices.

In one embodiment, the interior lining 114 of the heel protector is soft and comfortable. For example, in one embodiment the interior lining 114 can be fleece or another soft material. In another embodiment, the interior lining 114 can be felt or chamois. As noted above, in one embodiment the heel protector 100 is configured to be used with a compression device. Accordingly, in one embodiment the interior lining 114 can be configured to attach, mate, or otherwise interface with an exterior lining of the compression device.

In one embodiment, the interior lining 114 has a relatively high coefficient of friction so that the heel protector 100 does not move when wrapped about a patient’s limb or compression device attached thereto. For example, the interior lining 114 can be brushed, knapped, or sanded to raise its pile for comfort and increase the coefficient of friction.

In one embodiment, the interior lining 114 has an antibacterial, antimicrobial, or anti-odor material integrated therein to help reduce the risk of bacteria, microbes, or odors from existing in the interior of the heel protector 100 after prolonged use. The interior lining 114 can also be manufactured from a wicking material. In other embodiments, the interior lining 114 can also be manufactured from an absorbing material. The exterior 116 of the heel protector 100 may be water resistant or waterproof as desired. In one embodiment, the interior of the heel protector 100 can be constructed from a cooling material, such as a gel that can be cooled to apply thermal therapy to the patient.

As shown in FIG. 2, in one embodiment the leg engaging section 101 defines at least one aperture 203,204 disposed in an ankle region 205 of the leg engaging section 101. In one embodiment, the at least one aperture 203,204 is disposed at least a predetermined distance 206 from the foot engaging section 102.

For example, in one embodiment, aperture 203 is disposed approximately six inches proximally from the aperture 201 disposed in the heel receiver 103. In one or more embodiments, separating the at least one aperture 203,204 from the foot engaging section 102 by the predetermined distance 206 advantageously allows the heel protector 100 to be used with compression devices having connection tubes exiting therefrom at non-orthogonal angles. In one embodiment, the predetermined distance 206 would be about six inches.

In one embodiment, the aperture 203 is disposed about one inch superior to the posterior aspect (which runs along the line of predetermined distance 206) of the heel protector 100. Similarly, another aperture 204 can be disposed as a mirror image of the first aperture 203. In one or more embodiments, the heel protector 100 is configured for use as one component in a rehabilitation system, with a corresponding component comprising a compression device. Where the compression device includes a connection tube extending from an inflatable bladder, the inclusion of apertures 203,204 help to minimize the risk of the connection tube contacting a patient’s skin by providing an easy and convenient exit port. The addition of the apertures 203,204 on the lateral side 110 and medial side 109 of the leg engaging section 101 allows the connection tube emanating from the compression device to run directly out of the heel protector 100, thereby eliminating the need to “tuck” tubing into the seam of the boot and away from the skin. In one embodiment, one aperture 203 is disposed about forty-five degrees around the leg engaging section 101 from the other aperture 204.

As shown in the exploded view 220, in one embodiment the aperture 204 can be configured as a channel 221 to permit a connection tube extending from an inflatable bladder of a compression device to pass therethrough. The channel 221 can be reinforced about its perimeter 222. For instance, in one embodiment, the channel 221 is reinforced with stitching 223. The channel 221 can be disposed in-line with a seam 224 of the heel protector 100, or as shown in the exploded view 220, can be proximally located with the seam 224.

In one embodiment, the apertures 203,204 are color-coded. As will be described below, embodiments of compression devices configured in accordance with one or more embodiments of the disclosure can be color-coded in accordance with manufacturer, size, or other considerations. Accordingly, in one embodiment the apertures 203,204 can be correspondingly color-coded. Where the apertures 203,204 are configured to be used with differently color-coded compression devices, they may be color-coded differently. Where the apertures 203,204 are to be used with a common compression device, they may be similarly color-coded.

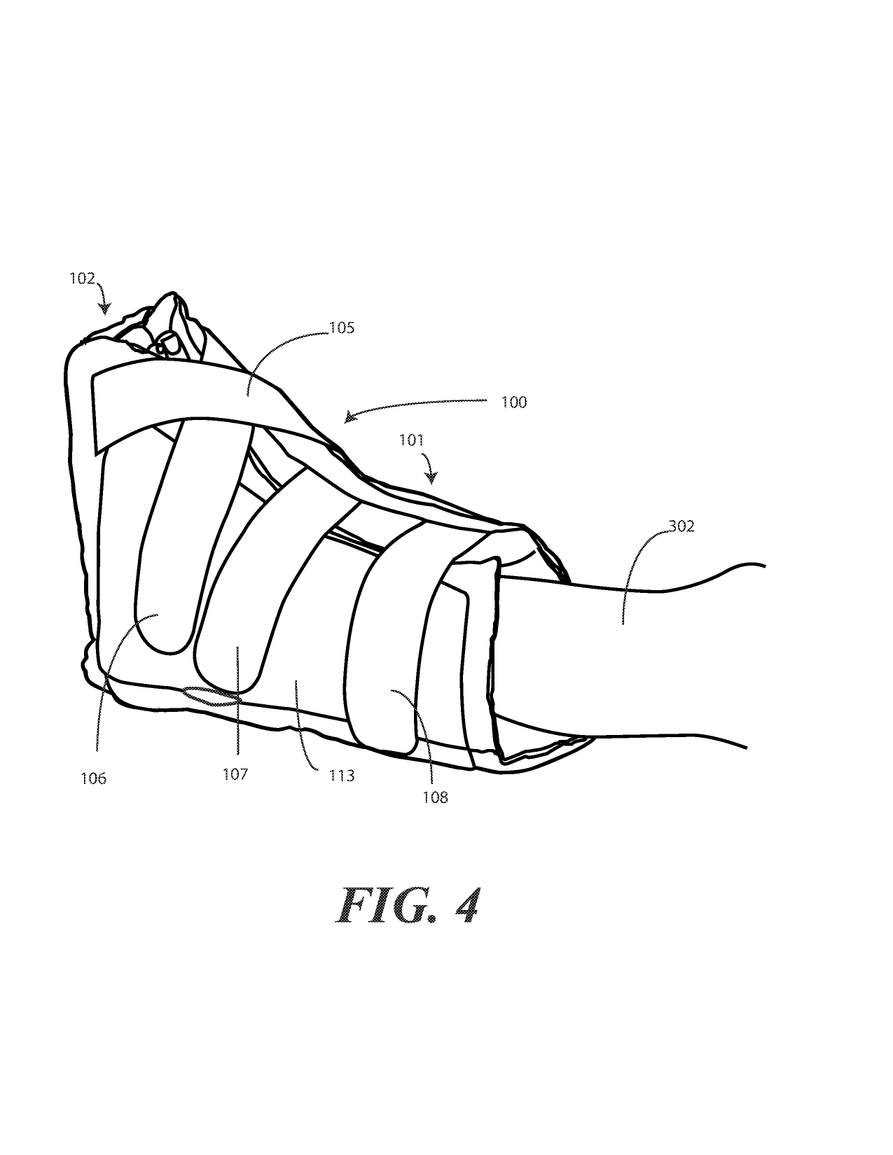

Turning now to FIGS. 3 and 4, a method of applying a heel protector 100 is illustrated. As shown in FIG. 3, a health care services provider 301 passes a patient’s leg 302 through the leg insertion aperture 104 disposed along the leg engaging section 101 and the foot engaging section 102 such that the patient’s heel engages the heel receiver (103). Once this step is complete, the health care services provider 301 will wrap the fastening straps 105,107,108 across the leg insertion aperture 104 to retain the heel protector 100 to the patient’s leg 302. The result of this wrapping is shown in FIG. 4.

As shown in FIG. 4, three of the fastening straps 106,107,108 have been wrapped about the leg insertion aperture 104 and attached to panel 113. Similarly, fastening strap 105 has been wrapped about the leg insertion aperture 104 and attached to panel (112). This retains the heel protector 100 to the patient’s leg 302.

As shown in FIG. 4, fastening strap 105 and fastening strap 106 have been “crisscrossed.” Fastening straps 107,108 could have been similarly crisscrossed but have been left in a substantially parallel configuration in this illustrative embodiment. In one embodiment, the health care services provider (301) is instructed to achieve this configuration as follows: after inserting the patient’s leg 302 into the leg insertion aperture 104, fastening strap 108 is to be wrapped about the leg insertion aperture 104 and attached to panel 113. Next, fastening strap 107 is to be wrapped about the leg insertion aperture 104 and attached to panel 113 to securely affix the leg engaging section 101 about the patient’s leg 302. While fastening straps 107,108 can be attached so that they are substantially parallel, in one embodiment the health care services provider (301) is instructed to cause fastening strap 107 to extend toward the foot engaging section 102, and thereby non-parallel relative to fastening strap 108, to achieve a more “snug” fit.

Next, the health care services provider (301) is instructed to wrap fastening strap 106 across the leg insertion aperture 104 in a substantially diagonal configuration to attach to panel 113. Corresponding fastening strap 105 can then crisscross over fastening strap 106 to attach to panel (112). Fastening straps 105 and 106 work to retain the foot engaging section 102 to the patient’s foot.

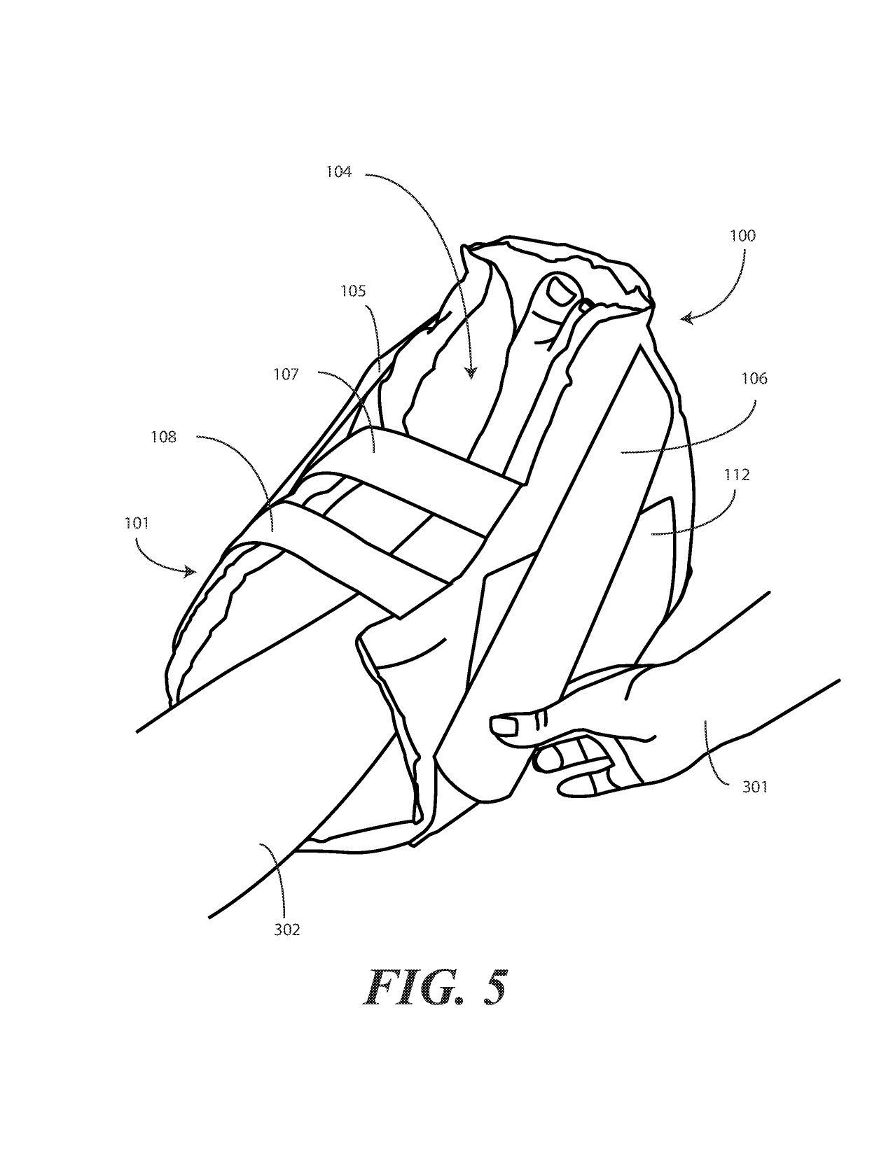

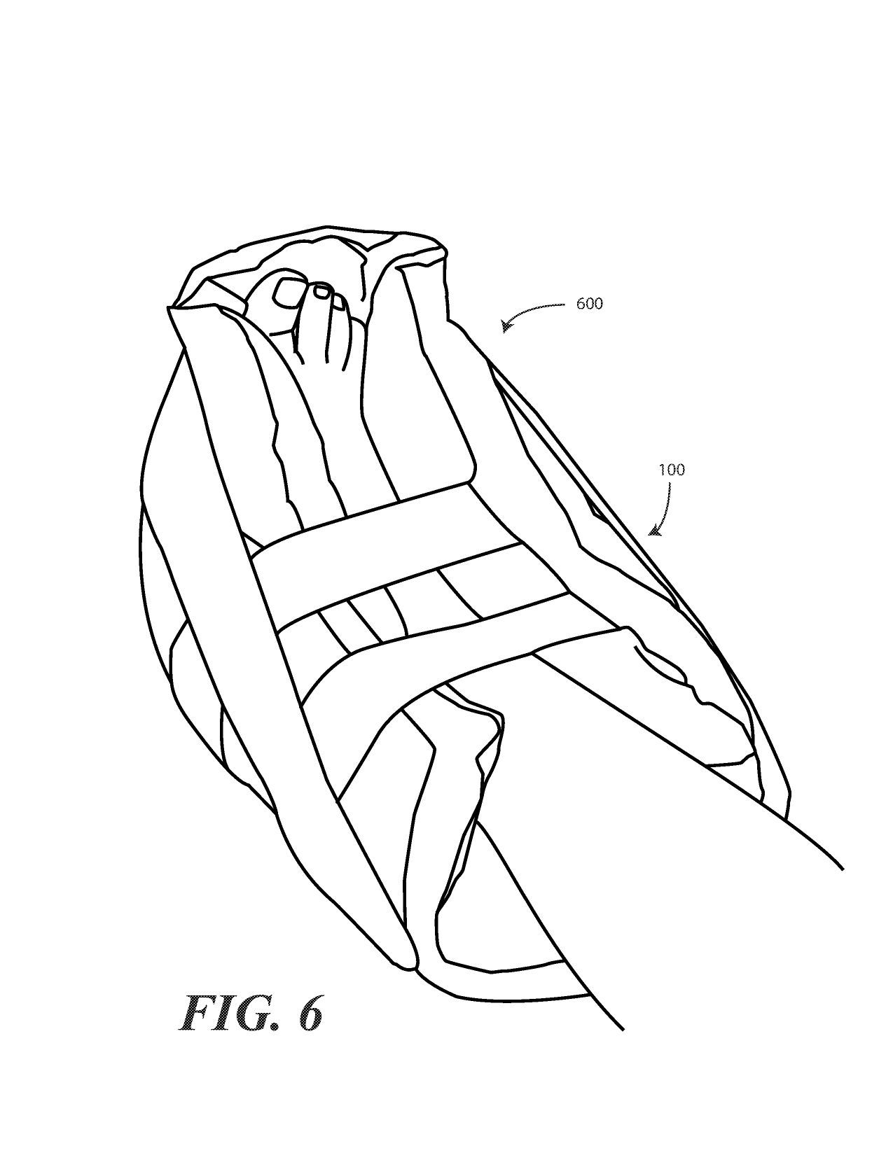

While the method shown in FIGS. 3 and 4 is one method of applying a heel protector 100 to the patient’s leg 302, embodiments of the disclosure provide advantages over other prior art boots in that they can be applied in differing configurations. Turning now to FIGS. 5 and 6, illustrated therein is an alternate method for applying the heel protector 100 to a patient’s leg.

As shown in FIG. 5, the health care services provider 301 has completed the first two steps described above, namely, after inserting the patient’s leg 302 into the leg insertion aperture 104, fastening strap 108 is wrapped about the leg insertion aperture 104 and attached to panel (113). Next, fastening strap 107 is wrapped about the leg insertion aperture 104 and attached to panel (113) to securely affix the leg engaging section 101 about the patient’s leg 302.

However, rather than crisscrossing fastening straps 105,106, in this illustrative embodiment the health care services provider 301 attaches these fastening straps 105,106 to the same side from which they emanate. This is referred to as the “footdrop” protection method. Illustrating by example, fastening strap 106 is stretched parallel to the leg insertion aperture 104 and is attached to panel 112. The same is done with fastening strap 105. The resulting configuration 600 is shown in FIG. 6.

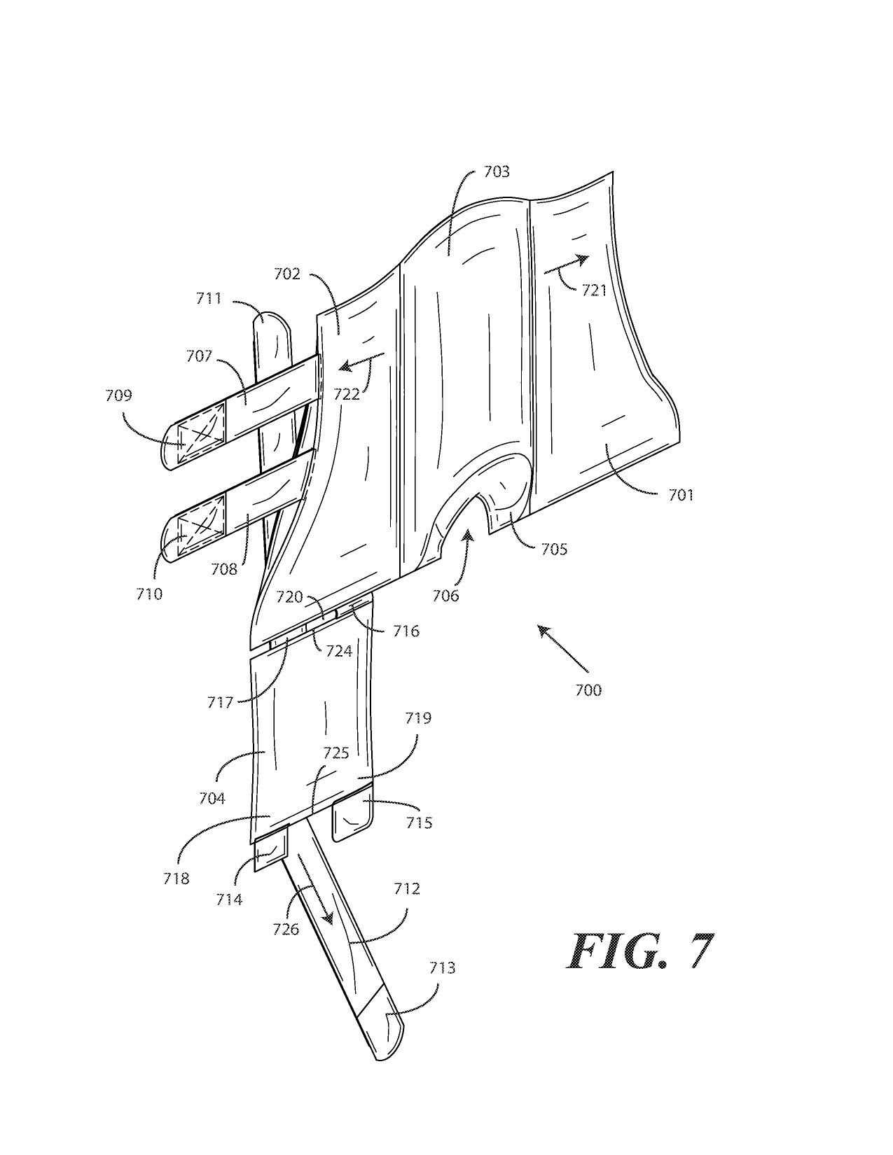

Turning now to FIGS. 7-22, illustrated therein is another heel protector 700 in accordance with one or more embodiments of the disclosure. Embodiments of the disclosure contemplate that while heel protectors such as the heel protector (100) of FIGS. 1-6 work well in practice, such heel protectors (100) often require complete removal for proper treatment of the foot, which can be inconvenient and time-consuming. Therefore, embodiments of the disclosure contemplate there is a need for a heel protector 700 that allows for selective attachment and detachment of the foot engaging section 704 to side portions of the heel protector 700, examples of which include the first side leg engaging section 701 or second side leg engaging section 702, thereby providing optimal comfort and therapeutic benefits while simplifying treatment procedures.

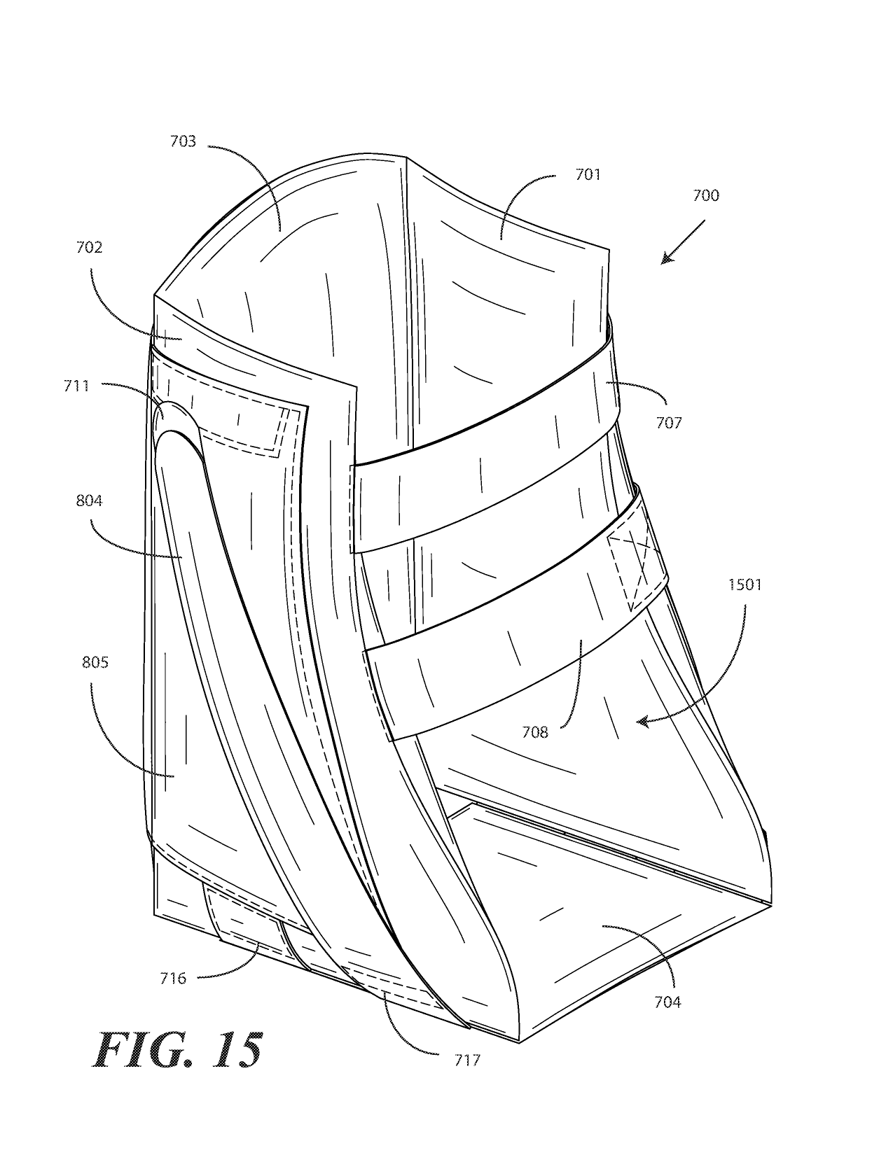

FIGS. 7-14 depict various views of an exemplary heel protector 700 in a substantially planar configuration, with the attachment mechanism of the foot engaging section 704 detached from the first side leg engaging section 701. These figures include perspective, top plan, bottom plan, left elevation, front elevation, and rear elevation views, showcasing the heel protector 700 from multiple angles to provide a comprehensive understanding of its design in an unfastened state. FIGS. 15-22 illustrate the heel protector in a wearable configuration, where the attachment mechanism connects the foot engaging section 704 to the first side leg engaging section 701. These figures present first and second perspective views, top plan, bottom plan, right elevation, and front elevation views, demonstrating how the heel protector 700 appears when assembled and ready for use.

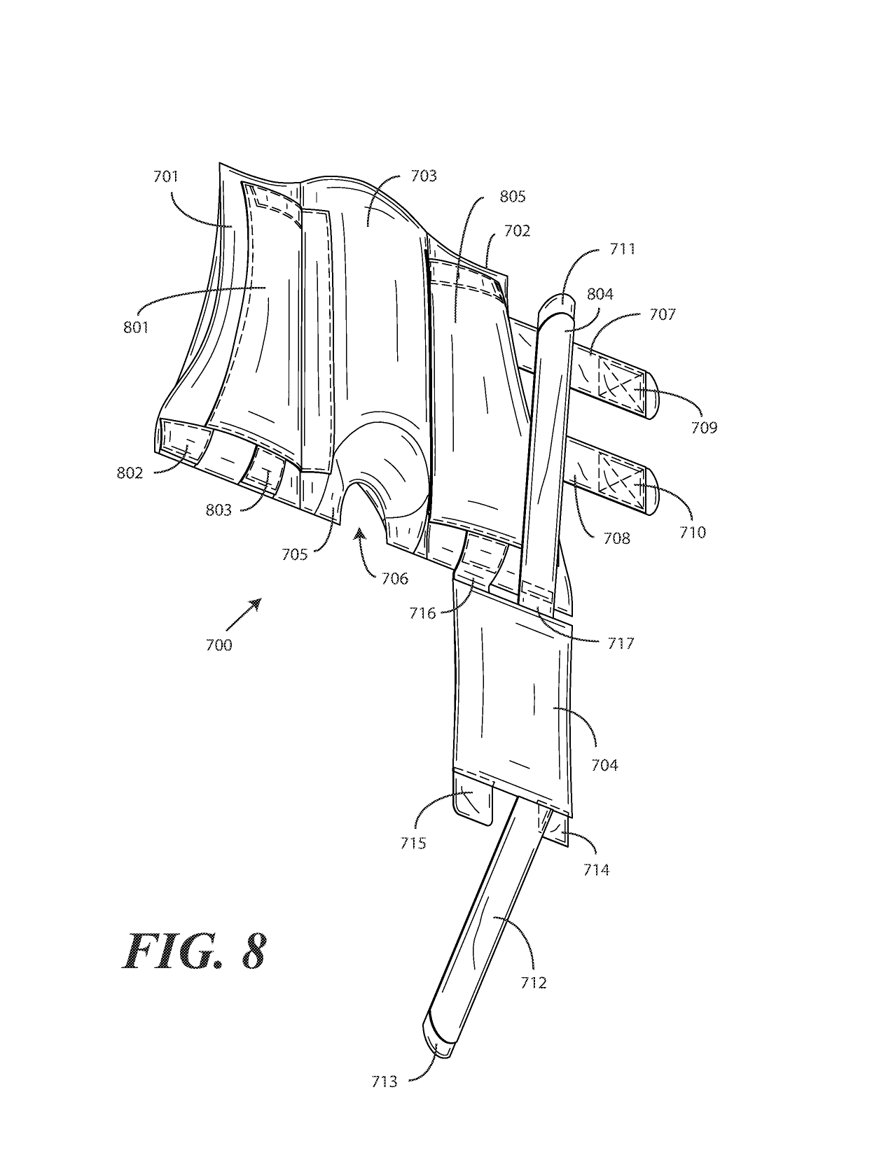

FIG. 7 illustrates a perspective view of a heel protector 700 in a substantially planar configuration, with a first side leg engaging section tab 714 and a second side leg engaging section tab 715 of the foot engaging section 704 shown detached from the first side leg engaging section 701. FIG. 8 shows another perspective view of the heel protector 700 with the first side leg engaging section tab 714 and the second side leg engaging section tab 715 of the foot engaging section 704 of the foot engaging section 704 detached from the first side leg engaging section 701.

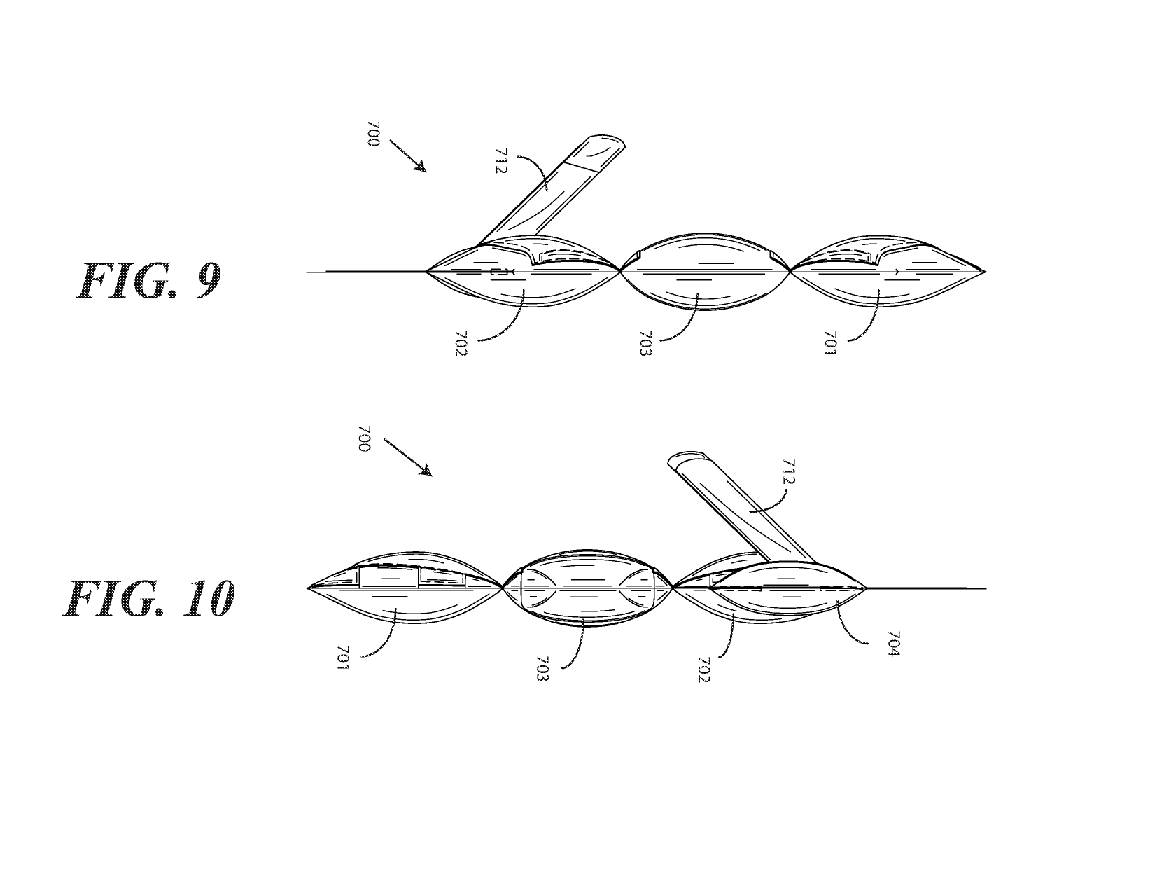

FIG. 9 presents a top plan view of the heel protector 700, with the first side leg engaging section tab 714 and the second side leg engaging section tab 715 of the foot engaging section 704 of the foot engaging section 704 detached from the first side leg engaging section 701. FIG. 10 depicts a bottom plan view of the heel protector 700, with the first side leg engaging section tab 714 and the second side leg engaging section tab 715 of the foot engaging section 704 of the foot engaging section 704 detached from the first side leg engaging section 701, while FIG. 11 shows a right elevation view of the heel protector 700 with the first side leg engaging section tab 714 and the second side leg engaging section tab 715 of the foot engaging section 704 of the foot engaging section 704 detached from the first side leg engaging section 701.

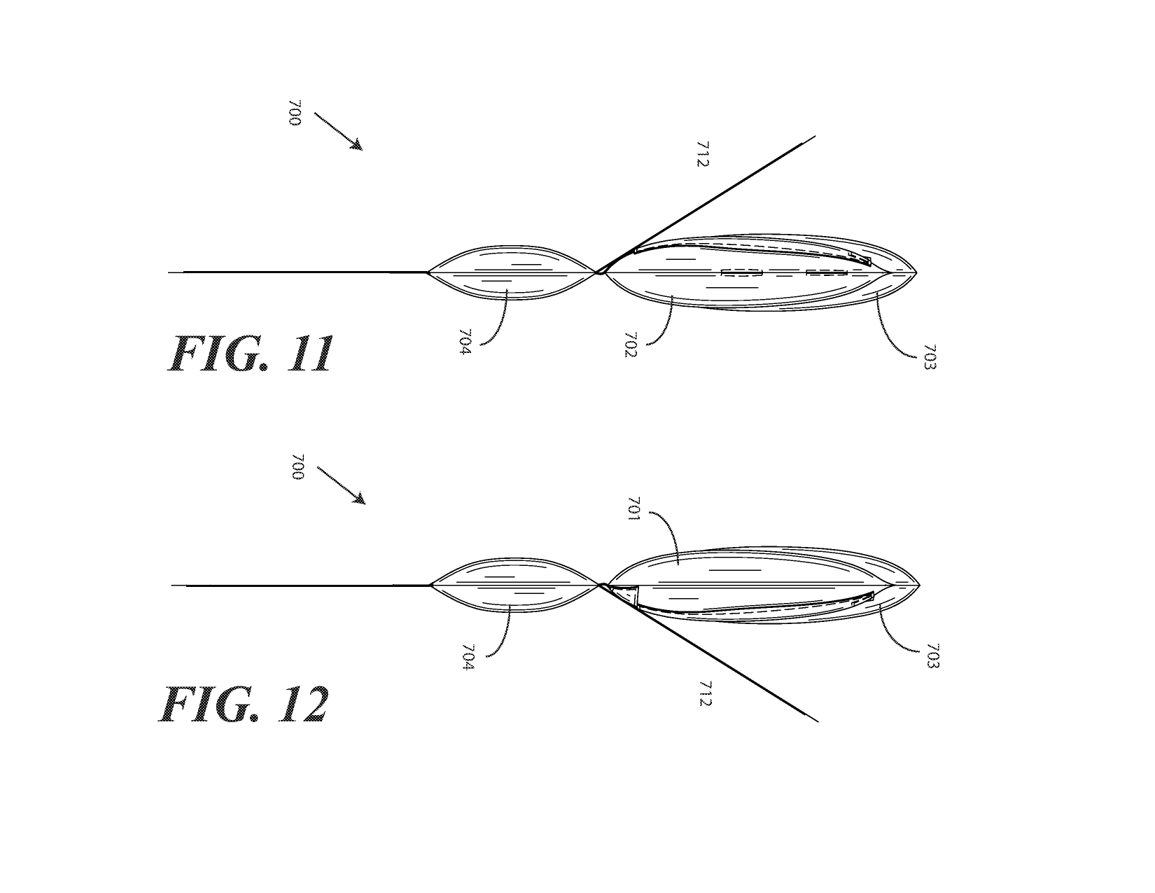

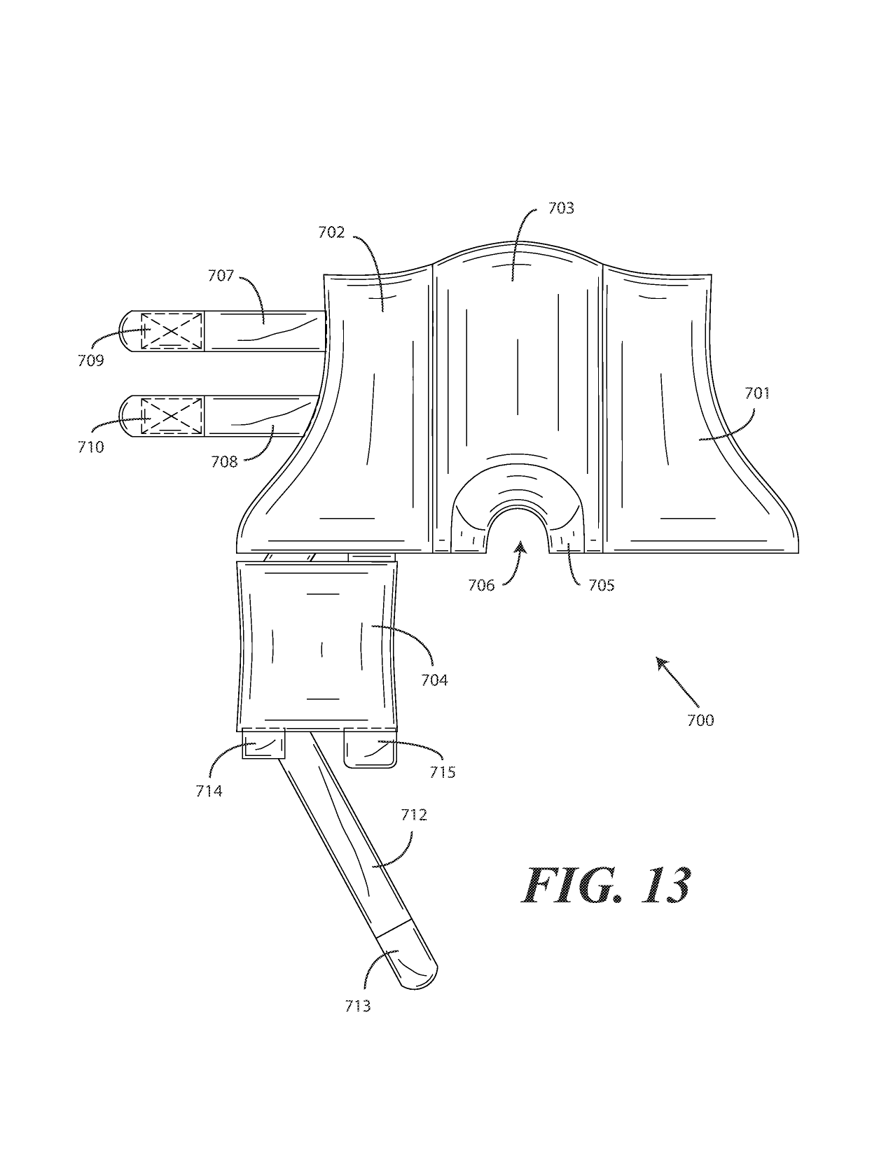

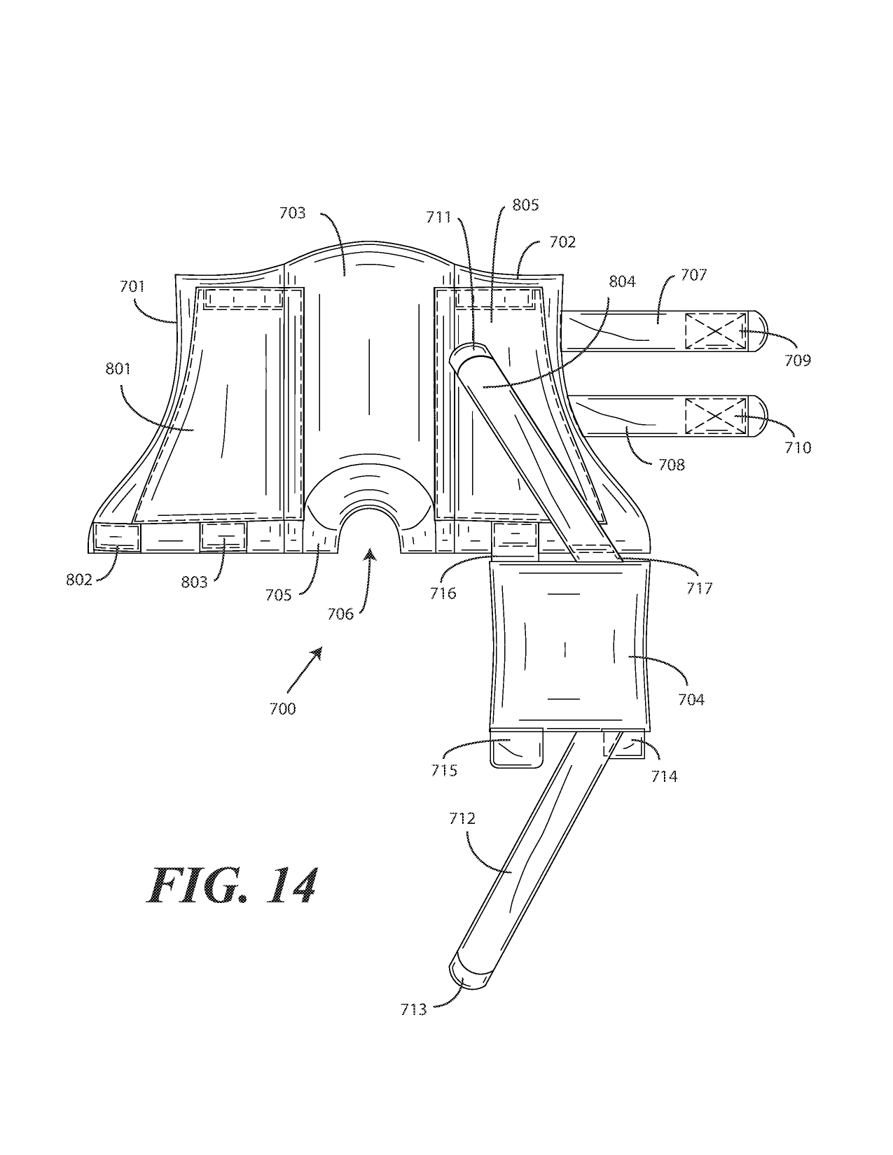

FIG. 12 shows a left elevation view of the heel protector 700, with the first side leg engaging section tab 714 and the second side leg engaging section tab 715 of the foot engaging section 704 of the foot engaging section 704 detached from the first side leg engaging section 701. FIG. 13 presents a front elevation view of the heel protector 700, with the first side leg engaging section tab 714 and the second side leg engaging section tab 715 of the foot engaging section 704 of the foot engaging section 704 detached from the first side leg engaging section 701, while FIG. 14 illustrates a rear elevation view of the heel protector 700 with the first side leg engaging section tab 714 and the second side leg engaging section tab 715 of the foot engaging section 704 of the foot engaging section 704 detached from the first side leg engaging section 701.

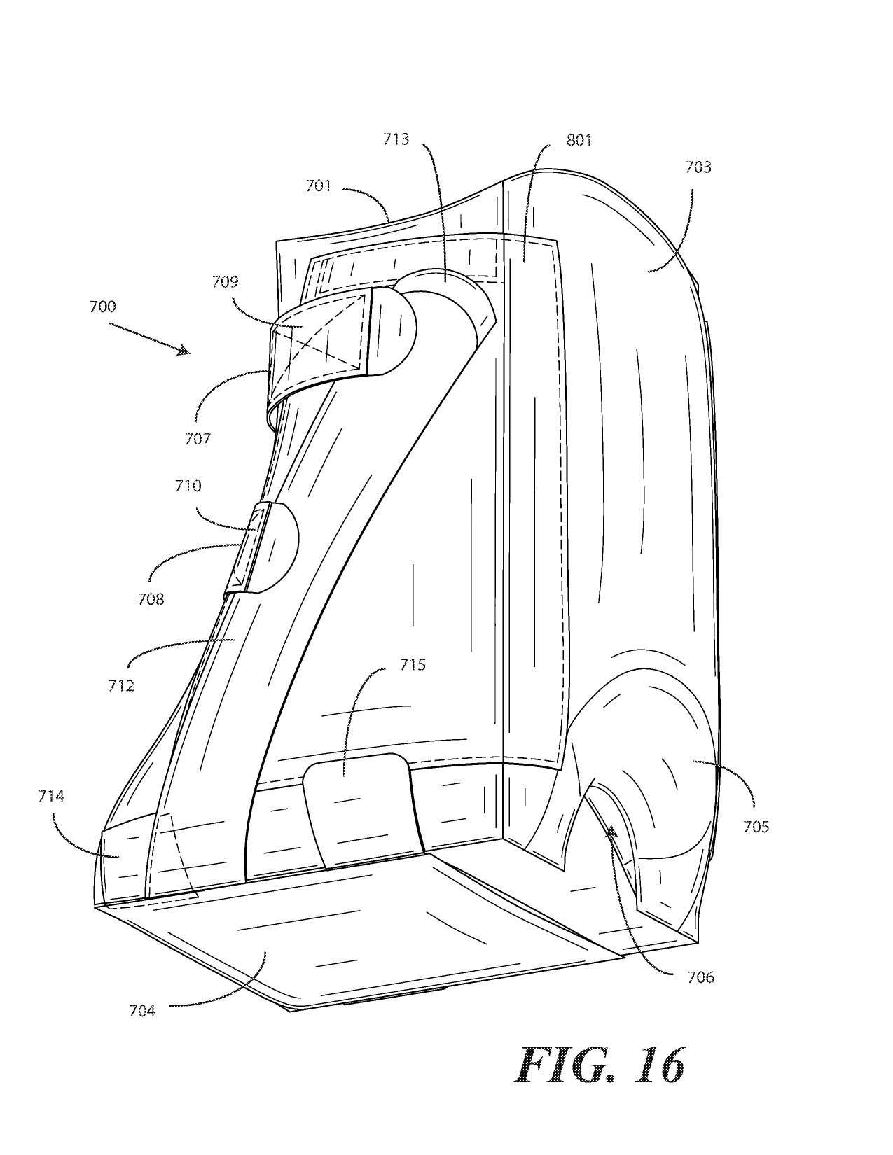

FIG. 15 shows a first perspective view of the heel protector 700 in a wearable configuration, with the first side leg engaging section tab 714 and the second side leg engaging section tab 715 of the foot engaging section 704 of the foot engaging section 704 coupled to the first side leg engaging section 701, while FIG. 16 presents a second perspective view of the heel protector 700 in a wearable configuration, with first side leg engaging section tab 714 and the second side leg engaging section tab 715 of the foot engaging section 704 of the foot engaging section 704 coupled to the first side leg engaging section 701.

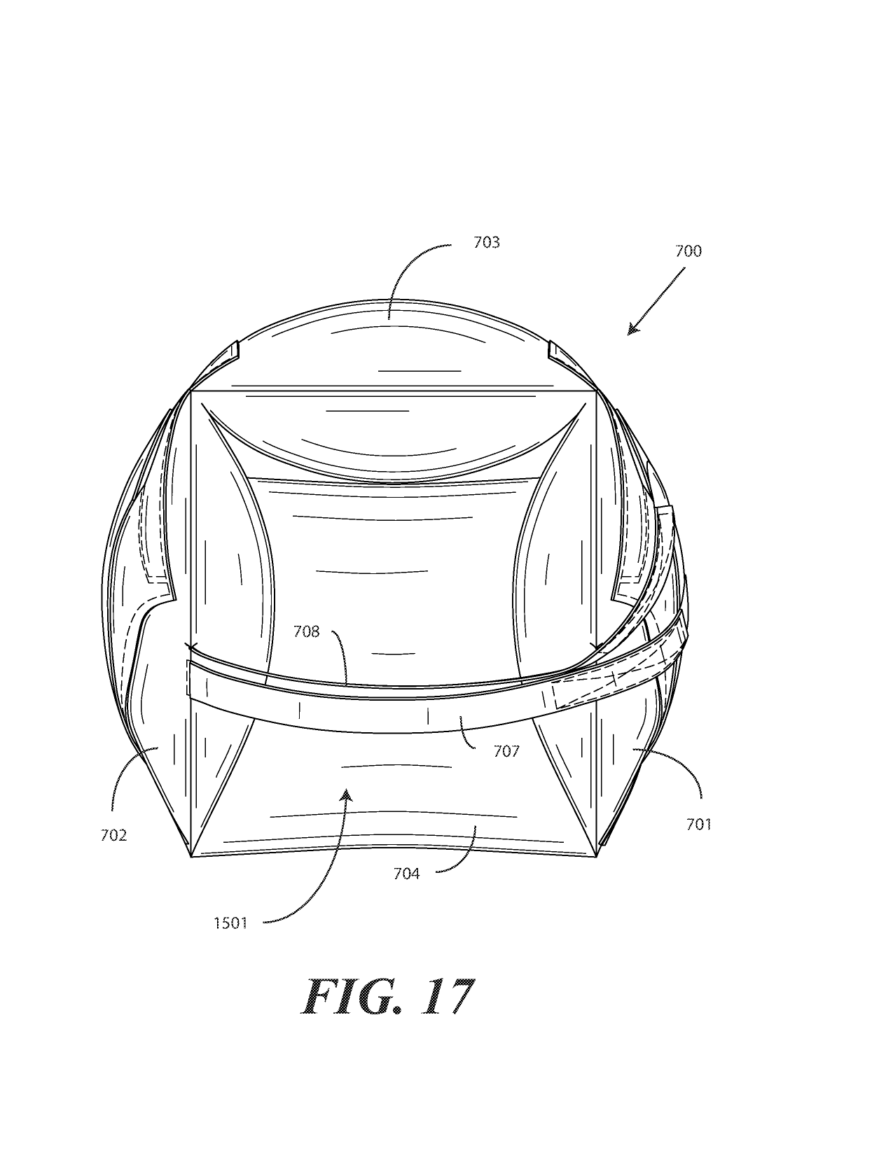

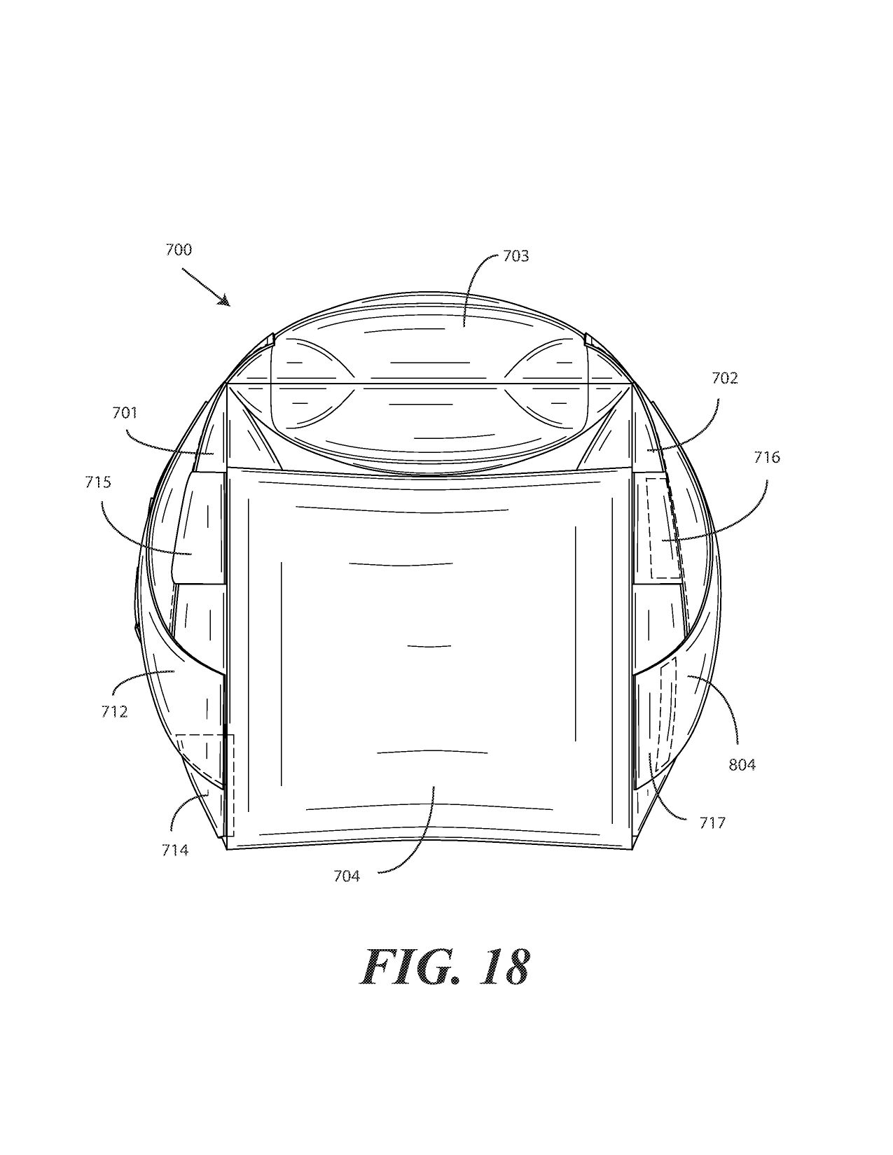

FIG. 17 illustrates a top plan view of the heel protector 700 in a wearable configuration, with the first side leg engaging section tab 714 and the second side leg engaging section tab 715 of the foot engaging section 704 of the foot engaging section 704 coupled to the first side leg engaging section 701, while FIG. 18 depicts a bottom plan view of the heel protector 700 in a wearable configuration, with the first side leg engaging section tab 714 and the second side leg engaging section tab 715 of the foot engaging section 704 of the foot engaging section 704 coupled to the first side leg engaging section 701.

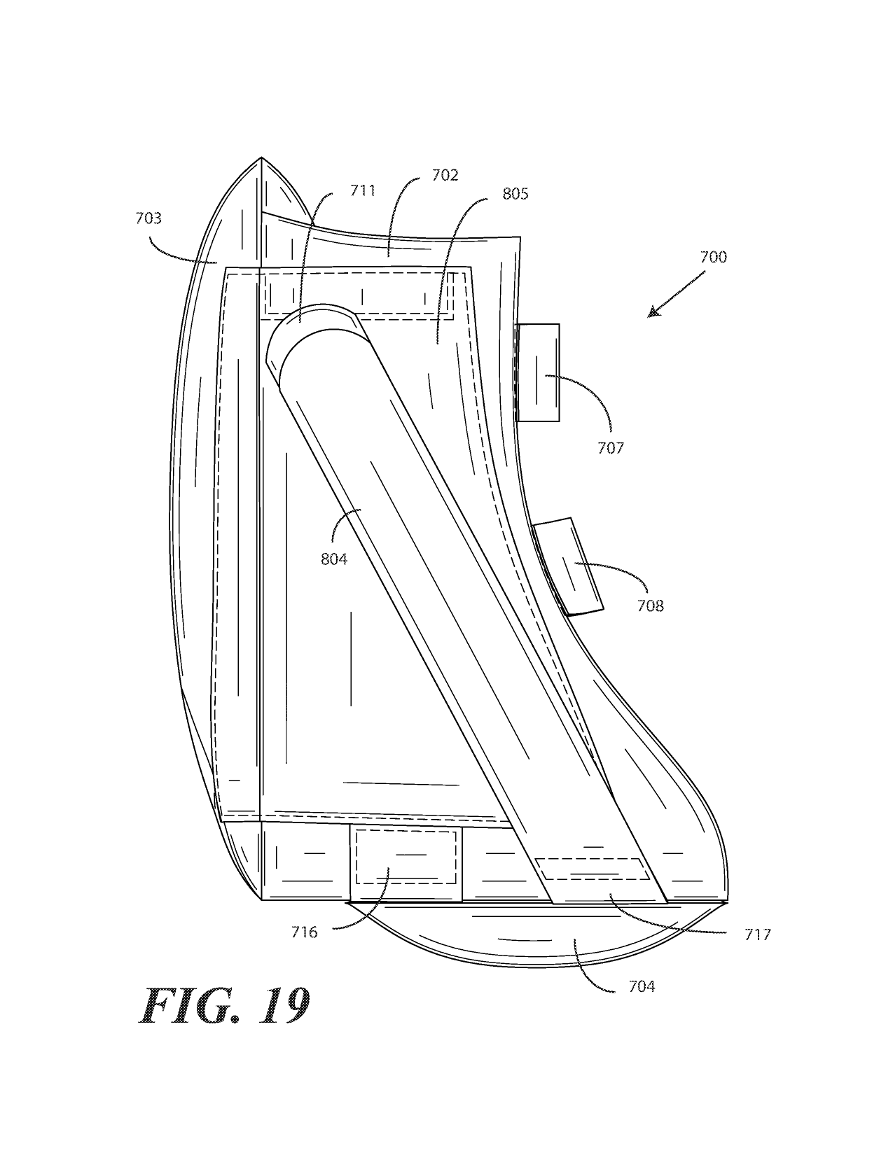

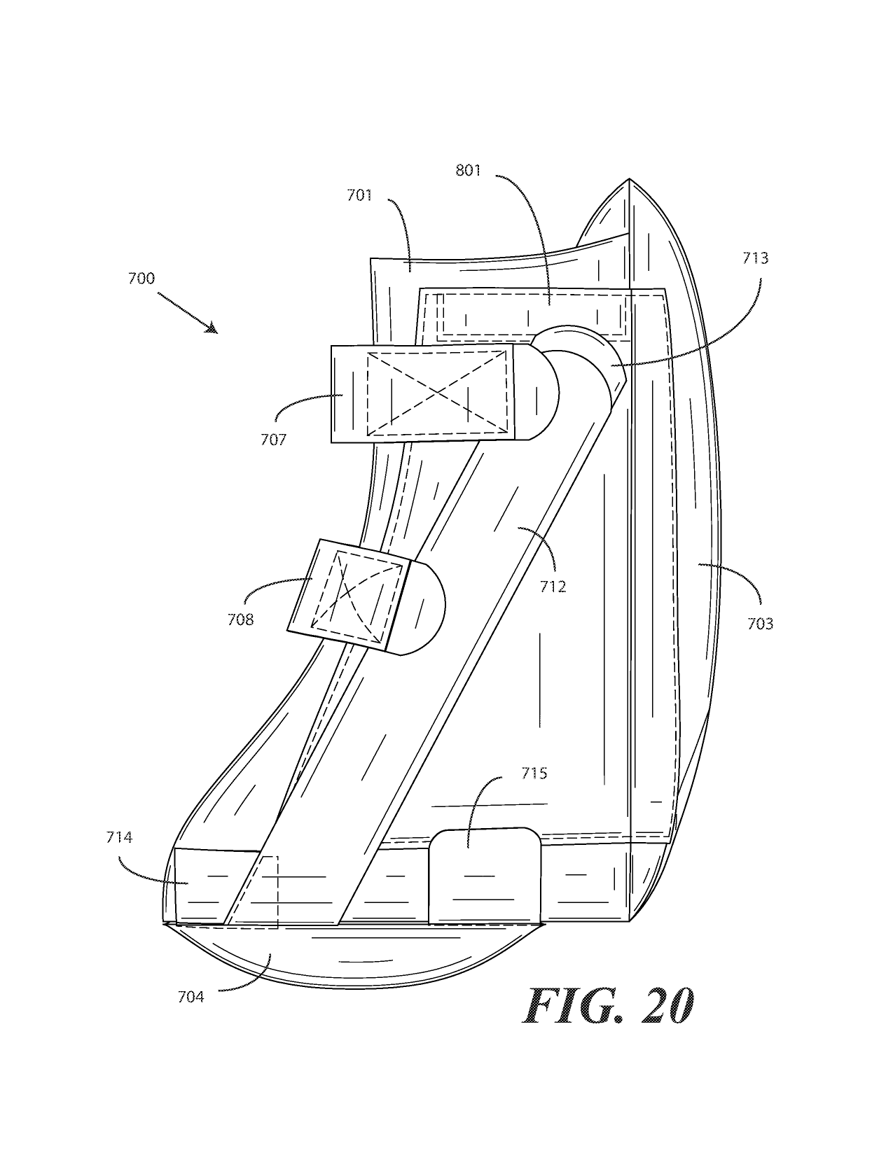

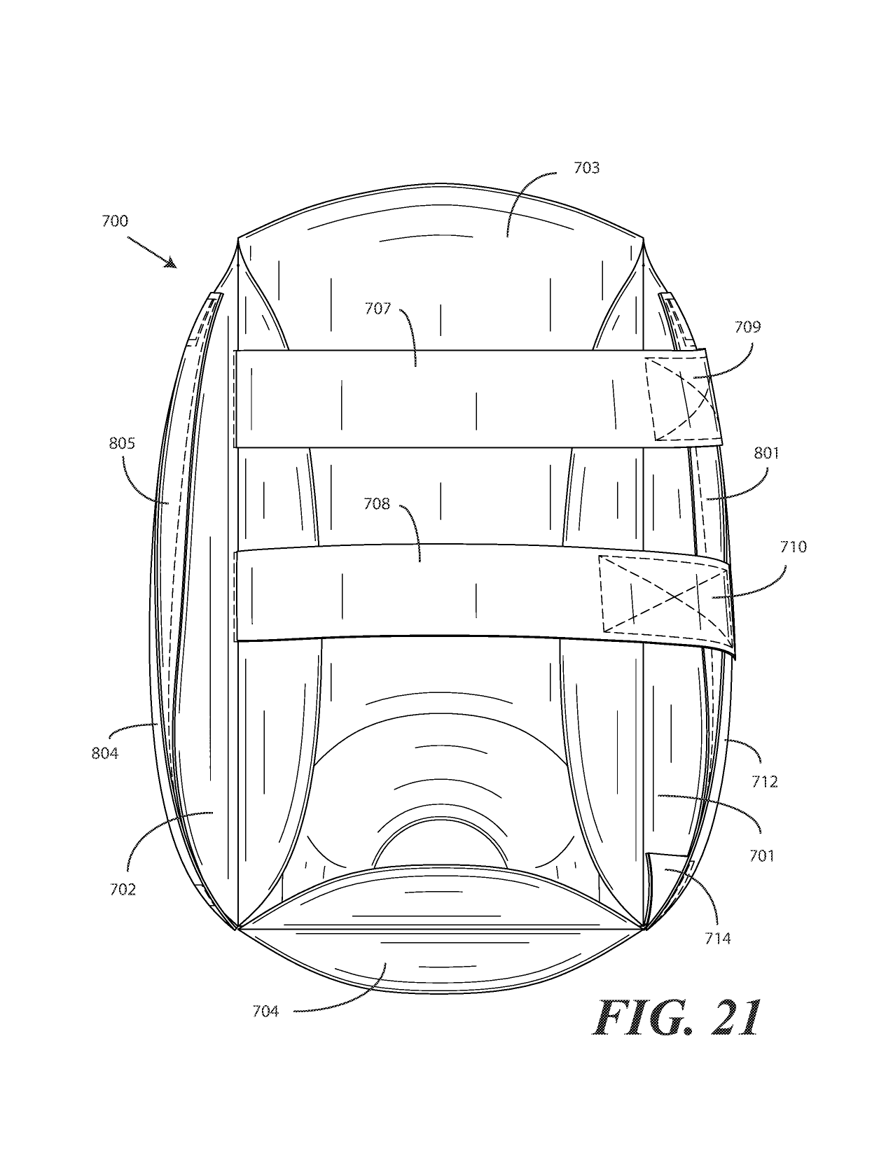

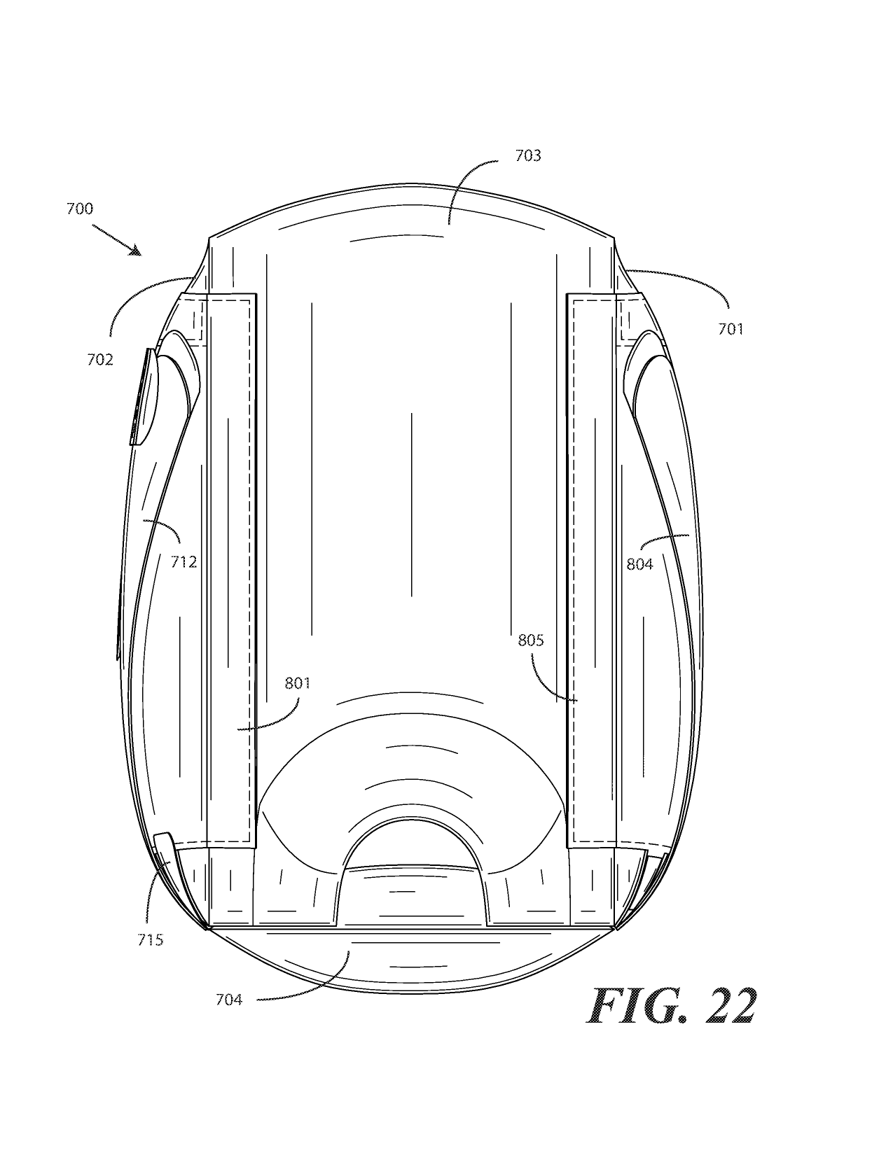

FIG. 19 shows a right elevation view of the heel protector 700 in a wearable configuration, with the first side leg engaging section tab 714 and the second side leg engaging section tab 715 of the foot engaging section 704 of the foot engaging section 704 coupled to the first side leg engaging section 701, while FIG. 20 presents a left elevation view of the heel protector 700 in a wearable configuration, with the first side leg engaging section tab 714 and the second side leg engaging section tab 715 of the foot engaging section 704 of the foot engaging section 704 coupled to the first side leg engaging section 701. FIG. 21 illustrates a front elevation view of the heel protector 700 in a wearable configuration, with the first side leg engaging section tab 714 and the second side leg engaging section tab 715 of the foot engaging section 704 of the foot engaging section 704 coupled to the first side leg engaging section 701, while FIG. 22 shows a rear elevation view of the heel protector 700 in a wearable configuration, with the first side leg engaging section tab 714 and the second side leg engaging section tab 715 of the foot engaging section 704 of the foot engaging section 704 coupled to the first side leg engaging section 701.

Beginning with FIGS. 7-14, illustrated therein are perspective views of one explanatory heel protector 700 configured in accordance with one or more embodiments of the disclosure. As shown in these figures, the heel protector 700 is shown in a substantially planar configuration. In one or more embodiments, the leg engaging section is the main body of the heel protector and comprises three side leg engaging sections, namely, the first side leg engaging section 701, the second side leg engaging section 702, and the third side leg engaging section 703. In the illustrative embodiment of FIGS. 7-14, the first side leg engaging section 701 defines the right side 721 of the leg engaging section, while the second side leg engaging section 702 defines the left side 722 of the leg engaging section of the heel protector 700.

In one or more embodiments, the upper edge of the leg engaging section defined by the first side leg engaging section 701, the second side leg engaging section 702, and the third side leg engaging section 703 is convex, while the right side and left side, defined by the first side leg engaging section 701 and the second side leg engaging section 702, respectively, are concave. This particular shape configuration is designed to provide a comfortable and ergonomic fit around the leg.

However, it is important to note that these specific shapes are not limiting, and other shapes can be employed based on the preferences and requirements of the wearer. One of ordinary skill in the art, having the benefit of this disclosure, would recognize various alternative shapes that could be utilized. Illustrating by example, in other embodiments the first side leg engaging section 701 and the second side leg engaging section 702 are flat while the third side leg engaging section 703 is convex, and so forth.

In still other embodiments, the upper edge could be concave or straight, and the right side and left side could be convex or straight. Additionally, other variations such as asymmetrical shapes or custom contours could be implemented to accommodate specific anatomical considerations or design preferences. The flexibility to adapt the shape of the leg engaging section allows for customization and optimization of the heel protector to ensure a secure and comfortable fit for different individuals and applications.

As will be shown below in FIGS. 15-22, while these sections can be folded to the substantially planar configuration shown in FIGS. 7-14 when the foot engaging section 704 is selectively decoupled from the first side leg engaging section 701, as will be shown in FIGS. 15-22, in other embodiments these sections can fold into substantially orthogonal angles relative to each other so as to wrap around the leg, providing support and protection. The foot engaging section 704 can then be coupled to the first side leg engaging section 701 to retain the heel protector 700 in the wearable configuration.

In one or more embodiments, attached to one of the side leg engaging sections is the foot engaging section 704. In this illustrative embodiment, the foot engaging section 704 is attached to the second side leg engaging section 702. However, in other embodiments, the foot engaging section 704 could be attached to the first side leg engaging section 701.

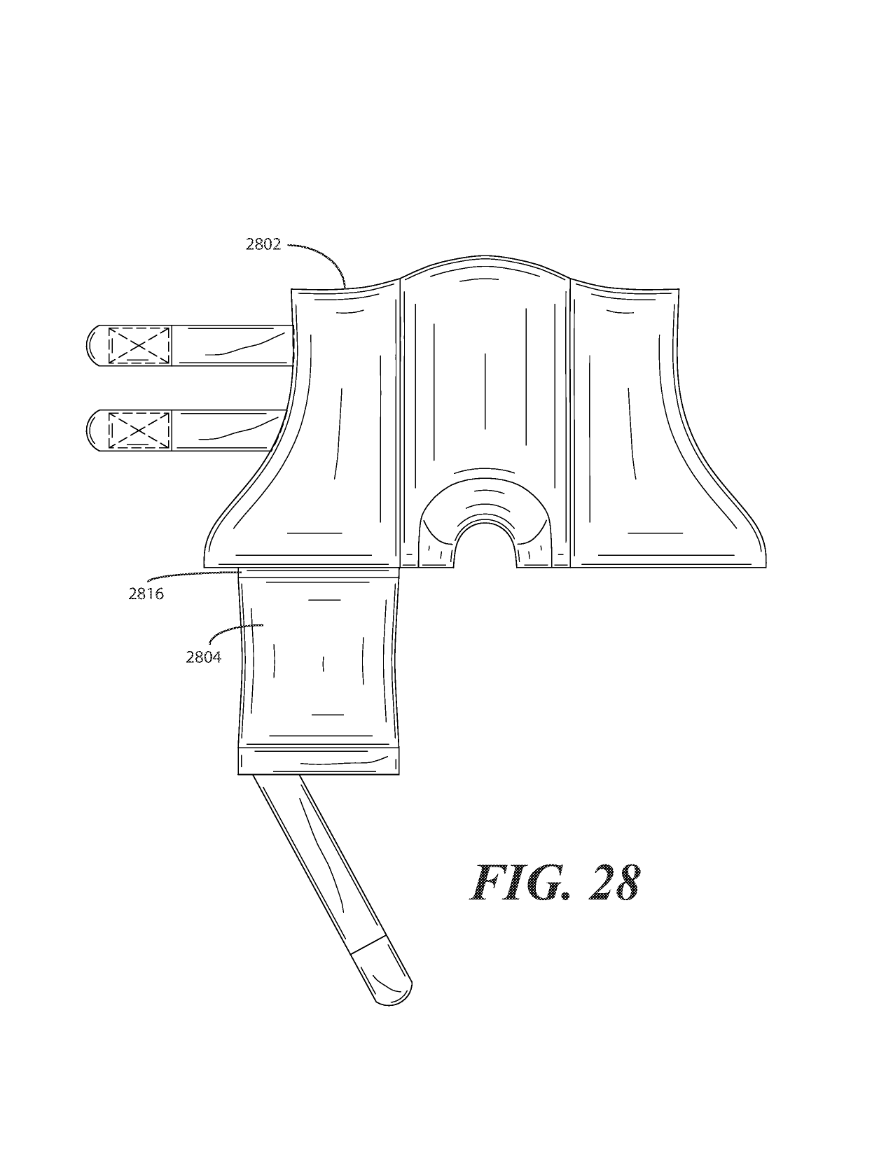

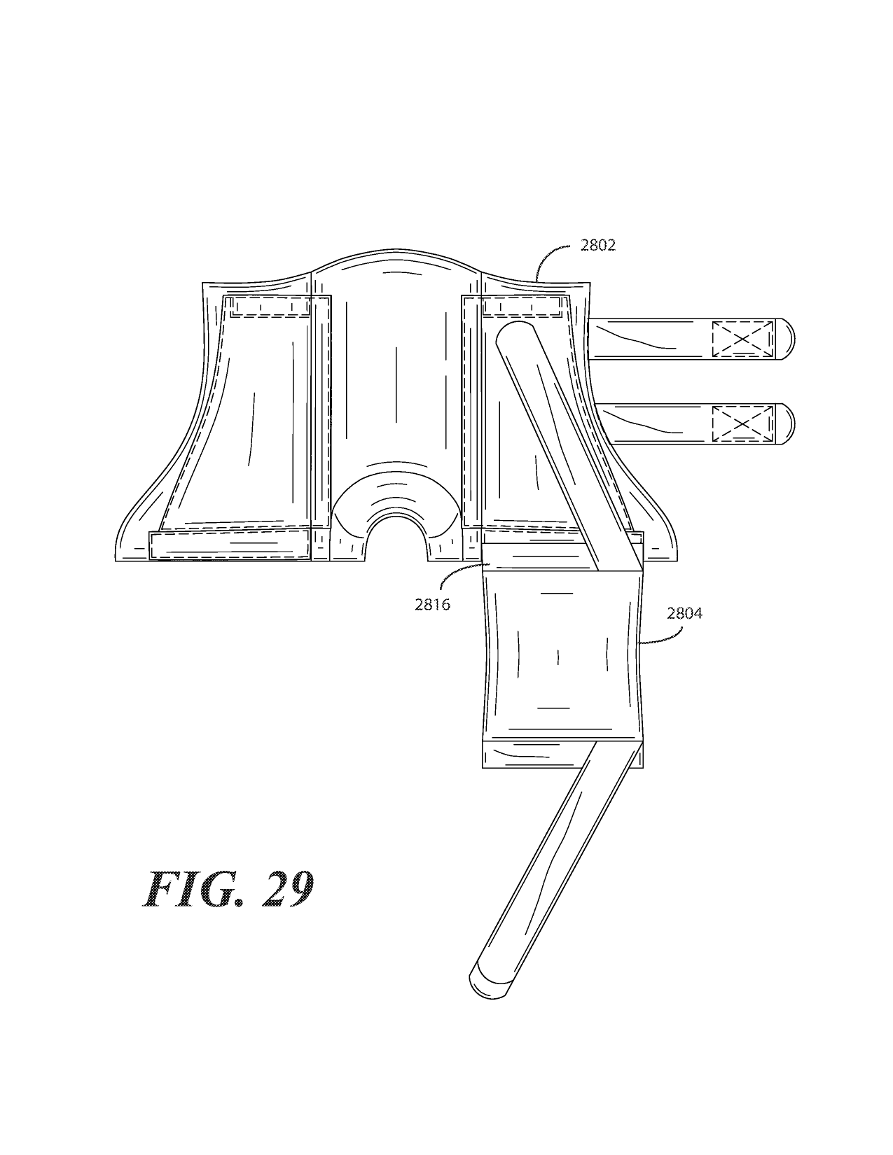

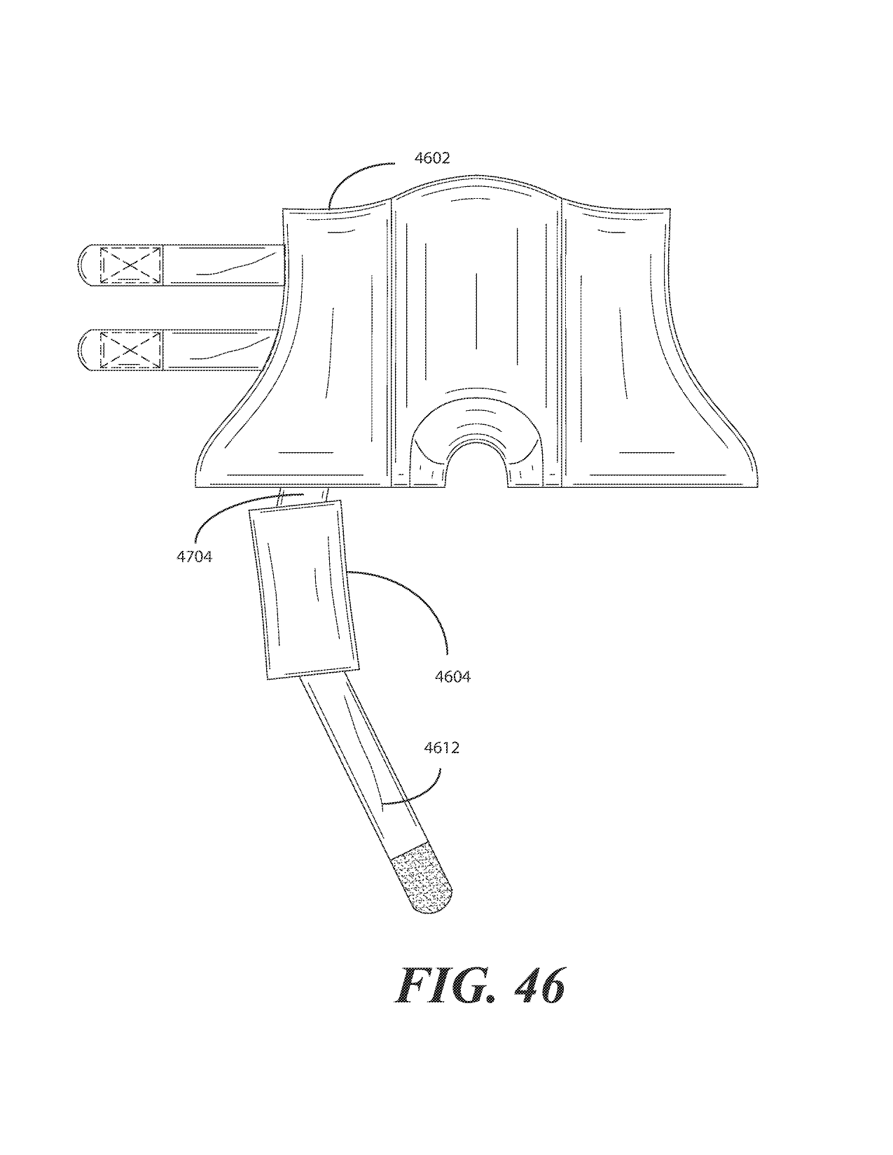

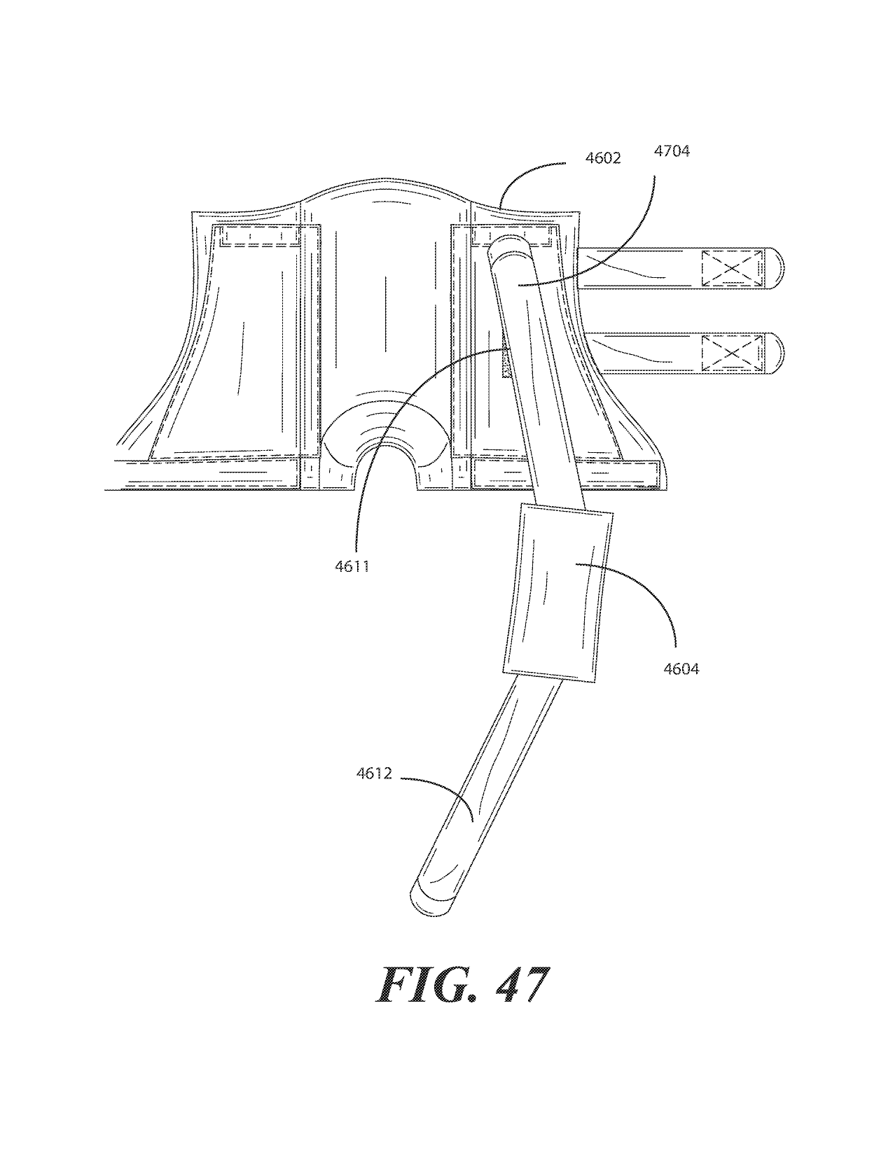

In the illustrative embodiment of FIGS. 7-14, the foot engaging section 704 is stitched to the second leg engaging section 702 by a first side leg engaging section tab 716 and a second side leg engaging section tab 717. However, as shown in FIGS. 28-29, in other embodiments a single side leg engaging section tab 2816 can be used to attach the foot engaging section 2804 to the second leg engaging section 2802. In still other embodiments, such as shown in the embodiment of FIGS. 46-47, the foot engaging section 4604 can be detached from the second leg engaging section 4602 and instead tethered thereto by side leg engaging strap 4612.

Turning now back to FIGS. 7-14, in the embodiment illustrated therein the distal edge of the foot engaging section 704 aligns with a distal end of the second leg engaging section 702 situated away from the third side leg engaging section 703. This alignment ensures that the foot engaging section 704 extends distally to provide a secure fit around the patient's foot, while the second leg engaging section 702 wraps around the leg to offer support and stability. It also offers additional space within which the heel can fit within the heel receiver 705. The configuration additionally allows the foot engaging section 704 to be selectively attached to the first side leg engaging section 701, creating a leg insertion aperture for easy application and removal of the heel protector.

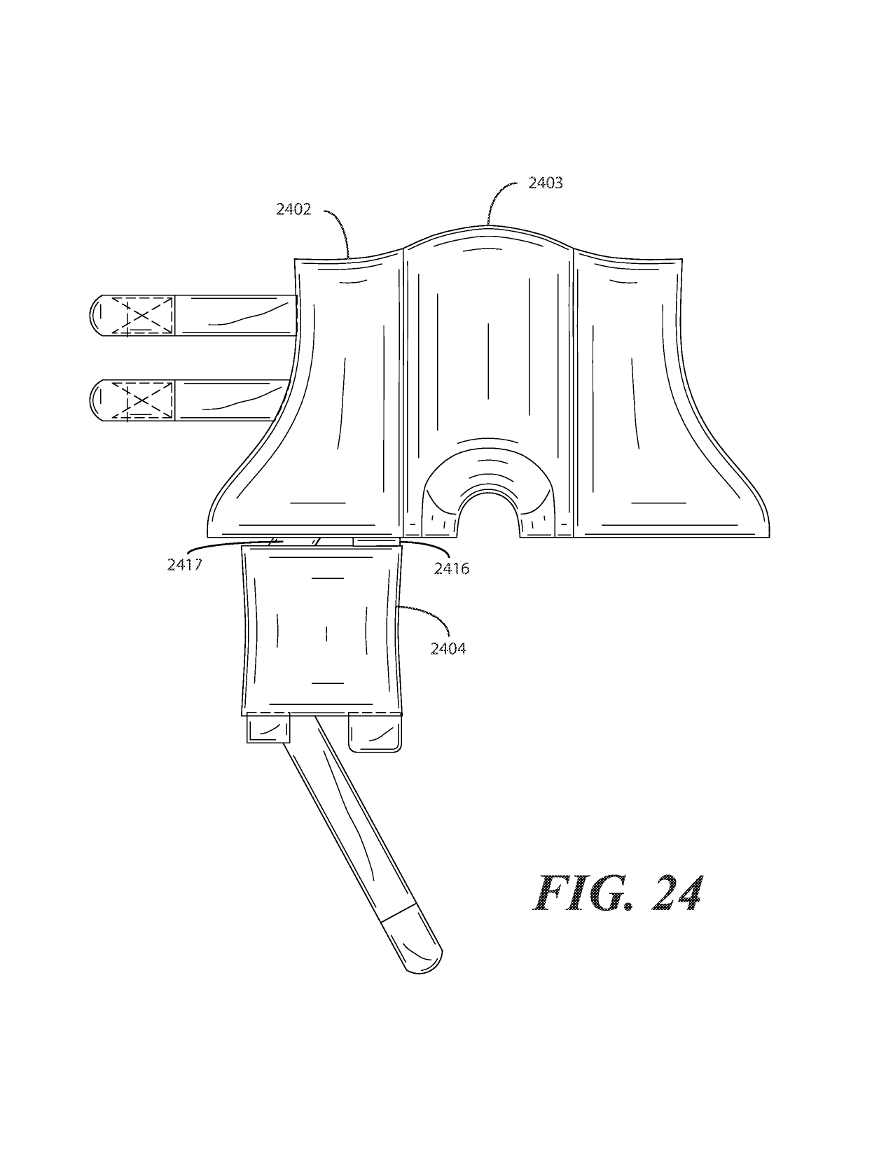

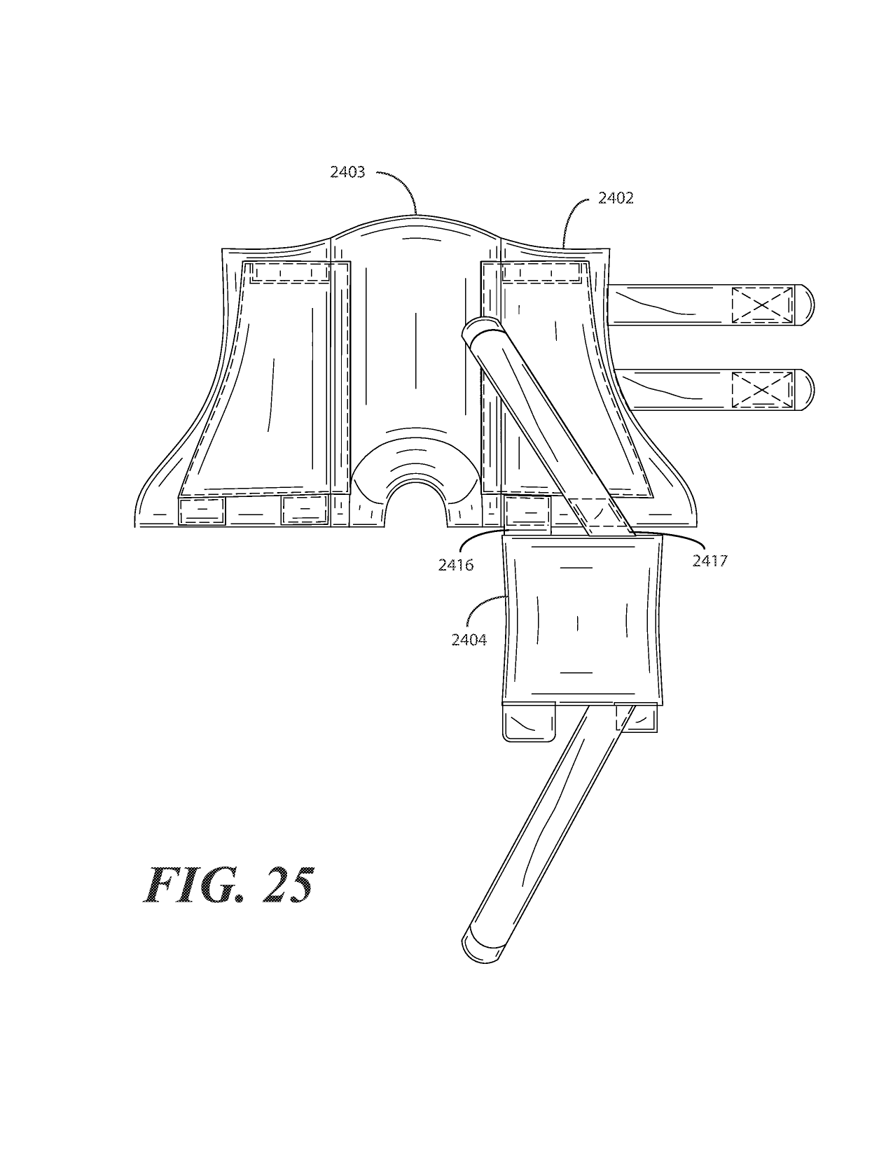

By contrast, the embodiment depicted in FIGS. 24-25 shows the foot engaging section 2404 aligned with a proximal side of the second side leg engaging section 2402. This alignment is defined by the fold line between the second side leg engaging section 2402 and the third side leg engaging section 2403. This configuration allows the foot engaging section 704 to be positioned closer to the third side leg engaging section 2403, providing a different fit and support structure compared to the embodiment of FIGS. 7-14. The proximal alignment offers an alternative method of securing the foot engaging section 704, which may be beneficial for specific therapeutic or comfort requirements.

Other configurations, such as where the foot engaging section is centrally located relative to an axis of the second leg engaging section, will be apparent to those of ordinary skill in the art having the benefit of this disclosure. These alternative alignments provide flexibility in the design and application of the heel protector, allowing for customization based on individual patient needs and preferences. The ability to adjust the position of the foot engaging section relative to the second leg engaging section enhances the versatility and functionality of the heel protector, ensuring optimal support and comfort for various therapeutic applications.

Turning now back to FIGS. 7-14, in this illustrative embodiment the side leg engaging section strap 804 is set back from the distal edge of the second side leg engaging section 702. This configuration allows for a more secure attachment by providing additional material between the strap and the edge, which can help distribute the tension more evenly and reduce the risk of tearing or detachment. The setback position also allows for a more ergonomic fit around the leg, as the design can accommodate the natural curvature of the limb, providing enhanced comfort and stability for the wearer.

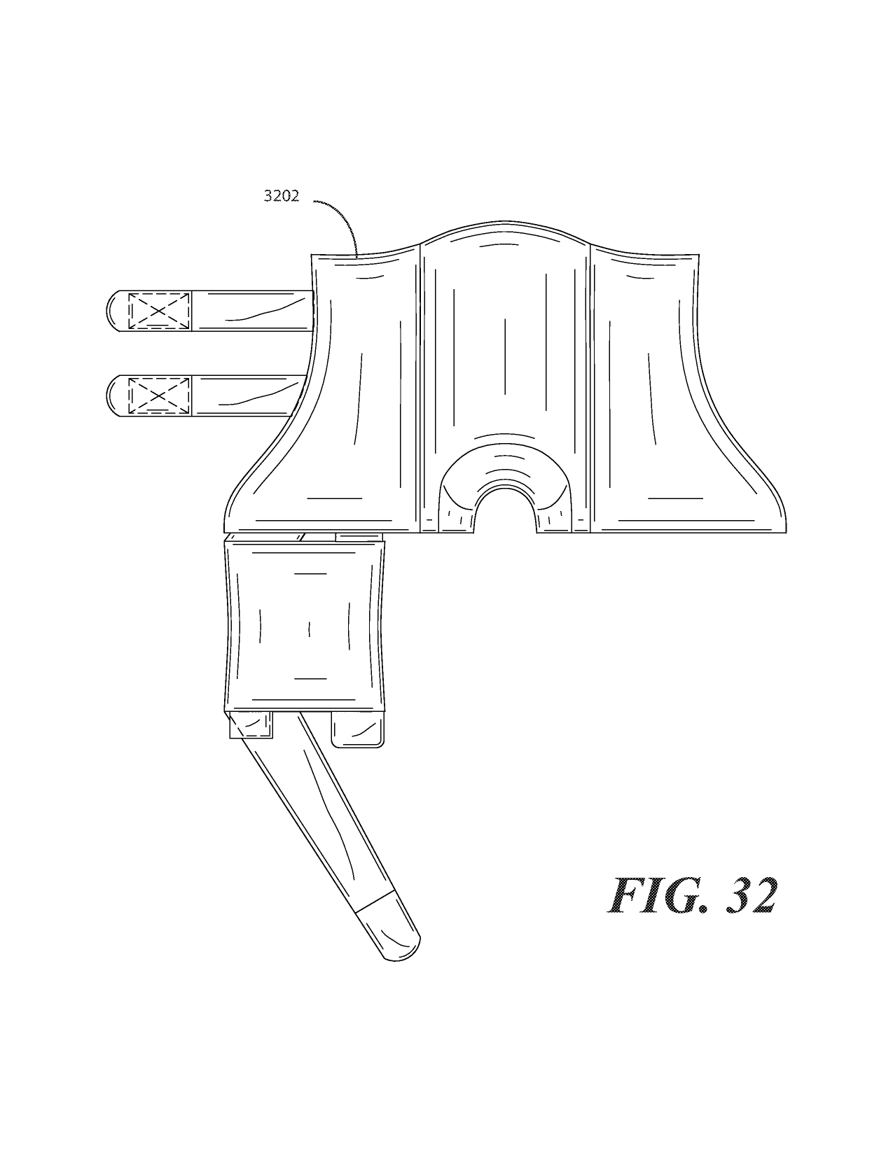

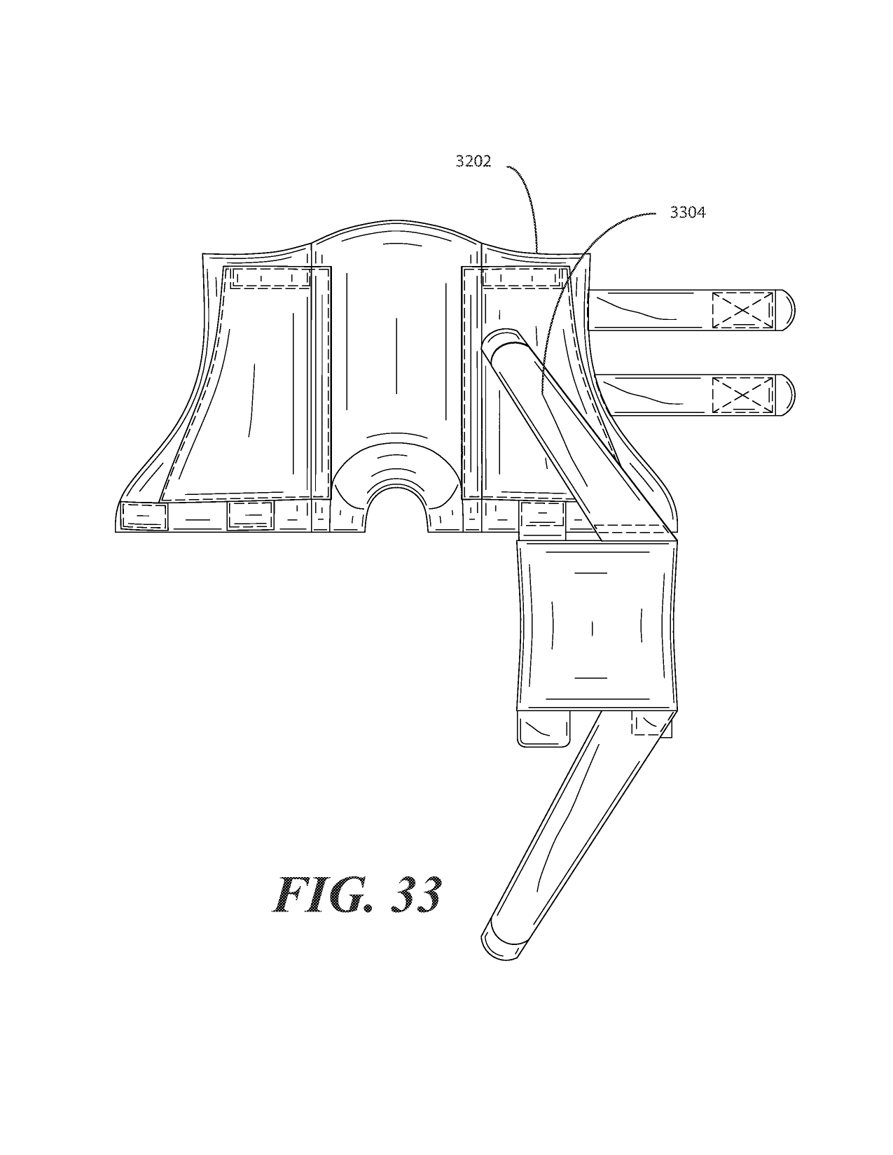

By contrast, in the embodiment shown in FIGS. 32-33, the side leg engaging strap 3304 is aligned with the distal edge of the second side leg engaging section 3202. This alignment can be beneficial in scenarios where a more streamlined design is desired, as the alignment reduces the bulkiness of the heel protector and allows for a closer fit to the leg. Aligning the side leg engaging strap 3304 with the distal edge of the second side leg engaging section 3202 can also simplify the manufacturing process, as the alignment eliminates the need for additional material and stitching to set the strap back from the edge. This design choice can enhance the overall aesthetic of the heel protector, providing a sleeker and more modern appearance.

Moving the side leg engaging strap 3304 to align with the distal edge of the second side leg engaging section 3202 can also facilitate easier and quicker application and removal of the heel protector. The alignment ensures that the side leg engaging strap 3304 is immediately accessible, reducing the time and effort required to secure the heel protector to the leg. This can be particularly advantageous in medical settings where efficiency and ease of use are important. Additionally, the aligned strap position can provide a more consistent and predictable fit, as the aligned strap position eliminates the variability introduced by the setback position, ensuring that the heel protector fits uniformly across different users.

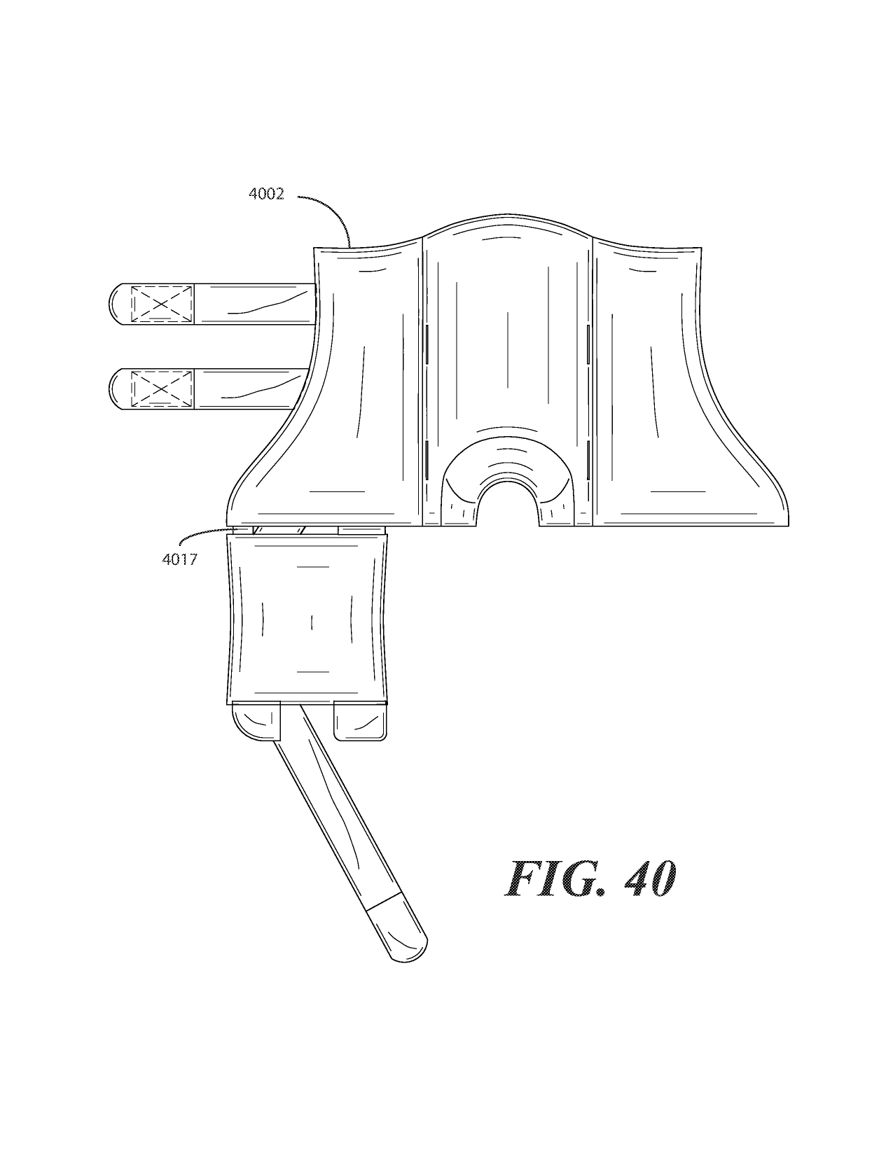

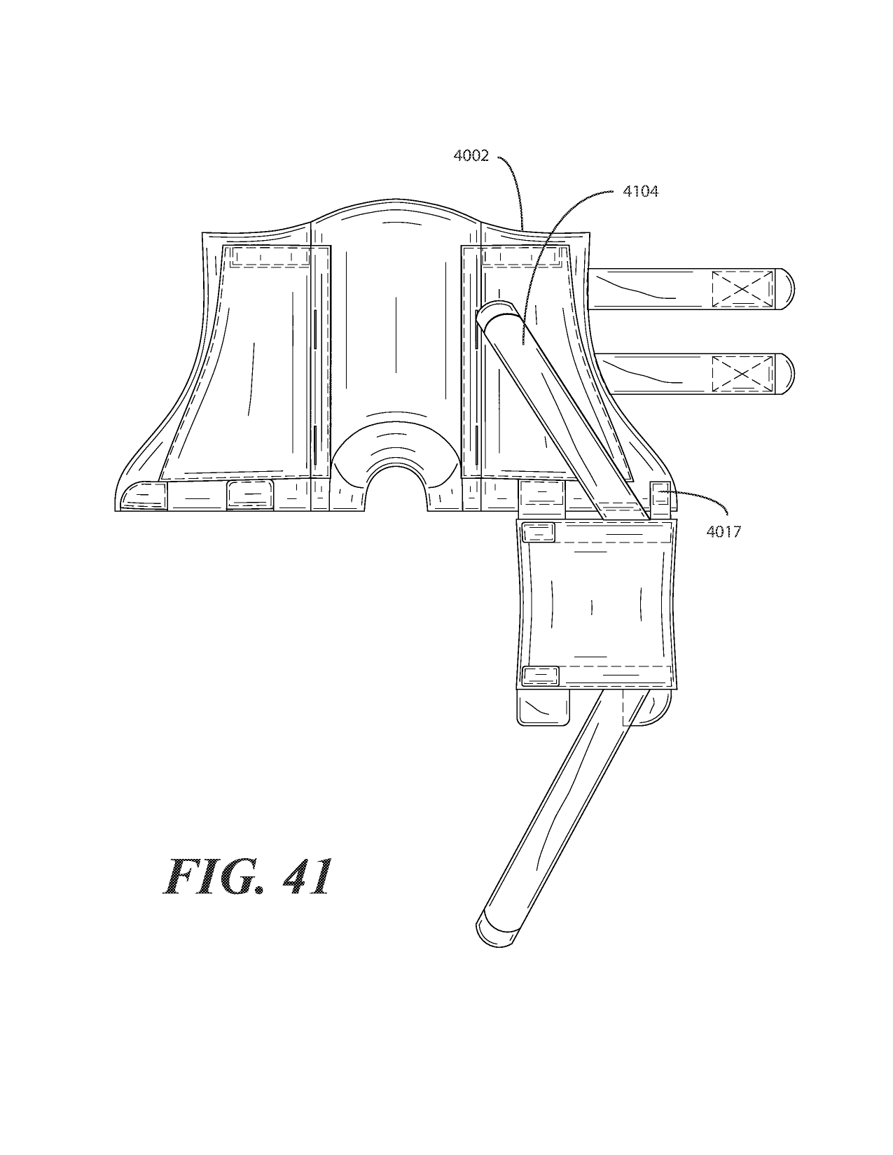

In the embodiment of FIGS. 40-41, the second side leg engaging tab 4017 can be positioned at the distal edge of the second side leg engaging section 4002. This positioning allows the second side leg engaging tab 4017 to extend distally from the second side leg engaging section 4002, providing a secure attachment point for the foot engaging section. The placement of the second side leg engaging tab 4017 at the distal edge ensures that the foot engaging section remains firmly attached to the second side leg engaging section 4002, enhancing the overall stability and support of the heel protector.

Adjacent to, and interior of, the second side leg engaging tab 4017, the side leg engaging strap 4104 is positioned in this illustrative embodiment. This configuration allows the side leg engaging strap 4104 to work in conjunction with the second side leg engaging tab 4017, providing additional reinforcement and security. The proximity of the side leg engaging strap 4104 to the second side leg engaging tab 4017 ensures that the forces exerted on the attachment mechanism are evenly distributed, reducing the risk of detachment or failure. This arrangement enhances the durability and reliability of the heel protector, ensuring that the heel protector remains securely fastened during use.

The positioning of the second side leg engaging tab 4017 at the distal edge of the second side leg engaging section 4002, along with the adjacent placement of the side leg engaging strap 4104, provides a robust and secure attachment mechanism for the foot engaging section. This design ensures that the heel protector offers optimal support and stability, maintaining the effectiveness in providing therapeutic benefits and protection to the patient's heel and leg.

Turning now back to FIGS. 7-14, in this illustrative embodiment the first side leg engaging section tab 716 is longer than the second side leg engaging section tab 717. Moreover, the first side leg engaging section tab 716 is a separate component, while the second side leg engaging section tab 717 is a section of side leg engaging section strap 804. However, other configurations will be obvious to those of ordinary skill in the art having the benefit of this disclosure.

Illustrating by example, in the embodiment shown in FIGS. 24-25, the first side leg engaging section tab 2416 is the same length as the second side leg engaging section tab 2417. Making the first side leg engaging section tab 2416 the same length as the second side leg engaging section tab 2417 can provide several benefits. This uniformity in length ensures a balanced and symmetrical attachment mechanism, which can enhance the overall stability and support of the heel protector. By having both tabs of equal length, the distribution of tension and stress across the attachment points becomes more uniform, reducing the risk of detachment or failure during use. This balanced distribution of forces helps to maintain the proper alignment and positioning of the heel protector, ensuring that the heel protector remains securely fastened to the leg and foot.

Additionally, having the first side leg engaging section tab 2416 and the second side leg engaging section tab 2417 of the same length simplifies the design and manufacturing process. This uniformity reduces the complexity of the attachment mechanism, making the attachment mechanism more intuitive and easier to use for both patients and healthcare providers. The consistent length of the tabs allows for a straightforward and predictable fastening process, minimizing the potential for errors or misalignment during application. This ease of use is particularly advantageous in medical settings where efficiency and accuracy are critical.

Moreover, the equal length of the first side leg engaging section tab 2416 and the second side leg engaging section tab 2417 can contribute to a more aesthetically pleasing design. The symmetrical appearance of the tabs creates a visually balanced and cohesive look, enhancing the overall aesthetic appeal of the heel protector. This can be particularly beneficial for patients who value both functionality and aesthetics in their medical devices. The streamlined design with uniform tab lengths adds to the overall comfort and satisfaction of the wearer, making the heel protector not only effective but also visually appealing.

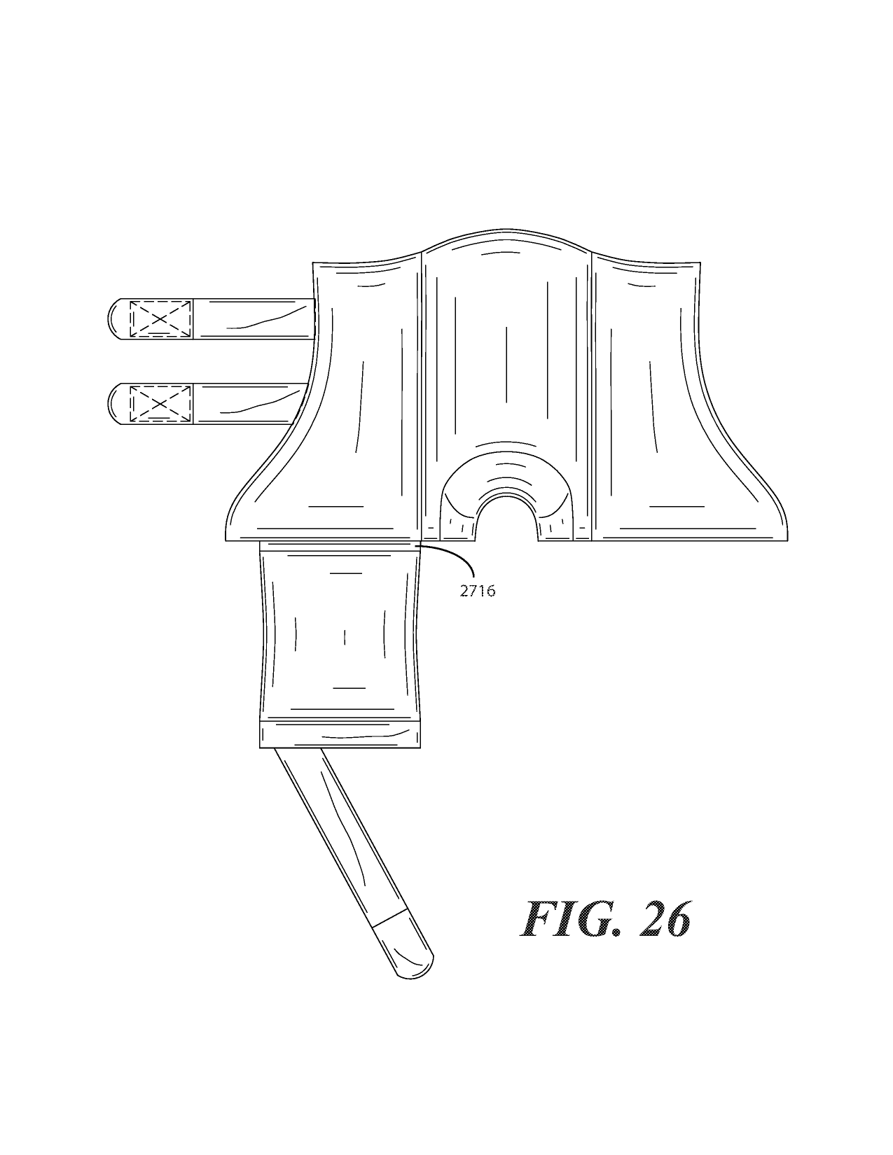

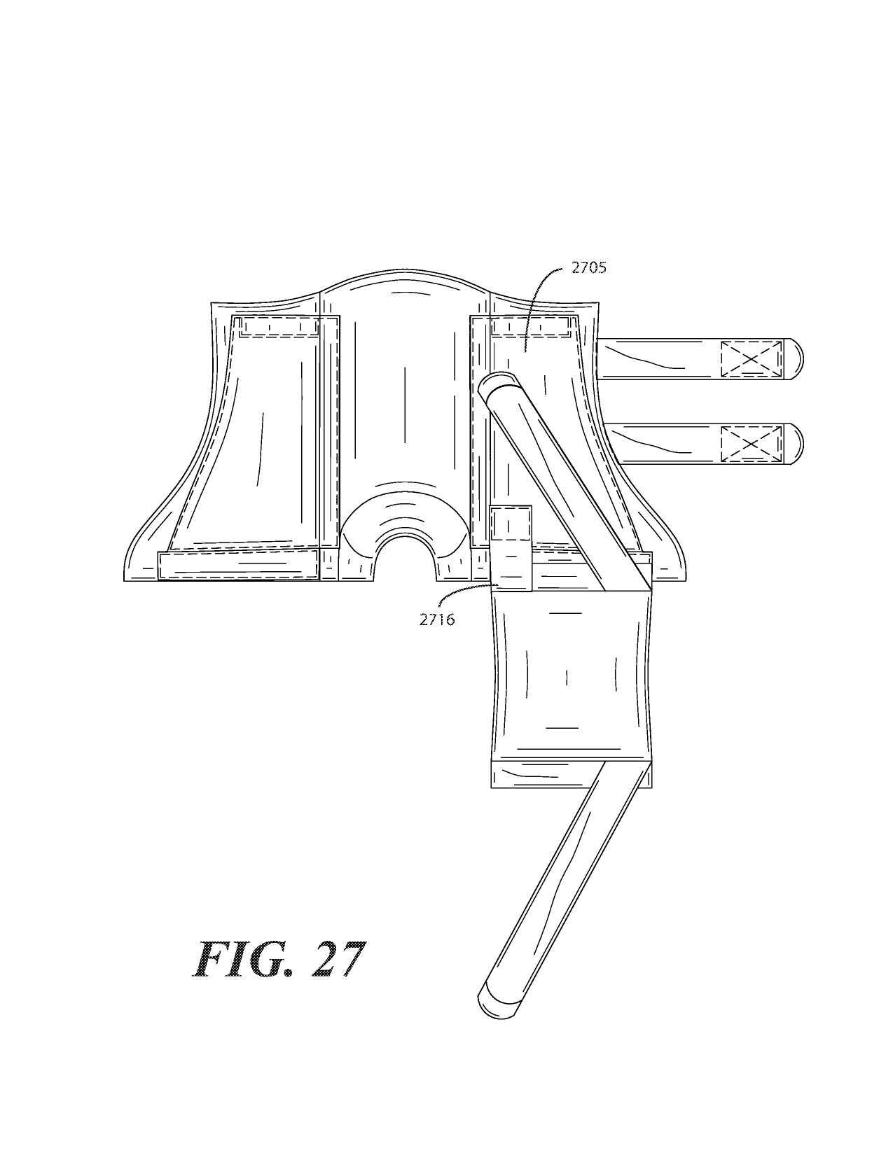

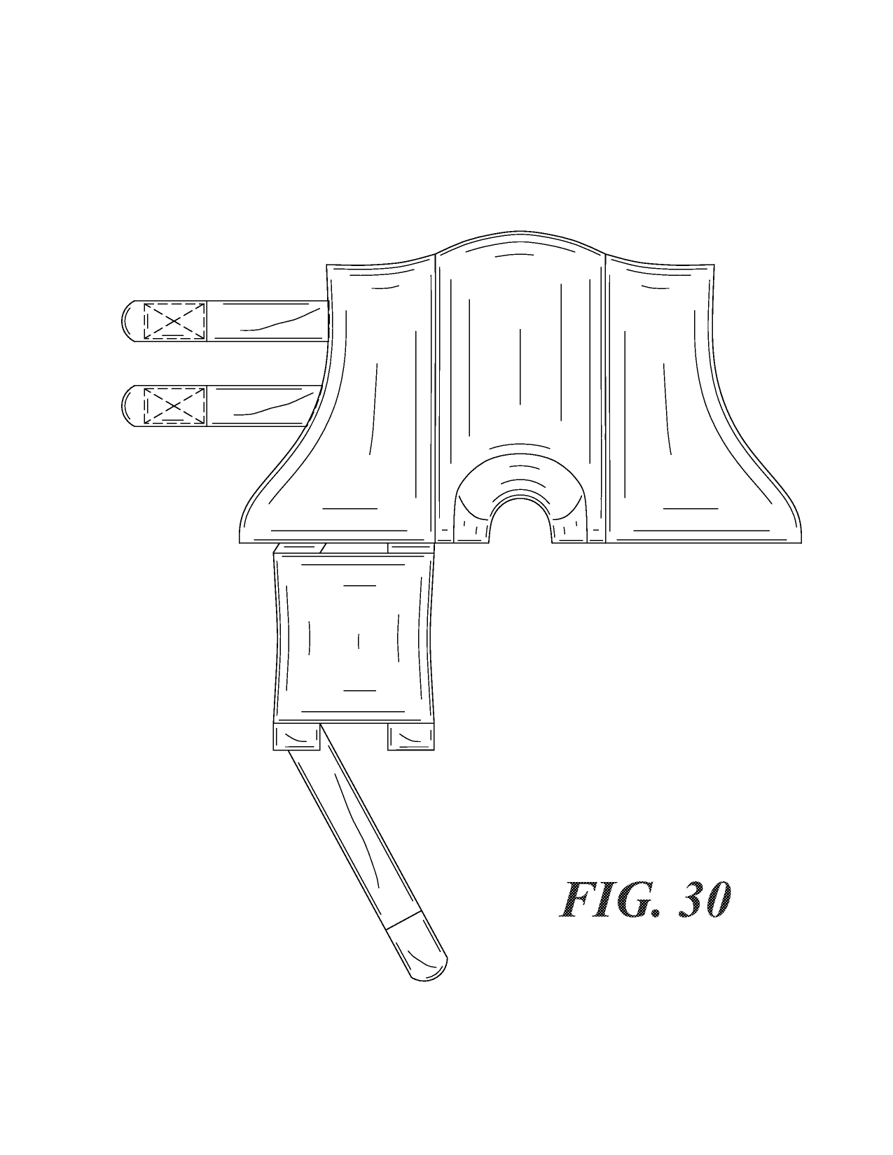

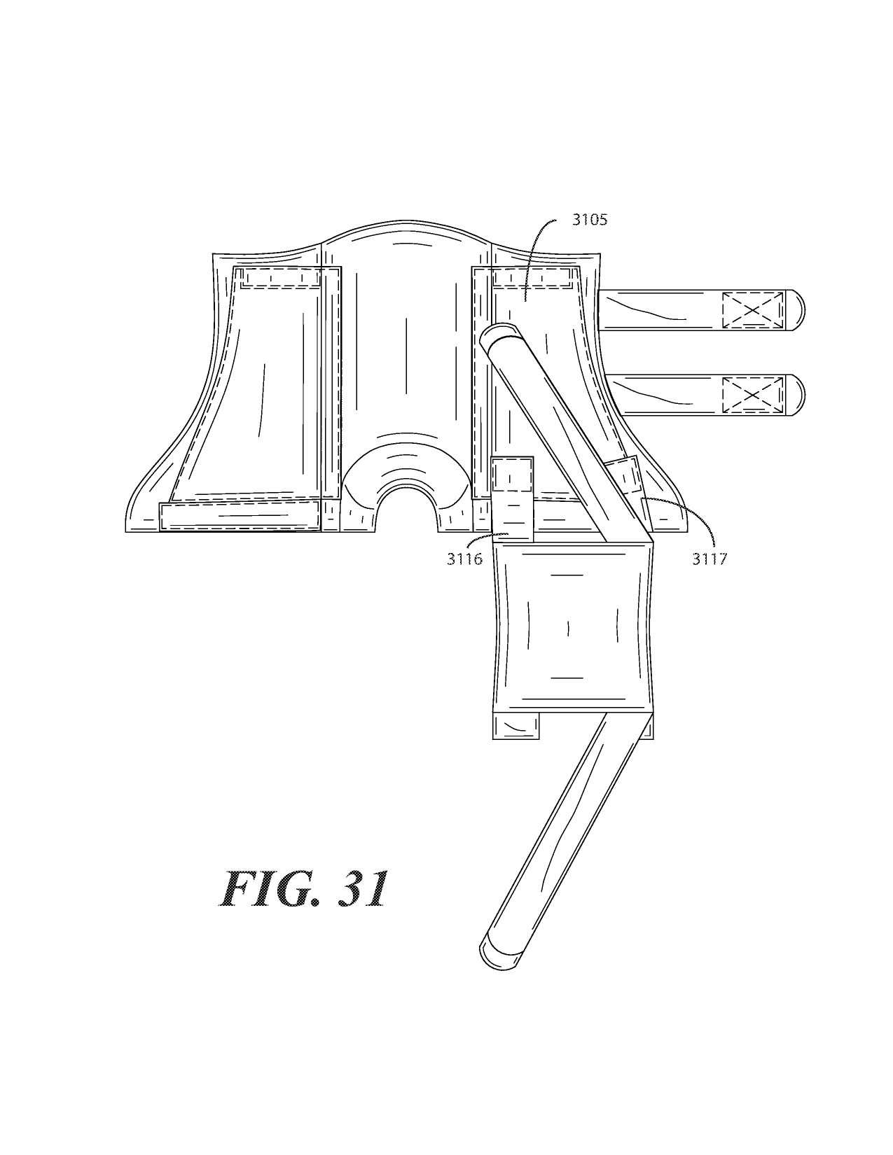

In the embodiment of FIGS. 26-27, the first side leg engaging tab 2716 attaches to the second loop fastener panel 2705. In the embodiment of FIGS. 30-31, the first side leg engaging section tab 3116 and the second side leg engaging section tab 3117 both attach to the second loop fastener panel 3105.

Attaching both the first side leg engaging section tab 3116 and the second side leg engaging section tab 3117 to the second loop fastener panel 3105 provides several benefits. This configuration enhances the overall stability and security of the heel protector by ensuring that both tabs are securely fastened to a common attachment point. By having a single loop fastener panel 3105 to which both tabs attach, the distribution of tension and stress across the attachment mechanism becomes more uniform, reducing the risk of detachment or failure during use.

Additionally, this arrangement simplifies the design and application process of the heel protector. With both tabs attaching to the same loop fastener panel 3105, the user can easily and quickly secure the heel protector in place without the need to align multiple fastener points. This ease of use is particularly advantageous in medical settings where time and efficiency are essential. The single attachment point also minimizes the complexity of the fastening system, making the fastening system more intuitive for users to apply and adjust the heel protector as needed.

Moreover, the use of a single loop fastener panel 3105 for both tabs can enhance the aesthetic appeal of the heel protector. The streamlined design reduces the number of visible fastener points, creating a cleaner and more cohesive appearance. This can be particularly beneficial for patients who value both functionality and aesthetics in their medical devices. The simplified fastening system also contributes to a more comfortable wearing experience, as the simplified fastening system reduces the potential for pressure points or discomfort caused by multiple fastener locations.

Turning now back to FIGS. 7-14, in one or more embodiments the foot engaging section 704 is selectively attachable to one of the first side leg engaging section 701 or the second side leg engaging section 702 and is fixedly attached to another of the first side leg engaging section 701 and/or the second side leg engaging section 702. In the illustrative embodiment of FIGS. 7-14, the foot engaging section 704 is selectively attachable to the first side leg engaging section 701 and is fixedly attached to the second side leg engaging section 702.

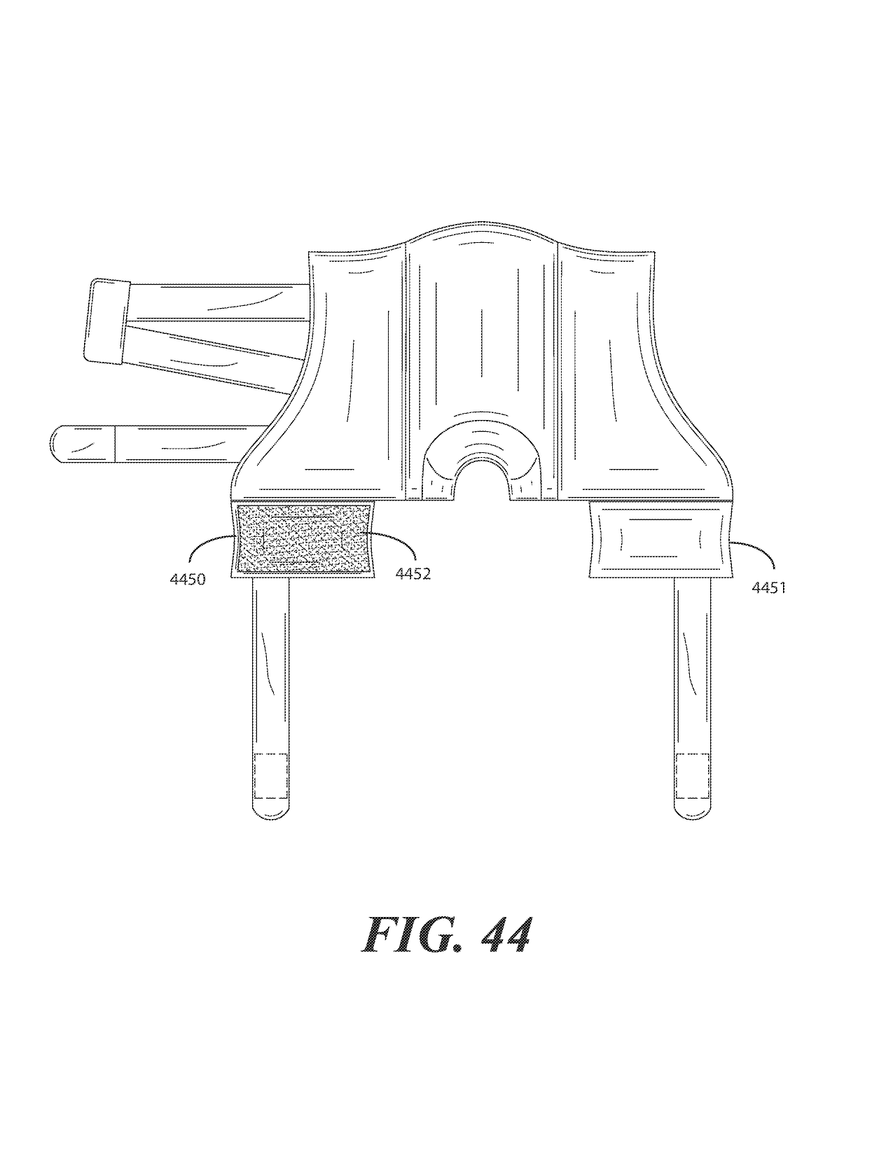

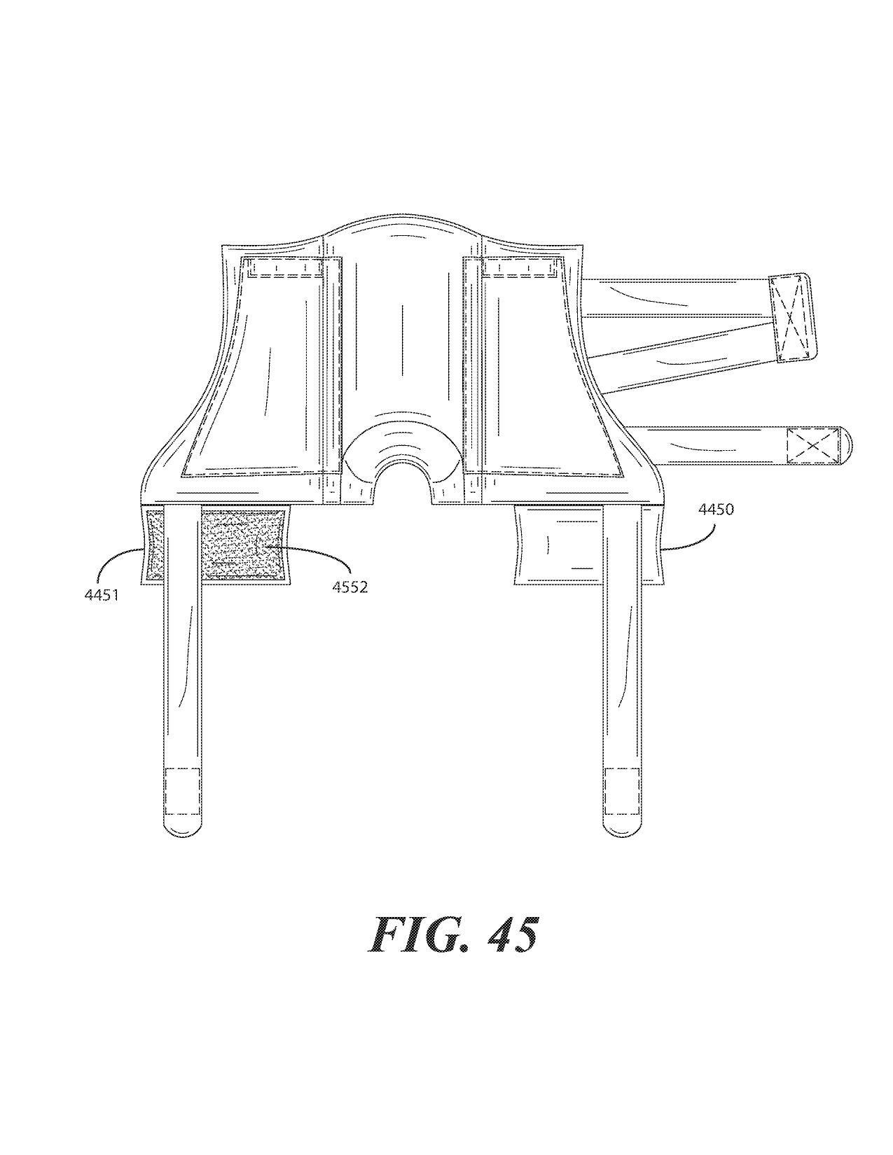

In FIGS. 7-14, a single foot engaging section 704 is shown. However, in other embodiments, one example of which is shown in FIGS. 44-45, the foot engaging section is divided into a first foot engaging flap 4450 and a second foot engaging flap 4451 that overlap to define a single foot engaging section. In the embodiment of FIGS. 44-45, a first hook and fastener layer 4452 and second hook and fastener layer 4552 allow the first foot engaging flap 4450 and a second foot engaging flap 4451 to couple together.

In one or more embodiments, the heel protector comprises a first foot engaging flap and a second foot engaging flap that overlap to define a single foot engaging section. This configuration, as illustrated in FIGS. 44-45, includes a first hook and fastener layer and a second hook and fastener layer that allow the first foot engaging flap and the second foot engaging flap to couple together. This overlapping design offers several benefits over a single foot engaging section.

First, the overlapping foot engaging flaps provide enhanced adjustability. The first hook and fastener layer and the second hook and fastener layer enable the user to adjust the tightness and fit of the foot engaging section more precisely. This adjustability ensures a secure and comfortable fit for various foot sizes and shapes, accommodating individual patient needs and preferences. The ability to fine-tune the fit can also help to alleviate pressure points and reduce the risk of discomfort or injury.

Second, the overlapping design enhances the stability and support provided by the heel protector. By coupling the first foot engaging flap and the second foot engaging flap together, the overlapping configuration creates a more robust and secure attachment. This increased stability helps to prevent the foot engaging section from shifting or loosening during use, maintaining proper alignment and positioning of the heel protector. The secure fit provided by the overlapping flaps ensures that the heel protector remains effective in providing support and protection to the foot and leg.

Additionally, the overlapping foot engaging flaps offer improved ease of use. The first hook and fastener layer and the second hook and fastener layer allow for quick and straightforward attachment and detachment of the foot engaging section. This ease of use is beneficial in medical settings where time and efficiency are important. Healthcare providers can easily apply and adjust the heel protector, ensuring that the patient receives the necessary support and protection without unnecessary delays or complications.

Overall, the use of a first foot engaging flap and a second foot engaging flap that overlap to define a single foot engaging section, coupled with the first hook and fastener layer and the second hook and fastener layer, provides significant advantages. These include enhanced adjustability, increased stability and support, and improved ease of use. These benefits contribute to the overall effectiveness and user experience of the heel protector, ensuring optimal comfort, protection, and functionality for the wearer.

Turning now back to FIGS. 7-14, the foot engaging section 704 is shown as being selectively detached from the first side leg engaging section 701. In one or more embodiments, foot engaging section 704 is fixedly attached to one side leg engaging section, namely, the second side leg engaging section 702, and can be selectively attached to the other side leg engaging section, namely, the first side leg engaging section 701. This selective “attachability” allows for easy and secure attachment and detachment of the foot engaging section 704.

Additionally, in FIGS. 7-14 the first side leg engaging section tab 714 and the second side leg engaging section tab 715 can be observed. In one or more embodiments, the first side leg engaging section tab 714 and the second side leg engaging section tab 715 extend from the foot engaging section 704 and are used for attachment to the side leg engaging sections. In this view, they are shown detached from the first side leg engaging section.

In one or more embodiments, the first side leg engaging section tab 714 and the second side leg engaging section tab 715 are selectively attachable to one of the first side leg engaging section 701 or the second side leg engaging section 702 and are fixedly attached the foot engaging section 704. In the illustrative embodiment of FIGS. 7-14, the first side leg engaging section tab 714 and the second side leg engaging section tab 715 are selectively attachable to the first side leg engaging section 701. However, in other embodiments, such as where the foot engaging section 704 is fixedly coupled to the first side leg engaging section 701, the first side leg engaging section tab 714 and the second side leg engaging section tab 715 will be selectively attachable to the second side leg engaging section 702.

Other configurations will be obvious to those of ordinary skill in the art having the benefit of this disclosure. Illustrating by example, if the foot engaging section 704 is fixedly attached to the third side leg engaging section 703, the first side leg engaging section tab 714 and the second side leg engaging section tab 715 could attach to the first side leg engaging section 701 and second side leg engaging section 702, respectively.

In one or more embodiments, the third side leg engaging section 703 of the heel protector 700 includes a unique feature known as the heel receiver 705, which is defined by a peninsular aperture 706. In one or more embodiments, the heel receiver 705 is situated within the third side leg engaging section 703 and serves as a specialized area designed to accommodate the heel of the wearer. In one or more embodiments, the peninsular aperture 706 is centrally located at the edge of the third side leg engaging section 703 and provides a secure and comfortable fit for the heel. This design ensures that the heel is properly supported and protected, enhancing the overall effectiveness of the heel protector 700 in providing therapeutic benefits and preventing complications during therapy. This design also relieves pressure from the heel when the heel protector 700 is applied to a patient’s leg.

In one or more embodiments, the attachment of the foot engaging section 704 to the second side leg engaging section 702 is done using stitching to fixedly attach a first side leg engaging section tab 716 and a second side leg engaging section tab 717. This method involves sewing the first side leg engaging section tab 716 and the second side leg engaging section tab 717 extending distally from foot engaging section 704 to the second side leg engaging sections 702, creating a secure and durable connection. However, in alternate embodiments, there are several alternatives to stitching that can be utilized for this attachment.

Illustrating by example, in another embodiment the use of adhesive bonding replaces the stitching, with a strong adhesive is applied to join the first side leg engaging section tab 716 and the second side leg engaging section tab 717 extending distally from foot engaging section 704 and the side leg engaging sections. Another option is the use of fasteners such as snaps or buttons, which allow for easy attachment and detachment of the first side leg engaging section tab 716 and the second side leg engaging section tab 717 extending distally from foot engaging section 704 and the side leg engaging sections. Additionally, hook and loop fasteners, commonly known as Velcro, can be employed to provide a secure yet adjustable attachment. These alternatives offer flexibility in the attachment method, allowing for customization based on the specific requirements and preferences of the user.

In the embodiment illustrated in FIGS. 46-47, the foot engaging section 4604 is fully detached from the side leg engaging section 4602, differing from other embodiments where the foot engaging section is fixedly attached to one of the side leg engaging sections. Instead, the foot engaging section 4604 is held in place by the first side leg engaging strap 4612 and the second side leg engaging strap 4704. This design allows for greater flexibility and adjustability in securing the foot engaging section to the leg engaging section.

The detachment of the foot engaging section 4604 from the side leg engaging section 4602 provides several benefits. First, the design allows for easier application and removal of the heel protector, as the foot engaging section can be independently adjusted without the need to manipulate the entire leg engaging section. This can be particularly advantageous in medical settings where quick and efficient application and removal are required. Second, the use of the first side leg engaging strap 4612 and the second side leg engaging strap 4704 to secure the foot engaging section 4604 provides a more customizable fit. The straps can be adjusted to accommodate different foot sizes and shapes, ensuring a secure and comfortable fit for the wearer.

Additionally, the fully detached foot engaging section 4604 enhances the overall versatility of the heel protector. The independent adjustment of the foot engaging section allows for targeted support and protection of the foot, which can be beneficial for patients with specific therapeutic needs. The design also facilitates better ventilation and airflow around the foot, reducing the risk of moisture buildup and skin irritation. Overall, the fully detached foot engaging section 4604, secured by the first side leg engaging strap 4612 and the second side leg engaging strap 4704, offers significant advantages in terms of ease of use, adjustability, and comfort.

In some embodiments, the side leg engaging strap 4704 can be fully detached from the side leg engaging section 4602. This configuration allows for greater flexibility and adjustability in securing the foot engaging section 4604 to the leg engaging section. The detachment of the side leg engaging strap 4704 provides several benefits, including easier application and removal of the heel protector, as well as a more customizable fit. The independent adjustment of the side leg engaging strap 4704 ensures that the heel protector can accommodate different foot sizes and shapes, providing a secure and comfortable fit for the wearer.

However, the distal end of the side leg engaging strap 4704 can optionally be sewn to the side leg engaging section 4602 to prevent the foot engaging section 4604 from being separated from the upper assembly of the heel protector. This optional sewing provides an additional layer of security, ensuring that the foot engaging section 4604 remains attached to the side leg engaging section 4602 even during movement or activity. The sewn attachment helps to maintain the integrity and stability of the heel protector, reducing the risk of accidental detachment or misalignment. This configuration offers a balance between flexibility and security, allowing for easy adjustments while ensuring that the foot engaging section 4604 remains securely connected to the upper assembly of the heel protector.

In one or more embodiments, a hook fastener 4611 can be positioned between an attachment point of the side leg engaging strap 4704 and the permanent attachment of the side leg engaging strap 4704 to the second side leg engaging section 4602. This configuration allows the foot engaging section 4604 to be situated in different positions relative to the second side leg engaging section 4602. The hook fastener 4611 provides a secure and adjustable connection, enabling the foot engaging section 4604 to be repositioned as needed to accommodate various foot sizes and shapes, ensuring a comfortable and customized fit for the wearer.

The placement of the hook fastener 4611 between the attachment point and the permanent attachment of the side leg engaging strap 4704 offers several benefits. First, the hook fastener 4611 allows for easy adjustment of the foot engaging section 4604 without the need to detach the entire side leg engaging strap 4704. This enhances the convenience and efficiency of applying and adjusting the heel protector, particularly in medical settings where quick and precise adjustments are often required. Second, the adjustable nature of the hook fastener 4611 ensures that the foot engaging section 4604 can be securely fastened in multiple positions, providing optimal support and stability for the foot and leg.

Moreover, the use of the hook fastener 4611 in this configuration allows for greater flexibility in the design and application of the heel protector. The ability to reposition the foot engaging section 4604 as needed ensures that the heel protector can accommodate a wide range of therapeutic and comfort requirements. This versatility makes the heel protector suitable for various applications, including medical rehabilitation, sports recovery, and everyday wear. The hook fastener 4611 thus plays a significant role in enhancing the overall functionality and adaptability of the heel protector, ensuring that the heel protector meets the diverse needs of the users.

Turning now back to FIGS. 7-14, in one or more embodiments the heel protector 700 features a side leg engaging section strap 712 that extends distally from the foot engaging section 704. In one or more embodiments, the side leg engaging section strap 712 defines a side engagement extension 726 that is coupled to, and extends distally from, a second side 725 of the foot engaging section 704 that is situated opposite a first side 724 of the foot engaging section 704 and is separated by a width of the foot engaging section 704. In one or more embodiments, this side leg engaging section strap 712 serves an important role in providing additional support and stability to the heel protector 700 when it attaches to a second loop fastener panel 805 when the foot engaging section 704 is attached to the first side leg engaging section 701.

In one or more embodiments, unlike the first side leg engaging section tab 714 and the second side leg engaging section tab 715, which extend distally from the foot engaging section 704 at an orthogonal angle relative to the edge of the foot engaging section 704 to which they are coupled, in one or more embodiments the side leg engaging section strap 712 extends at a different angle and different distance from the foot engaging section 704. This design allows a hook fastener 713 attached to the side leg engaging section strap 712, one example of which is a hook fastener, to securely attach to loop fastener panels, one example of which is defined by first loop fastener panel 801 attached to the first side leg engaging section 701, on the exterior surfaces of the first side leg engaging section 701 and the second side leg engaging section 702 when the foot engaging section 704 is selectively attached to the first side leg engaging section 701.

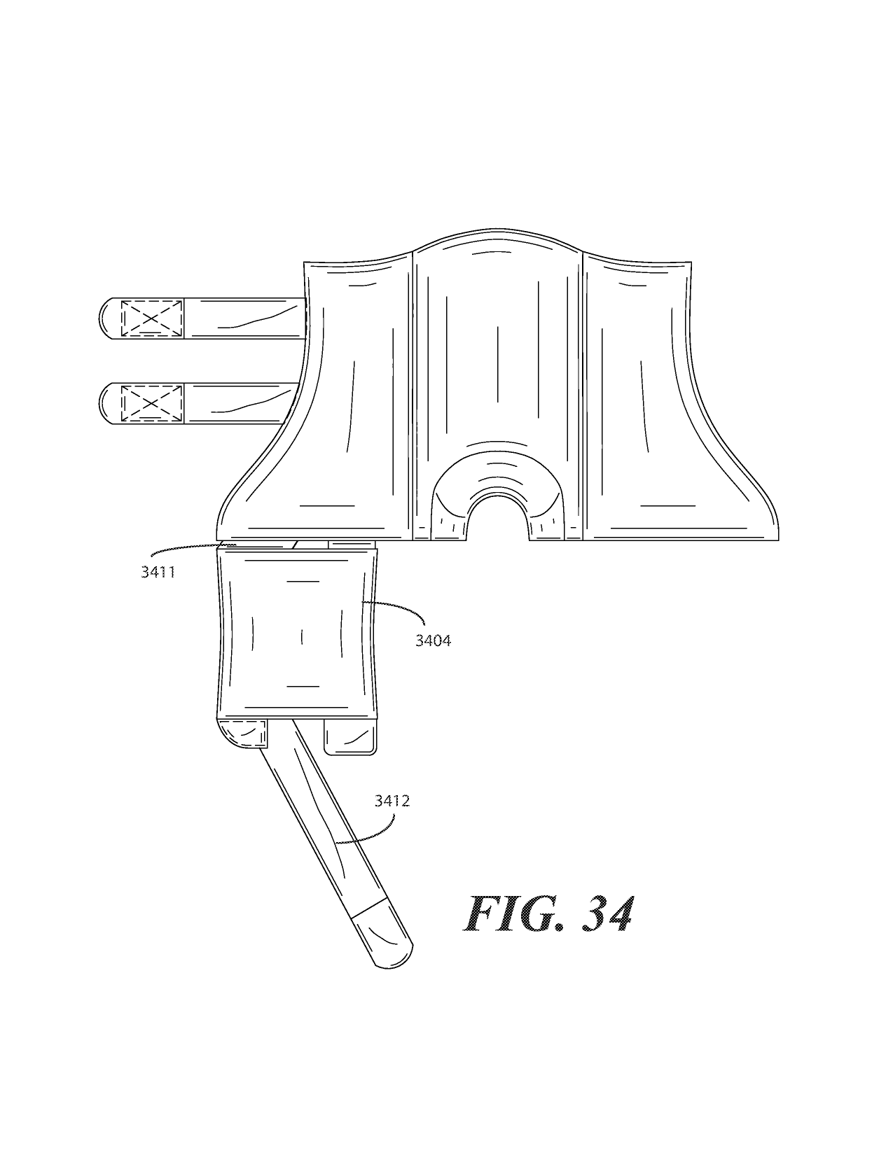

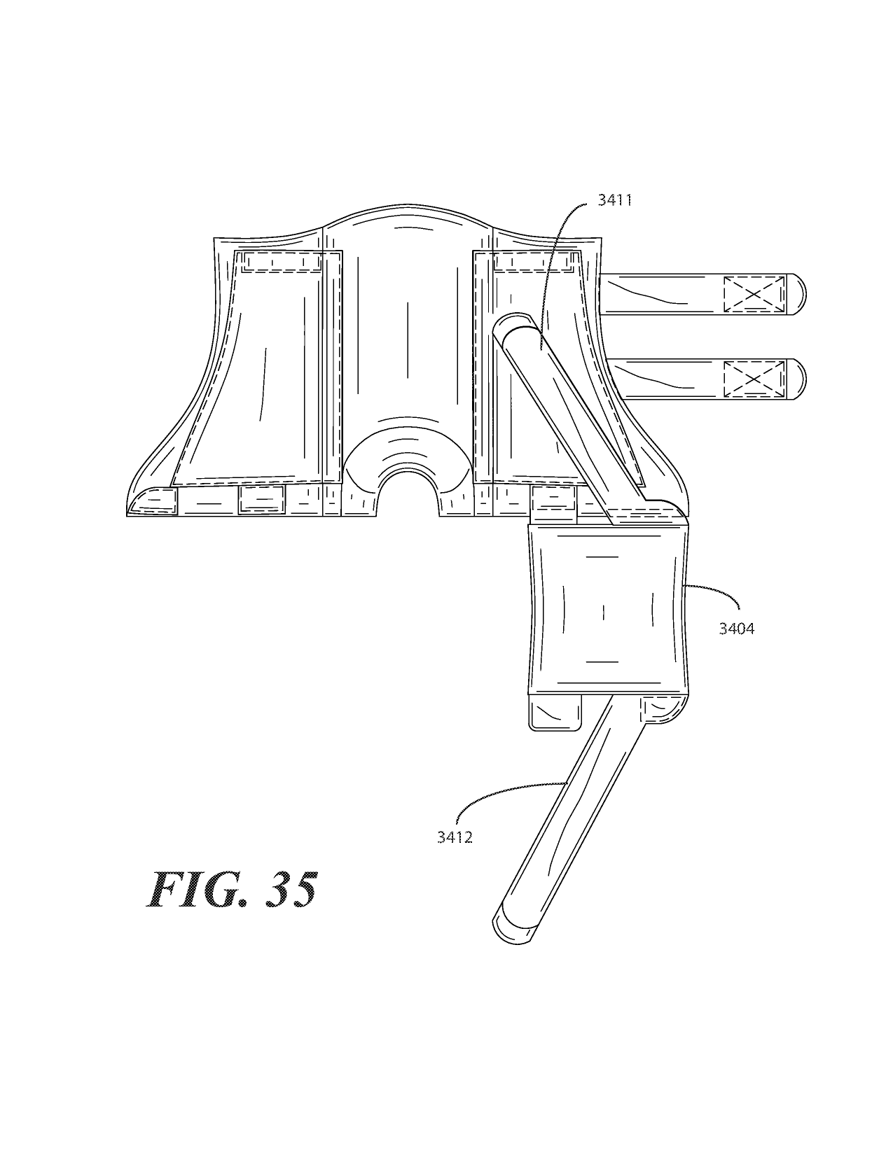

In other embodiments, such as that shown in FIGS. 34-35, the first side leg engaging section tab 3411 and the second side leg engaging section tab 3412 have a hockey stick shape, with a base of the hockey stick intersecting the foot engaging section 3404. This specific shape configuration offers several benefits.

The hockey stick shape provides a larger surface area for attachment, enhancing the stability and security of the connection between the foot engaging section 3404 and the side leg engaging sections. The increased surface area allows for a more robust and durable attachment, reducing the risk of detachment or failure during use.

Additionally, the hockey stick shape facilitates a more ergonomic fit around the leg. The curved design of the hockey stick shape conforms to the natural contours of the leg, providing a comfortable and secure fit. This ergonomic fit helps to distribute pressure evenly across the leg, reducing the risk of pressure points and discomfort. The shape also allows for better flexibility and movement, ensuring that the heel protector can accommodate various leg sizes and shapes while maintaining a secure attachment.

Moreover, the hockey stick shape enhances the overall aesthetic appeal of the heel protector. The visually interesting design adds a touch of sophistication and style to the heel protector, making the heel protector more appealing to users. The shape also contributes to a streamlined and cohesive appearance, enhancing the overall visual composition of the heel protector. This combination of functionality and aesthetics ensures that the heel protector not only provides effective support and protection but also offers a visually pleasing design.

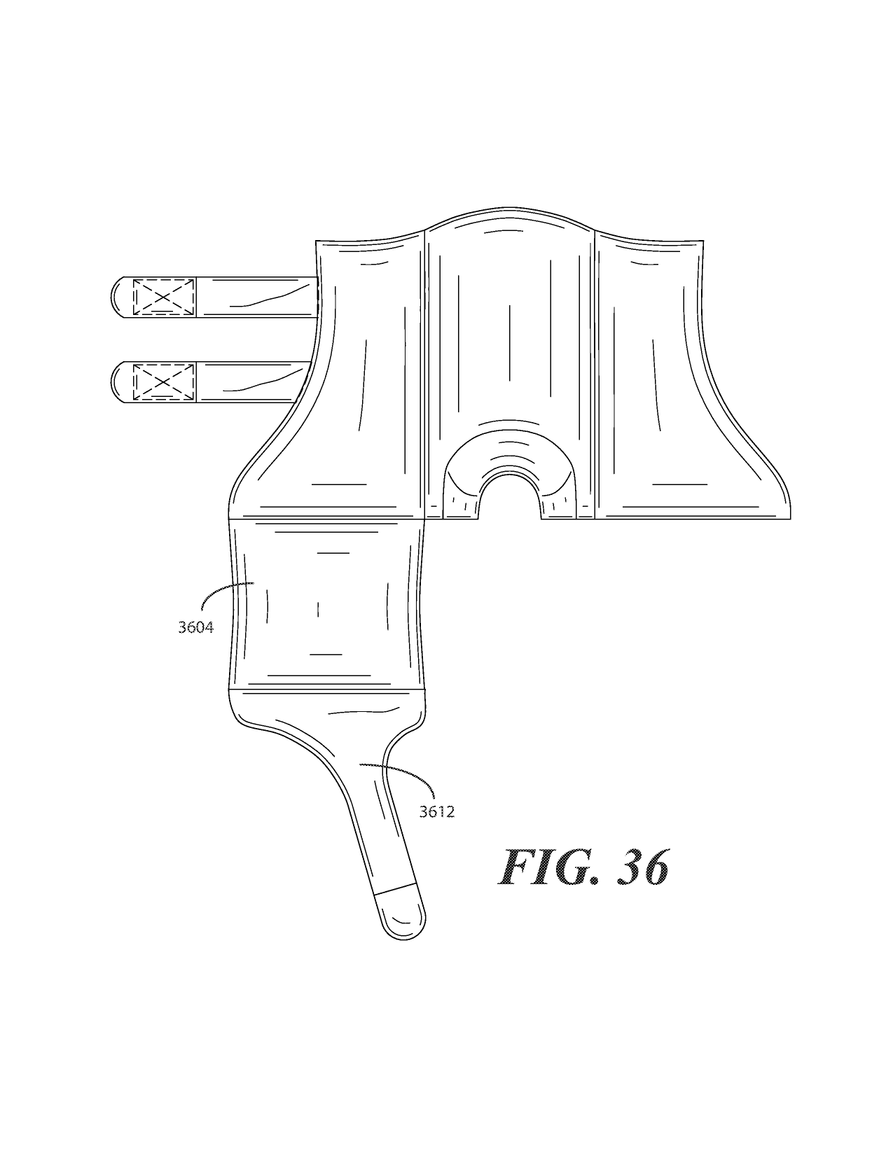

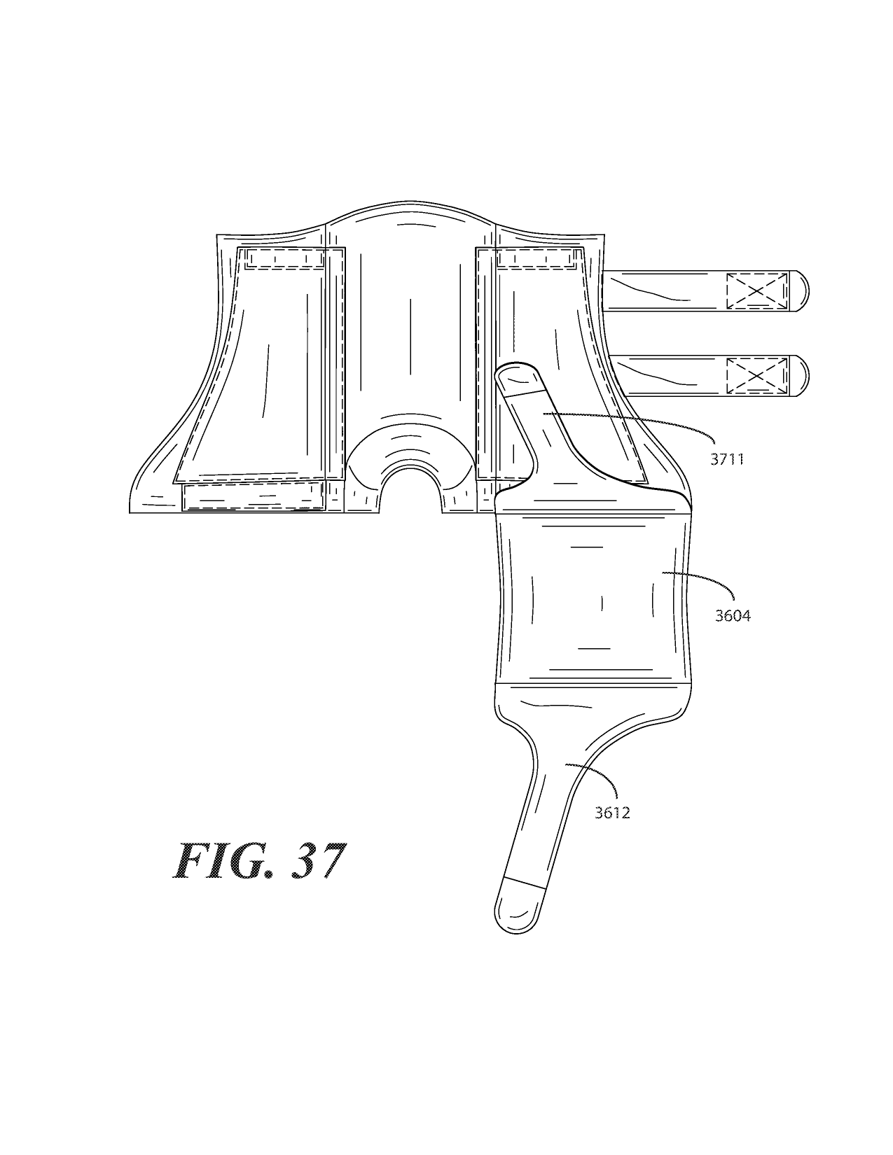

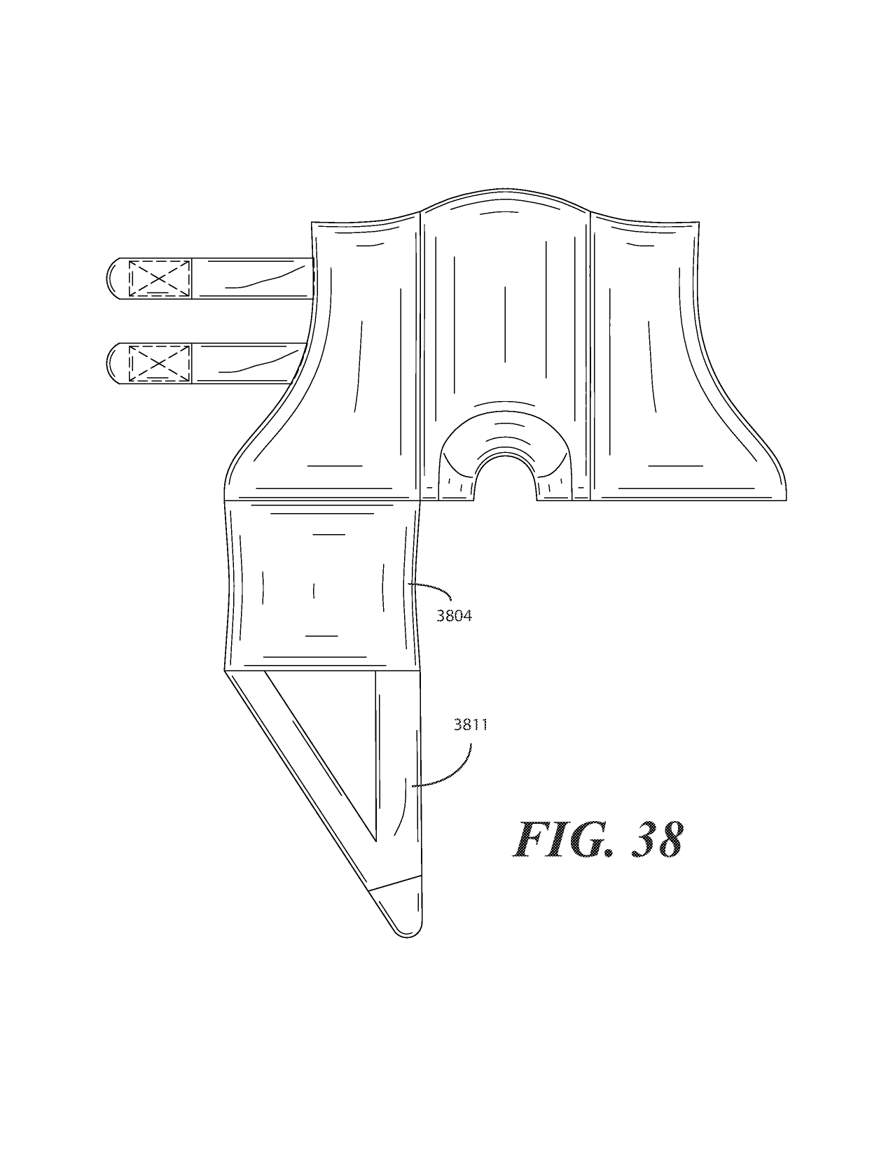

As shown in FIGS. 36-37, in other embodiments the first side leg engaging section tab 3711 and the second side leg engaging section tab 3612 have a slanted partial paint brush shape, with the portion of the paint brush entering the can at a slant intersecting the foot engaging section 3604. This specific shape configuration offers several benefits.

The slanted partial paint brush shape provides a larger surface area for attachment, enhancing the stability and security of the connection between the foot engaging section 3604 and the side leg engaging sections. The increased surface area allows for a more robust and durable attachment, reducing the risk of detachment or failure during use.

Additionally, the slanted partial paint brush shape facilitates a more ergonomic fit around the leg. The slanted design of the paint brush shape conforms to the natural contours of the leg, providing a comfortable and secure fit. This ergonomic fit helps to distribute pressure evenly across the leg, reducing the risk of pressure points and discomfort. The shape also allows for better flexibility and movement, ensuring that the heel protector can accommodate various leg sizes and shapes while maintaining a secure attachment.

Moreover, the slanted partial paint brush shape enhances the overall aesthetic appeal of the heel protector. The visually interesting design adds a touch of sophistication and style to the heel protector, making the heel protector more appealing to users. The shape also contributes to a streamlined and cohesive appearance, enhancing the overall visual composition of the heel protector. This combination of functionality and aesthetics ensures that the heel protector not only provides effective support and protection but also offers a visually pleasing design.

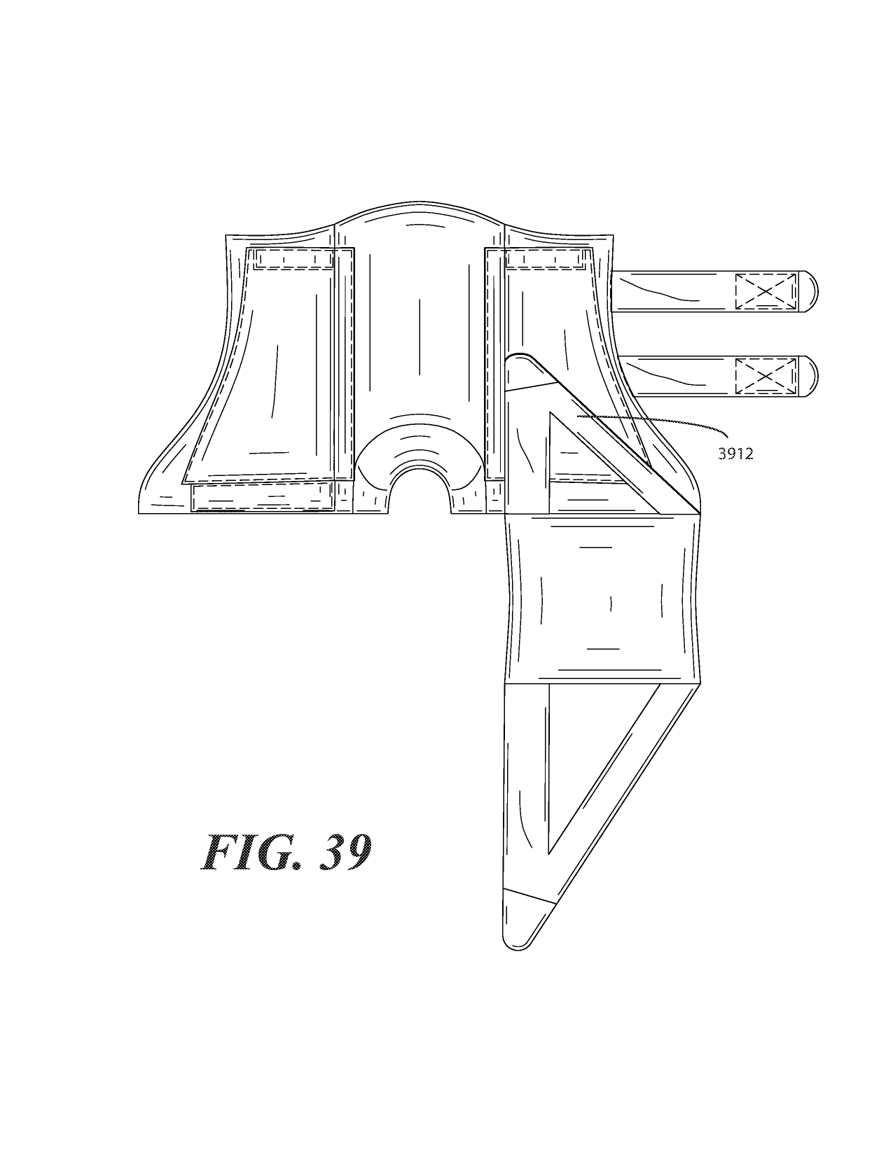

In other embodiments, such as that shown in FIGS. 38-39, the first side leg engaging section tab 3811 and the second side leg engaging section tab 3912 define two sides of a triangle, with the foot engaging section 3804 defining a base of the triangle. This triangular configuration offers several benefits.

The triangular shape provides a larger surface area for attachment, enhancing the stability and security of the connection between the foot engaging section 3804 and the side leg engaging sections. The increased surface area allows for a more robust and durable attachment, reducing the risk of detachment or failure during use.

Additionally, the triangular shape facilitates a more ergonomic fit around the leg. The angled design of the triangular shape conforms to the natural contours of the leg, providing a comfortable and secure fit. This ergonomic fit helps to distribute pressure evenly across the leg, reducing the risk of pressure points and discomfort. The shape also allows for better flexibility and movement, ensuring that the heel protector can accommodate various leg sizes and shapes while maintaining a secure attachment.

Moreover, the triangular shape enhances the overall aesthetic appeal of the heel protector. The visually interesting design adds a touch of sophistication and style to the heel protector, making the heel protector more appealing to users. The shape also contributes to a streamlined and cohesive appearance, enhancing the overall visual composition of the heel protector. This combination of functionality and aesthetics ensures that the heel protector not only provides effective support and protection but also offers a visually pleasing design.

Turning now back to FIGS. 7-14, in one or more embodiments a second side leg engaging section strap 804 having a second fastener 711 attached thereto can attach to a second loop fastener panel 805 in a similar manner. The side leg engaging section straps 712,804 enhance the overall functionality of the heel protector 700, ensuring a snug and comfortable fit while providing the necessary support for the leg and foot.

In one or more embodiments, the leg engaging section defined by the first side leg engaging section 701, the second side leg engaging section 702, and the third side leg engaging section 703 of the heel protector 700 is designed to provide not only support and protection but also a comfortable and cushioned experience for the wearer. In one or more embodiments, the leg engaging section defined by the first side leg engaging section 701, the second side leg engaging section 702, and the third side leg engaging section 703 of the heel protector 700 comprises a compressible cushion layer that can be made from various materials. In one embodiment, the cushion layer can be composed of an organic batting, such as cotton or wool, which offers softness and breathability.

Alternatively, an inorganic batting material, such as foam or synthetic fibers, can be used to provide additional cushioning and support. Combinations of these materials can also be employed to achieve the desired balance of comfort and functionality. Other potential batting material suggestions include memory foam, gel-infused foam, or even specialized medical-grade cushioning materials. The choice of batting material can be tailored to meet specific requirements, such as moisture-wicking properties, antimicrobial features, or temperature regulation, ensuring optimal comfort and performance for the wearer. Other materials will be obvious to those of ordinary skill in the art having the benefit of this disclosure.

In one or more embodiments, the first loop fastener panel 801 and the second loop fastener panel 805 are attached to the first side leg engaging section 701 and the second side leg engaging section 702, respectively, using stitching. This method involves sewing the loop fastener panels 801,805 securely onto the exterior surfaces of the side leg engaging sections 701,702, ensuring a strong and durable attachment. However, it should be noted that there can be alternative methods to stitching that can be used for attaching the loop fastener panels 801,805.

One alternative is the use of adhesive bonding, where a strong adhesive is applied to adhere the loop fastener panels 801,805 to the side leg engaging sections 701,702. Another option is the use of heat sealing, where the loop fastener panels 801,805 are thermally bonded to the side leg engaging sections 701,702. Additionally, mechanical fasteners such as rivets or snaps can be utilized to provide a secure attachment. These alternatives offer flexibility in the attachment method, allowing for customization based on the specific requirements and preferences of the heel protector design.

In one or more embodiments, the first loop fastener panel 801 and the second loop fastener panel 805 of the heel protector 700 can be designed to mirror the shape of the first side leg engaging section 701 and the second side leg engaging section 702, respectively. This mirroring shape ensures a pleasant aesthetic when the first loop fastener panel 801 and the second loop fastener panel 805 are attached to the exterior surfaces of the side leg engaging sections 701,702.

However, it will be obvious to those of ordinary skill in the art having the benefit of this disclosure that the first loop fastener panel 801 and the second loop fastener panel 805 can also take other shapes depending on the specific design requirements and preferences. For example, the first loop fastener panel 801 and the second loop fastener panel 805 can be rectangular, oval, or even custom-shaped to accommodate unique design elements or enhance the overall aesthetics of the heel protector 700. The choice of shape for the first loop fastener panel 801 and the second loop fastener panel 805 can be tailored to meet functional and design considerations, providing versatility and customization options for the attachment mechanism.

In one or more embodiments the heel protector 700 comprises at least one side leg engaging section tab 714,715 coupled to the foot engaging section 704 that is shorter than the side leg engaging section strap 712 and is selectively attachable to either the first side leg engaging section 701 or the second side leg engaging section 702. This configuration allows for additional flexibility in the attachment mechanism, as the shorter tab can be easily secured to the side leg engaging sections using hook fasteners.

In one or more embodiments, a hook fastener is coupled to the at least one side leg engaging section tab 714,715. This hook fastener enhances the attachment strength and stability by securely fastening the tab to the loop fastener sections 802,803 on the first side leg engaging section 701. This ensures a reliable connection between the foot engaging section 704 and the first side leg engaging section 701.

Some examples of substitutes for hook and loop fasteners include snap buttons, buckles, magnetic closures, adhesive strips, zipper closures, or even adjustable straps with clasps. These alternative fastening mechanisms offer flexibility in design and can be chosen based on factors such as ease of use, durability, adjustability, and aesthetic appeal. Still other substitutes will be obvious to those of ordinary skill in the art having the benefit of this disclosure.

In one or more embodiments, the at least one side leg engaging section tab comprises a first side leg engaging section tab 714 and a second side leg engaging section tab 715 that extend distally from the foot engaging section 704. In one or more embodiments, the first side leg engaging section tab 714 originates from a first corner section 718 of the foot engaging section 704, while the second side leg engaging section tab 715 extends distally from a second corner section 719 of the foot engaging section 704. This arrangement allows for a balanced and secure attachment of the foot engaging section 704 to the first side leg engaging section 701.

In the illustrative embodiment of FIGS. 7-14, it is highlighted that the first side leg engaging section tab 714 and the second side leg engaging section tab 715 extend distally at different distances from the foot engaging section 704. This design variation ensures a proper fit and alignment of the tabs with the corresponding side leg engaging sections, accommodating any anatomical differences or specific requirements of the wearer. Moreover, in this illustrative embodiment the second side leg engaging section tab 715 has at least one rounded corner, while the first side leg engaging section tab 714 is square. This rounded corner feature enhances comfort and reduces the risk of irritation or discomfort caused by sharp edges, ensuring a pleasant wearing experience for the user. Other shapes for the first side leg engaging section tab 714 and the second side leg engaging section tab 715 will be obvious to those of ordinary skill in the art having the benefit of this disclosure.

In one or more embodiments, the side leg engaging section tabs 716,717 fixedly coupling the foot engaging section 704 to the second side leg engaging section 702, together with and edge defined by the first side 724 of the foot engaging section 704 and an edge of the second side leg engaging section 702, define a rectangular aperture 720. This rectangular aperture 720 allows for easy folding of the foot engaging section 704 relative to the second side leg engaging section 702, thereby ensuring proper alignment and fit of the leg within the heel protector 700.

In one or more embodiments, a pair of leg insertion aperture spanning straps 707,708 are fixedly coupled to, and extend distally from, the second side leg engaging section 702. In one or more embodiments these straps extend from the second side leg engaging section 702 and are designed to span across the leg insertion aperture (1501), shown below in FIGS. 15-22. The purpose of these straps is to provide additional support and stability to the heel protector 700, ensuring a secure fit and preventing any unwanted movement or displacement.

In the embodiment illustrated in FIGS. 7-14, the two leg insertion aperture spanning straps 707,708 extend from the second side leg engaging section 702 parallel to each other, each terminating in separate hook fasteners 709,710. This parallel configuration allows for a straightforward and balanced attachment to the first loop fastener panel 801, ensuring a secure fit around the leg insertion aperture 1501. The parallel arrangement of the straps provides even distribution of tension across the leg insertion aperture, contributing to the overall stability and support of the heel protector 700.

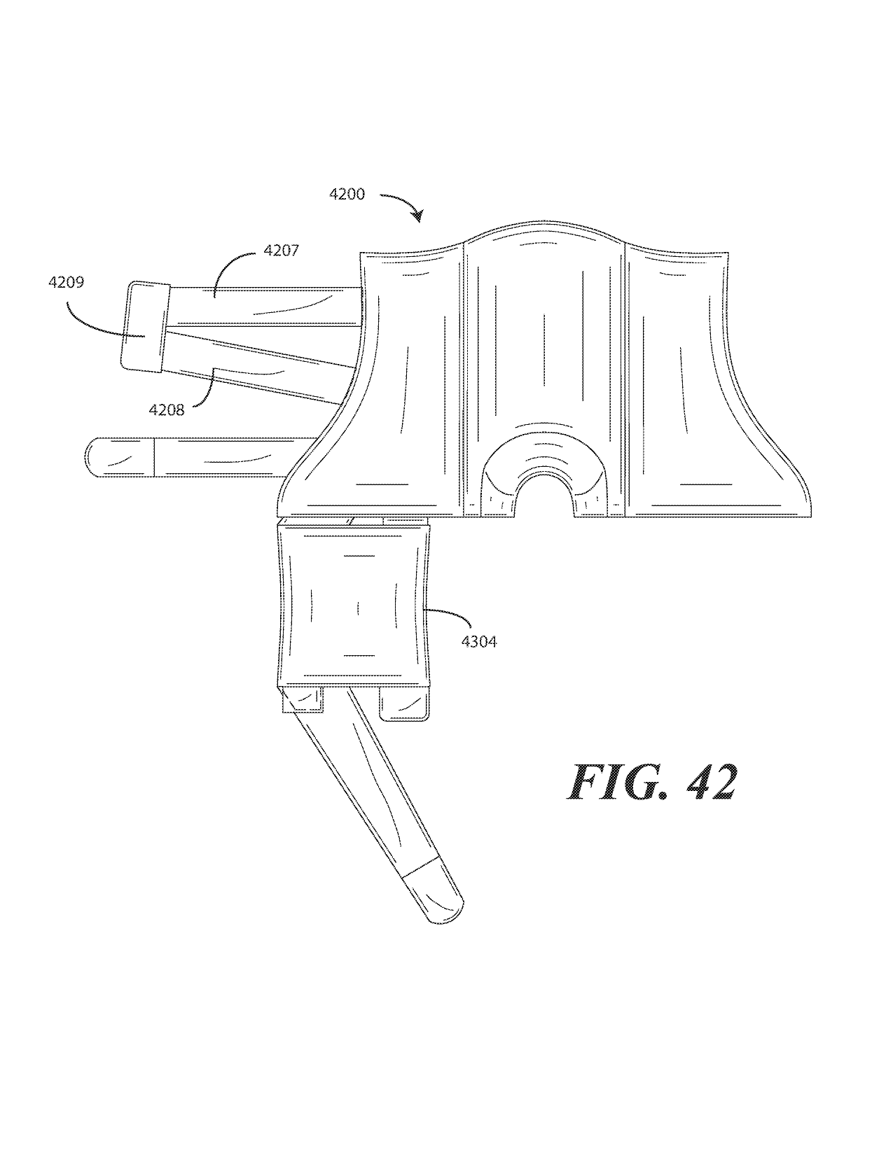

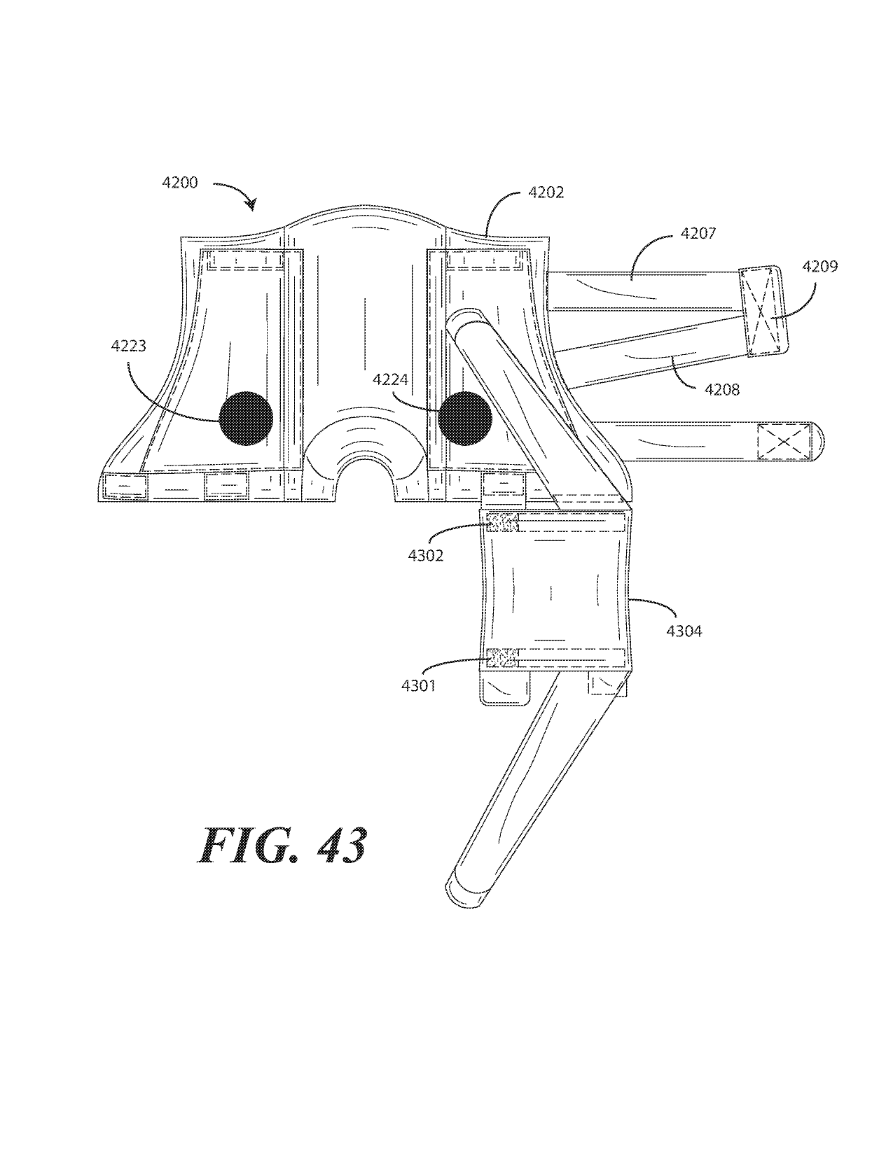

By contrast, in the embodiment depicted in FIGS. 42-43, the two leg insertion aperture spanning straps 4207,4208 extend distally from the second side leg engaging section 4202 toward each other, converging to a single hook fastener 4209. This converging configuration offers several potential benefits. First, the single hook fastener 4209 simplifies the attachment process, as only one fastener needs to be secured to the first loop fastener panel 801. This can enhance ease of use, particularly for patients or healthcare providers who may have limited dexterity or require quick application and removal of the heel protector 4200.

Additionally, the converging straps 4207,4208 create a more centralized point of attachment, which can improve the overall fit and alignment of the heel protector 4200. By directing the tension toward a single hook fastener 4209, the design ensures that the straps pull evenly from both sides, reducing the risk of uneven pressure distribution or misalignment. This can enhance the comfort and effectiveness of the heel protector 4200, providing a more secure and supportive fit for the wearer.

Moreover, the converging configuration of the straps 4207,4208 can contribute to a more streamlined and aesthetically pleasing design. The single hook fastener 4209 reduces the visual clutter associated with multiple fasteners, creating a cleaner and more cohesive appearance. This can be particularly beneficial for patients who value both functionality and aesthetics in their medical devices. Overall, the converging straps with a single hook fastener offer a practical and visually appealing alternative to the parallel strap configuration, enhancing the user experience and effectiveness of the heel protector 4200.

In the embodiment shown in FIGS. 42-43, the heel protector includes optional air holes 4223,4224 that allow ventilation to the ankle region of the leg. These air holes are strategically placed to enhance airflow and reduce moisture buildup, thereby promoting a more comfortable and hygienic environment for the wearer. The air holes 4223,4224 are positioned along the side leg engaging sections, ensuring that the ankle region receives adequate ventilation without compromising the structural integrity or support provided by the heel protector.

In one or more embodiments, the air holes 4223,4224, while shown as being circular, could take other shapes, including squares, triangles, or spline closed shapes. The choice of shape for the air holes can be tailored to meet specific design requirements and preferences, providing flexibility in the overall configuration of the heel protector. For example, square air holes can offer a modern and geometric aesthetic, while triangular air holes can provide a distinct and visually interesting design element. Spline closed shapes, which are defined by smooth, continuous curves, can create an organic and fluid appearance, enhancing the overall visual appeal of the heel protector.

Additionally, the different shapes of the air holes can influence the airflow and ventilation properties of the heel protector. Square and triangular air holes may provide more structured and directional airflow, while spline closed shapes can allow for a more diffused and even distribution of air. This customization of air hole shapes ensures that the heel protector can be optimized for various therapeutic and comfort requirements, promoting better ventilation and reducing the risk of moisture buildup and skin irritation.

Moreover, the use of different air hole shapes can contribute to the structural integrity and durability of the heel protector. The specific geometry of the air holes can be designed to minimize stress concentrations and enhance the overall strength of the material. This ensures that the heel protector remains robust and reliable, even with the inclusion of multiple air holes for ventilation purposes. The ability to customize the shape of the air holes provides a versatile and adaptable solution, ensuring that the heel protector meets the diverse needs of users while maintaining effectiveness and durability.

It should be noted that any of the embodiments described in the instant disclosure can also include such air holes. The placement and number of air holes can be customized based on specific requirements and preferences. For example, air holes can be located at various positions along the side leg engaging sections to optimize ventilation and comfort. This flexibility allows for the adaptation of the heel protector to different user needs and environmental conditions, ensuring that the heel protector remains effective and comfortable in a wide range of applications. Other variations will be obvious to those of ordinary skill in the art having the benefit of this disclosure.

In one or more embodiments, micro hooks 4301,4302 can be sewn on top of the side leg engaging tabs to secure the foot engaging section 4304 to the second side leg engaging section 4202. The micro hooks 4301,4302 are designed to provide a secure and reliable attachment mechanism, ensuring that the foot engaging section 4304 remains firmly in place during use. The micro hooks 4301,4302 can be strategically positioned on the side leg engaging tabs to maximize their effectiveness. Additionally, placement of the micro hooks 4301,4302 can be strategic so as to allow a health care services provider to perform skin checks on a patient wearing the heel protector 4200.

The sewing process involves attaching the micro hooks 4301,4302 to the foot engaging section 4304 using a durable and high-strength thread. This ensures that the micro hooks 4301,4302 are securely fastened to the tabs, preventing any detachment or loosening during movement or activity. The placement of the micro hooks 4301,4302 on the side leg engaging tabs allows for easy and quick attachment to the corresponding loop fastener panels on the second side leg engaging section 4202, providing a convenient and efficient fastening solution.

In one or more embodiments, the micro hooks 4301,4302 are arranged in a pattern that optimizes their gripping strength and distribution of tension. This pattern can be customized based on the specific design requirements and preferences, ensuring that the attachment mechanism provides a secure and comfortable fit for the wearer. The use of micro hooks 4301,4302 enhances the overall functionality and performance of the heel protector, offering a reliable and adjustable fastening solution that can accommodate various foot sizes and shapes.

Turning now back to FIGS. 7-14, in one or more embodiments leg insertion aperture spanning straps 707,708 include hook fasteners 709,710 that are selectively attachable to the first loop fastener panel 801. This attachment mechanism allows for easy and adjustable fastening of the straps to the loop fastener panel 801 on the first side leg engaging section 701. By utilizing hook fasteners 709,710, the straps can be securely attached and detached as needed, providing flexibility and convenience in adjusting the fit and tension of the heel protector 700.

The inclusion of these leg insertion aperture spanning straps 707,708 with hook fasteners 709,710 enhances the overall functionality and effectiveness of the heel protector 700. They contribute to the stability and support of the device, ensuring a snug and comfortable fit for the wearer. Additionally, the selective attachment feature allows for customization and adjustment based on individual preferences and requirements.

Turning now to FIGS. 15-22, illustrated therein is the heel protector 700 in its assembled form, also known as a “wearable configuration.” FIGS. 15 and 16 illustrate perspective views of the heel protector 700 with an attachment mechanism of the foot engaging section 704 shown attached to the first side leg engaging section 701 in accordance with one or more embodiments of the disclosure, while FIG. 17 illustrates a top plan view thereof. FIG. 18 illustrates a bottom plan view of the heel protector 700, while FIG. 19 illustrates a right elevation view.

FIG. 20 illustrates a left elevation view of the heel protector 700, while FIG. 21 illustrates a front elevation view of the heel protector 700. FIG. 22 illustrates a rear elevation view of the heel protector. It will be appreciated by those of ordinary skill in the art having the benefit of this disclosure that the pertinent aspects of the heel protector 700 can be easily seen among the views of FIGS. 15-22, as these figures provide a comprehensive visual representation of the various configurations and components of the heel protector 700.

From the wearable configuration with the attachment mechanism of the foot engaging section 704 attached to the first side leg engaging section 701, which is in contrast to the substantially planar configuration with the attachment mechanism detached that was shown above in FIGS. 1-14, the figures showcase the versatility and functionality of the heel protector 700. The placement and design of the side leg engaging section straps 712,804, the first side leg engaging section tabs 714,715, and fasteners other can be clearly observed, allowing for a better understanding of how the heel protector 700 provides a secure and comfortable fit. These detailed views provide valuable insights into the overall structure and operation of the heel protector 700, making it easier to appreciate its innovative features and benefits.

As shown in FIGS. 15-22, the first side leg engaging section 701 of the leg engaging section, the second side leg engaging section 702 of the leg engaging section, and the third side leg engaging section 703 of the leg engaging section have been folded such hat the first side leg engaging section 701 and the second side leg engaging section 702 form substantially orthogonal angles relative to the third side leg engaging section 703. Moreover, the first side leg engaging section tab 714 and the second side leg engaging section tab 715 have been coupled to the loop fastener sections 802,803 on the first side leg engaging section 701.

Doing so not only holds the first side leg engaging section 701, the second side leg engaging section 702, and the third side leg engaging section 703 in their orthogonal relationship that defines a leg insertion aperture 1501, but also allows the side leg engaging section straps 712,804 to couple to the first loop fastener panel 801 and the second loop fastener panel 805, respectively. As shown in these figures, since the side leg engaging section straps 712,804 extend distally from the foot engaging section 704 at an angle, this creates the visual appearance of the side leg engaging section straps 712,804 emanating from the heel protector 700 at the toes and passing up the lower leg of the patient toward the calf.

In one or more embodiments, the side leg engaging section straps 712,804 are equipped with loop fasteners on their exteriors. This allows the hook fasteners 709,710 of the leg insertion aperture spanning straps 707,708 to attach not only to the first loop fastener panel 801, but also to the side leg engaging section straps 712,804, as can be seen in FIGS. 16 and 20. This ensures a reliable connection between the foot engaging section 704 and the first side leg engaging section 701. As shown, the leg insertion aperture spanning straps 707,708 extending distally from the second side leg engaging section 702 to pass over the leg insertion aperture 1501 to securely retain the first side leg engaging section 701 and the second side leg engaging section 702 to the sides of the leg.

In one or more embodiments, the heel protector 700 of FIGS. 15-22 offers a unique advantage over prior art by allowing easy access to a patient's foot without the need to remove the entire heel protector 700 from the leg. By simply unfastening the first side leg engaging section tab 714 from loop fastener section 802, the second side leg engaging section tab 715 from loop fastener section 803, and the hook fastener 713 of the side leg engaging section strap 712 from the first loop fastener panel 801, the foot engaging section 704 can be folded away from the patient's foot, providing convenient access for examination or treatment. This feature eliminates the need for complete removal of the heel protector 700, thereby saving time and effort for both the patient and healthcare provider.

This innovative design offers several advantages over prior art that does not allow the foot engaging section 704 to fold away. Illustrating by example, in one or more embodiments it provides a more efficient and streamlined process for checking the patient's foot, as there is no need to remove and reapply the entire heel protector 700. This saves valuable time during medical examinations or procedures, allowing for quicker and more effective patient care.

Additionally, the ability to fold the foot engaging section 704 away from the foot while keeping the rest of the heel protector 700 in place ensures that the leg and heel remain protected and supported throughout the examination or treatment process. This eliminates the risk of accidental injury or discomfort that may arise from complete removal of the heel protector 700.

What’s more, the folding feature of the foot engaging section 704 allows for better visibility and access to the foot, thereby enabling healthcare providers to perform thorough examinations or administer treatments with greater precision. This can lead to more accurate diagnoses and improved patient outcomes.

Overall, the ability to easily check a patient's foot by unfastening the first side leg engaging section tab 714 and the second side leg engaging section tab 715, as well as the side leg engaging section strap 712, and simply folding the foot engaging section 704 away from the first side leg engaging section 701 offers significant advantages over prior art. It enhances efficiency, maintains protection and support for the leg and heel, and improves visibility and access for medical procedures. This innovative design represents a significant advancement in heel protector technology, benefiting both patients and healthcare providers.

In one or more embodiments, the peninsular aperture 706 of the heel receiver 705 in the heel protector 700 allows a patient's heel to be exposed while wearing the heel protector 700. This unique feature provides several advantages that contribute to the overall effectiveness and comfort of the heel protector.

Illustrating by example, in one or more embodiments the exposure of the heel allows for proper ventilation and airflow, which helps to prevent excessive moisture buildup and the development of skin irritations or infections. By allowing air to circulate around the heel, the protector promotes a healthier and more hygienic environment, reducing the risk of discomfort or complications.

Additionally, the exposure of the heel enables healthcare providers to easily monitor and assess the condition of the heel. This is particularly important for patients who may be at risk of developing pressure ulcers or other heel-related issues. With the heel exposed, healthcare providers can closely examine the skin, assess any signs of redness or irritation, and implement appropriate preventive measures or treatments as needed. Overall, heel protectors configured in accordance with embodiments of the disclosure advantageously remove pressure from a patient’s heel while being worn.

Moreover, the exposure of the heel allows for targeted treatments or therapies to be applied directly to the affected area. This is especially beneficial for patients undergoing specific heel-related treatments, such as wound care or the application of topical medications. The ability to access and treat the heel without removing the entire heel protector 700 enhances convenience and efficiency, ensuring that the patient receives the necessary care without unnecessary disruptions.

Overall, the exposure of the heel through the peninsular aperture 706 of the heel receiver 705 in the heel protector 700 offers significant advantages. It promotes ventilation and hygiene, facilitates monitoring and assessment of the heel's condition, and allows for targeted treatments or therapies. These benefits contribute to the overall effectiveness and comfort of the heel protector, enhancing the patient's experience and supporting optimal healing and recovery.

As demonstrated above with reference to FIGS. 1-14, in one or more embodiments the first side leg engaging section tab 714 extends distally less far from the foot engaging section 704 than the second side leg engaging section tab. 715. In one or more embodiments, this design feature allows for the accommodation of the concave curvature of the side leg engaging sections 701,702, as best seen in FIG. 20. By having side leg engaging section tabs 714,715 of different lengths, the heel protector 700 can conform more closely to the natural shape of the leg, providing a more secure and comfortable fit.

One advantage of having side leg engaging section tabs 714,715 of different lengths is improved flexibility and adjustability. The varying lengths allow for a customized fit that can accommodate different leg sizes and shapes. This ensures that the heel protector 700 can be properly secured to the leg, minimizing the risk of slippage or discomfort during use.

Additionally, the different lengths of the side leg engaging section tabs 714,715 contribute to the overall stability and support provided by the heel protector 700. The longer second side leg engaging section tab 715 extends farther from the foot engaging section 704, providing additional reinforcement and preventing excessive movement or shifting of the heel protector 700. This helps to maintain proper alignment and positioning of the heel protector 700, enhancing its effectiveness in providing support and protection to the leg and heel.

Furthermore, the use of side leg engaging section tabs 714,715 of different lengths allows for better weight distribution and pressure relief. The shorter first side leg engaging section tab 714 reduces pressure on specific areas of the leg, distributing the load more evenly. This can help to alleviate discomfort and reduce the risk of pressure-related injuries, such as pressure ulcers or skin breakdown.

Overall, the inclusion of side leg engaging section tabs 714,715 of different lengths in the heel protector 700 offers several advantages. It improves flexibility and adjustability, enhances stability and support, and promotes better weight distribution and pressure relief. These design features contribute to the overall comfort, functionality, and effectiveness of the heel protector, ensuring optimal protection and support for the leg and heel.

As can be best seen in FIGS. 15 and 19, in one or more embodiments, when the second side leg engaging section strap 804 attaches to the second loop fastener panel 805, it covers one of the side leg engaging section tabs 717 that fixedly attaches the foot engaging section 704 to the second side leg engaging section 702 (note that the other side leg engaging section tab 716 that fixedly coupled the foot engaging section 704 to the second side leg engaging section 702 remains exposed). This overlapping arrangement offers several benefits.

Illustrating by example, in one or more embodiments it provides an additional layer of security and stability to the attachment mechanism. The second side leg engaging section strap 804 acts as a reinforcement for the side leg engaging section tab 717, ensuring that the foot engaging section 704 remains securely fastened to the second side leg engaging section 702, even during movement or activity. This helps to prevent any accidental detachment or loosening of the attachment, providing a reliable and secure fit for the wearer.

Next, the overlapping arrangement helps to distribute the tension and stress evenly across the attachment mechanism. By covering one of the side leg engaging section tabs 717, the second side leg engaging section strap 804 helps to distribute the forces exerted on the attachment mechanism, reducing the strain on any single point of connection. This helps to minimize the risk of wear and tear, prolonging the lifespan of the heel protector 700 and ensuring its durability over time.

Additionally, the overlapping arrangement contributes to the overall aesthetics and streamlined design of the heel protector 700. By covering one of the side leg engaging section tabs 717, the second side leg engaging section strap 804 creates a clean and seamless appearance, enhancing the visual appeal of the heel protector 700. This can be particularly beneficial for patients who value both functionality and aesthetics in their medical devices.

Overall, the overlapping arrangement of the side leg engaging section strap 804 covering one of the side leg engaging section tabs offers enhanced security, improved distribution of tension, and a visually pleasing design. These benefits contribute to the overall performance and user experience of the heel protector, ensuring optimal comfort, stability, and longevity.

As can be seen from FIGS. 15-22, in one or more embodiments the heel protector 700, when in the wearable configuration, offers a remarkable aesthetic beauty that is both functional and visually appealing. One notable aspect is the symmetry created by the side leg engaging section straps 712,804 passing diagonally across the loop fastener panels 801,805. This design element not only enhances the overall appearance of the heel protector 700 but also adds a sense of balance and harmony to its visual composition.

The diagonal placement of the side leg engaging section straps 712,804 creates an elegant and dynamic look, drawing the eye along the lines of the heel protector 700. This visual flow adds a touch of sophistication and style to the design, making the heel protector 700 stand out as a fashionable accessory. The symmetry of the side leg engaging section straps 712,804 also contributes to a sense of order and organization, creating a visually pleasing and cohesive aesthetic.

Furthermore, the loop fastener panels 801,805 themselves add to the aesthetic appeal of the heel protector 700. The contrasting colors (where used) and textures of the loop fastener material against the smooth surface of the side leg engaging sections 701,702 create a visually interesting and pleasing contrast. This combination of materials adds depth and dimension to the design, making the heel protector 700 visually engaging from every angle.

The overall aesthetic beauty of the heel protector 700 in the wearable configuration is a testament to the careful attention to detail and design considerations. The symmetry of the side leg engaging section straps 712,804 and the visual interplay between the loop fastener panels 801,805 and the side leg engaging sections 701,703 create a visually striking and harmonious composition. This aesthetic appeal adds an extra layer of enjoyment and satisfaction for the wearer, making the heel protector 700 not only functional but also a stylish accessory.

In summary, FIGS. 15-22 provide a clear visual representation of the different parts and their configuration in the heel protector. It helps understand how the foot engaging section can be selectively attached and detached, providing flexibility and convenience in its use. Worthy of note, heel protectors configured in accordance with embodiments of the disclosure provide several additional advantages:

Versatility: The heel protector 700 can be used in various settings and applications, including medical rehabilitation, sports, and everyday wear. Its adaptable design allows it to provide support and protection in different scenarios, catering to a wide range of user needs.

Ease of Use: The heel protector 700 is designed to be user-friendly and easy to put on and take off. Its intuitive attachment mechanism and adjustable straps ensure a hassle-free experience for both patients and healthcare providers.

Hygiene and Maintenance: The heel protector 700 is constructed with materials that are easy to clean and maintain, promoting good hygiene practices. This is particularly important in medical settings where infection control is crucial.

Durability: Heel protectors configured in accordance with embodiments of the disclosure are built to withstand regular use and provide long-lasting performance. The robust construction and high-quality materials ensure that it can withstand the rigors of daily wear and maintain its effectiveness over time.

Customizability: Heel protectors configured in accordance with embodiments of the disclosure can be customized to fit individual patient needs. They can be adjusted to accommodate different leg sizes and shapes, ensuring a personalized and comfortable fit for each wearer.

Patient Comfort: Heel protectors configured in accordance with embodiments of the disclosure Are designed with patient comfort in mind. The cushioned and ergonomic design helps to alleviate pressure points and provide a comfortable wearing experience, even during extended periods of use.

Enhanced Mobility: The heel protector 700 allows for freedom of movement, enabling patients to maintain their mobility while still receiving the necessary support and protection for their heel and leg.

These additional advantages further highlight the versatility, user-friendliness, durability, and comfort provided by the heel protector 700 of FIGS. 15-22, making it a valuable solution for various applications and user needs. Other advantages will be obvious to those of ordinary skill in the art having the benefit of this disclosure.

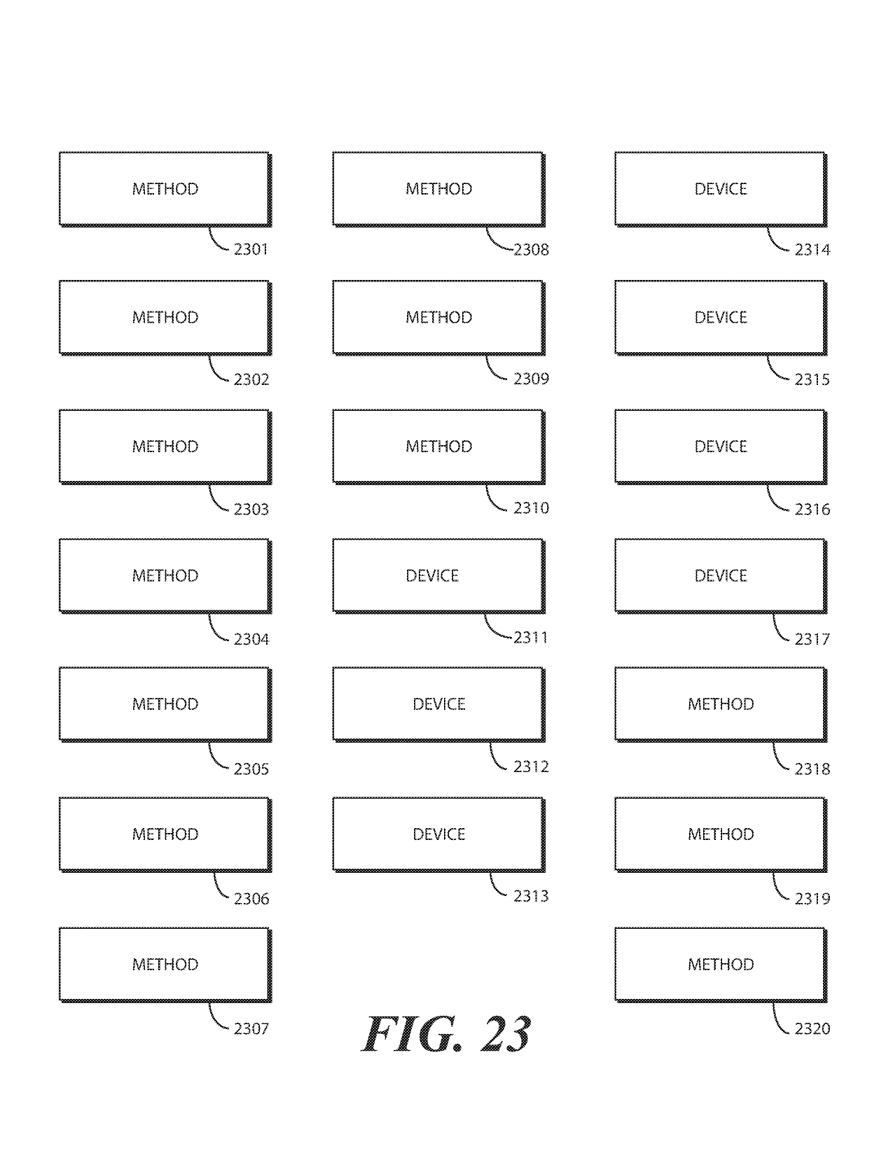

Turning now to FIG. 23, illustrated therein are various embodiments of the disclosure. The embodiments of FIG. 23 are shown as labeled boxes in FIG. 23 due to the fact that the individual components of these embodiments have been illustrated in detail in FIGS. 1-22, which precede FIG. 3. Accordingly, since these items have previously been illustrated and described, their repeated illustration is no longer essential for a proper understanding of these embodiments. Thus, the embodiments are shown as labeled boxes.

At 2301, a heel protector, comprises a leg engaging section. At 2301, the leg engaging section comprises a first side leg engaging section, a second side leg engaging section, and a third side leg engaging section coupled between the first side leg engaging section and the second side leg engaging section. At 2301, a foot engaging section is coupled to one of the first side leg engaging section or the second side leg engaging section and selectively couplable to another of the first side leg engaging section or the second side leg engaging section such that the foot engaging section, the first side leg engaging section, and the second side leg engaging section define a leg insertion aperture when the foot engaging section is selectively coupled to the another of the first side leg engaging section or the second side leg engaging section, and the first side leg engaging section, the second side leg engaging section, the third side leg engaging section, and the foot engaging section can lie in a substantially planar configuration when the foot engaging section is selectively decoupled from the another of the first side leg engaging section or the second side leg engaging section.

At 2302, the third side leg engaging section of 2301 defines a heel receiver. At 2303, the heel receiver of 2302 is defined by a peninsular aperture centrally situated at an edge of the third side leg engaging section.

At 2304, the heel protector of 2301 further comprises a side leg engaging section strap coupled to, and extending distally from, the foot engaging section. At 2304, the side leg engaging section strap is selectively couplable to the one of the first side leg engaging section or the second side leg engaging section.

At 2305, the side leg engaging section of 2304 consists of a single side leg engaging section strap. At 2306, the single side leg engaging section strap of 2305 extends distally away from the foot engaging section at an angle.

At 2307, the leg engaging section of 2306, the first side leg engaging section, the second side leg engaging section, and the third side leg engaging section comprise a compressible cushion layer. At 2308, the compressible cushion layer of 2307 comprises one of an organic batting, an inorganic batting, or combinations thereof.

At 2309, the heel protector of 2305 further comprises a first loop fastener panel coupled to an exterior surface the first side leg engaging section and a second loop fastener panel coupled to an exterior surface of the second side leg engaging section. At 2310, the heel protector of 2304 further comprises at least one side leg engaging section tab coupled to the foot engaging section that is shorter than the side leg engaging section strap and is selectively attachable to the one of the first side leg engaging section or the second side leg engaging section.

At 2311, the heel protector of 2310 further comprises a hook fastener coupled to the at least one side engaging section tab. At 2312, the at least one side leg engaging section tab of 2311 comprises a first side leg engaging section tab and a second side leg engaging section tab.

At 2313, the first side leg engaging section tab of 2312 extends from a first corner section of the foot engaging section and the second side leg engaging section tab extends from a second corner section of the foot engaging section. At 2314, the first side leg engaging section tab of 2313 and the second side leg engaging section tab extend distally different distances from the foot engaging section.

At 2315, at least one of the first side leg engaging section tab of 2314 and the second side leg engaging section tab has at least one rounded corner. At 2316, the foot engaging section of 2301 is coupled to the one of the first side leg engaging section or the second side leg engaging section by a first side leg engaging section tab and a second side leg engaging section tab with the one of the first side leg engaging section or the second side leg engaging section, the foot engaging section, the first side leg engaging section tab, and the second side leg engaging section tab defining a rectangular aperture.

At 2317, a heel protector comprises a leg engaging section defined by a right side, a left side, and a rear side situated between the right side and the left side. At 2317, the heel protector comprises a foot engaging section having a first side coupled to one of the right side or the left side and comprising a side engagement extension coupled to, and extending distally from, a second side the foot engaging section that is separated from the first side of the foot engaging section by a width of the foot engaging section. At 2317, the side engagement extension is selectively attachable to an exterior surface of another of the right side or the second side.

At 2318, the heel protector of 2317 further comprises at least one side engagement tab extending distally from the second side of the foot engaging section. At 2318, the at least one side engagement tab extends distally by a shorter distance from the foot engaging section than the side engagement extension. At 2318, the side engagement extension extends distally from the foot engaging section at a different angle than the at least one side engagement tab.

At 2319, the leg engaging section of 2317 comprises a heel receiver defined by a peninsular aperture. At 2320, an upper edge of the leg engaging section of 2317 is convex and upper edges of the right side and the left side are concave.

In the foregoing specification, specific embodiments of the present disclosure have been described. However, one of ordinary skill in the art appreciates that various modifications and changes can be made without departing from the scope of the present disclosure as set forth in the claims below. Thus, while preferred embodiments of the disclosure have been illustrated and described, it is clear that the disclosure is not so limited. Numerous modifications, changes, variations, substitutions, and equivalents will occur to those skilled in the art without departing from the spirit and scope of the present disclosure as defined by the following claims.

For example, in FIGS. 7-22, 32-35, and 40-45, the foot panel starts higher up, thereby exposing more of the heel. Advantageously, this feature facilitates better air circulation and/or ventilation, better visibility of the heel position in the boot to ensure it is correctly offloaded, and better inspection of the skin integrity of the heel to monitor pressure injury.

Accordingly, the specification and figures are to be regarded in an illustrative rather than a restrictive sense, and all such modifications are intended to be included within the scope of present disclosure. The benefits, advantages, solutions to problems, and any element(s) that may cause any benefit, advantage, or solution to occur or become more pronounced are not to be construed as a critical, required, or essential features or elements of any or all the claims.

What is claimed is:

1. A heel protector, comprising:a leg engaging section, comprising:a first side leg engaging section;a second side leg engaging section; anda third side leg engaging section coupled between the first side leg engaging section and the second side leg engaging section; and a foot engaging section coupled to one of the first side leg engaging section or the second side leg engaging section and selectively couplable to another of the first side leg engaging section or the second side leg engaging section such that the foot engaging section, the first side leg engaging section, and the second side leg engaging section define a leg insertion aperture when the foot engaging section is selectively coupled to the another of the first side leg engaging section or the second side leg engaging section and the first side leg engaging section, the second side leg engaging section, the third side leg engaging section, and the foot engaging section can lie in a substantially planar configuration when the foot engaging section is selectively decoupled from the another of the first side leg engaging section or the second side leg engaging section.

2. The heel protector of claim 1, wherein the third side leg engaging section defines a heel receiver through which a heel can be seen when the heel protector is applied to a patient.

3. The heel protector of claim 2, wherein the heel receiver is defined by a
peninsular aperture centrally situated at an edge of the third side leg engaging section.

4. The heel protector of claim 1, further comprising a side leg engaging section strap coupled to, and extending distally from, the foot engaging section, wherein the side leg engaging section strap is selectively couplable to the one of the first side leg engaging section or the second side leg engaging section.

5. The heel protector of claim 4, wherein the side leg engaging section strap consists of a single side leg engaging section strap.

6. The heel protector of claim 5, wherein the single side leg engaging section strap extends distally away from the foot engaging section at an angle.

7. The heel protector of claim 6, wherein the single side leg engaging section strap comprises a base spanning a width of the foot engaging section tapering to a strap extension extending distally away from the base at the angle.

8. The heel protector of claim 7, wherein the base tapers to the strap extension along a convex single side leg engaging strap contour and a concave single side leg engaging strap contour.

9. The heel protector of claim 5, wherein an edge of the foot engaging section and the single side leg engaging section strap define a triangle.

10. The heel protector of claim 4, further comprising at least one side leg engaging section tab coupled to the foot engaging section that is shorter than the side leg engaging section strap and is selectively attachable to the one of the first side leg engaging section or the second side leg engaging section.

11. The heel protector of claim 10, wherein the at least one side leg engaging section tab consists of a single side leg engaging tab spanning a width of the foot
engaging section.

12. The heel protector of claim 11, wherein the at least one side leg engaging section tab comprises a first side leg engaging section tab and a second side leg engaging section tab.

13. The heel protector of claim 12, wherein the first side leg engaging section tab extends from a first corner section of the foot engaging section and the second side leg engaging section tab extends from a second corner section of the foot engaging section.

14. The heel protector of claim 13, wherein the first side leg engaging section tab and the second side leg engaging section tab extend distally different distances from the foot engaging section.

15. The heel protector of claim 14, wherein at least one of the first side leg engaging section tab and the second side leg engaging section tab has at least one rounded corner.

16. The heel protector of claim 1, wherein the foot engaging section is coupled to the one of the first side leg engaging section or the second side leg engaging section by a first side leg engaging section tab and a second side leg engaging section tab, with the one of the first side leg engaging section or the second side leg engaging section, the foot engaging section, the first side leg engaging section tab, and the second side leg engaging section tab defining a rectangular aperture.

17. A heel protector, comprising:a leg engaging section defined by a right side, a left side, and a rear side situated between the right side and the left side; and
a foot engaging section having a first side coupled to one of the right side or the left side wherein:the foot engaging section is selectively attachable to a first side of the legengaging section or a second side of the leg engaging section such that the foot engaging section and the leg engaging section define a leg insertion aperture when the foot engaging section is selectively coupled to the first side of the leg engaging section or the second side of the leg engaging section; andthe foot engaging section can lie in a substantially planar configuration when the foot engaging section is selectively decoupled from the first side of the leg engaging section or the second side of the leg engaging section and comprising a side engagement extension coupled to, and extending distally from, a second side the foot engaging section that is separated from the first side of the foot engaging section by a width of the foot engaging section;
.

18. The heel protector of claim 17, further comprising at least one side engagement tab extending distally from the second side of the foot engaging section.
.

19. (canceled)

20. (canceled)
